SEQUENCE LISTING (67 sequences)

SEQ ID NO: 1 - DNA, 447 bp, Mouse sp.
  misc_feature (1)..(447): Mouse IL-2 (nucleic acid sequence)
gcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaa

SEQ ID NO: 2 - protein, 149 aa, Mouse sp.
  misc_feature (1)..(149): Mouse IL-2 (amino acid sequence)
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 3 - DNA, 447 bp, Artificial Sequence
  Synthetic construct QQ6210 (nucleic acid sequence)
gcacccacttcaagctccacttcaagctctacagcggaagcacaacagcagcagcagcagcagcagcagcagcagcagcacctggagcagctgttgatggacctacaggaactcctgagtaggatggaggatcacaggaacctgagactccccaggatgctcaccttcaaattttacttgcccgagcaggccacagaattggaagatcttcagtgcctagaagatgaacttgaaccactgcggcaagttctggatttgactcaaagcaaaagctttcaattggaagatgctgagaatttcatcagcaatatcagagtaactgttgtaaaactaaagggctctgacaacacatttgagtgccaattcgacgatgagccagcaactgtggtggactttctgaggagatggatagccttctgtcaaagcatcatctcaacaagccctcaa

SEQ ID NO: 4 - protein, 149 aa, Artificial Sequence
  Synthetic construct QQ6210 (amino acid sequence)
APTSSSTSSSTAEAQQQQQQQQQQQQHLEQLLMDLQELLSRMEDHRNLRLPRMLTFKFYLPEQATELEDLQCLEDELEPLRQVLDLTQSKSFQLEDAENFISNIRVTVVKLKGSDNTFECQFDDEPATVVDFLRRWIAFCQSIISTSPQ

SEQ ID NO: 5 - DNA, 447 bp, Artificial Sequence
  Synthetic construct E76A (nucleic acid sequence)
gcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgctcttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaa

SEQ ID NO: 6 - protein, 149 aa, Artificial Sequence
  Synthetic construct E76A (amino acid sequence)
APTSSSTSSSTAEAQQQQQQQQQQQQHLEQLLMDLQELLSRMENYRNLKLPRMLTFKFYLPKQATELKDLQCLEDALGPLRHVLDLTQSKSFQLEDAENFISNIRVTVVKLKGSDNTFECQFDDESATVVDFLRRWIAFCQSIISTSPQ

SEQ ID NO: 7 - DNA, 447 bp, Artificial Sequence
  Synthetic construct E76G (nucleic acid sequence)
gcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatggtcttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaa

SEQ ID NO: 8 - protein, 149 aa, Artificial Sequence
  Synthetic construct E76G (amino acid sequence)
APTSSSTSSSTAEAQQQQQQQQQQQQHLEQLLMDLQELLSRMENYRNLKLPRMLTFKFYLPKQATELKDLQCLEDGLGPLRHVLDLTQSKSFQLEDAENFISNIRVTVVKLKGSDNTFECQFDDESATVVDFLRRWIAFCQSIISTSPQ

SEQ ID NO: 9 - DNA, 816 bp, Artificial Sequence
  Synthetic construct D265A Fc/Flag (nucleic acid sequence)
atgagggtccccgctcagctcctggggctcctgctgctctggctcccaggtgcacgatgtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggccgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaaggtggcggatctgactacaaggacgacgatgacaagtgataa

SEQ ID NO: 10 - protein, 270 aa, Artificial Sequence
  Synthetic construct D265A Fc/Flag (amino acid sequence)
MRVPAQLLGLLLLWLPGARCEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKGGGSDYKDDDDK

SEQ ID NO: 11 - DNA, 1257 bp, Artificial Sequence
  Synthetic construct D265A Fc/wt mIL-2 (nucleic acid sequence)
atgagggtccccgctcagctcctggggctcctgctgctctggctcccaggtgcacgatgtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggccgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaaggagggggctccgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaacaccatcaccaccatcactgataa

SEQ ID NO: 12 - protein, 417 aa, Artificial Sequence
  Synthetic construct D265A Fc/wt mIL-2 (amino acid sequence)
MRVPAQLLGLLLLWLPGARCEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQHHHHHH

SEQ ID NO: 13 - DNA, 1257 bp, Artificial Sequence
  Synthetic construct D265A Fc /QQ6210 (nucleic acid sequence)
atgagggtccccgctcagctcctggggctcctgctgctctggctcccaggtgcacgatgtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggccgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaaggagggggctccgcacccacttcaagctccacttcaagctctacagcggaagcacaacagcagcagcagcagcagcagcagcagcagcagcacctggagcagctgttgatggacctacaggaactcctgagtaggatggaggatcacaggaacctgagactccccaggatgctcaccttcaaattttacttgcccgagcaggccacagaattggaagatcttcagtgcctagaagatgaacttgaaccactgcggcaagttctggatttgactcaaagcaaaagctttcaattggaagatgctgagaatttcatcagcaatatcagagtaactgttgtaaaactaaagggctctgacaacacatttgagtgccaattcgacgatgagccagcaactgtggtggactttctgaggagatggatagccttctgtcaaagcatcatctcaacaagccctcaacaccatcaccaccatcactgataa

SEQ ID NO: 14 - protein, 417 aa, Artificial Sequence
  Synthetic construct D265A Fc /QQ6210 (amino acid sequence)
MRVPAQLLGLLLLWLPGARCEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKGGGSAPTSSSTSSSTAEAQQQQQQQQQQQQHLEQLLMDLQELLSRMEDHRNLRLPRMLTFKFYLPEQATELEDLQCLEDELEPLRQVLDLTQSKSFQLEDAENFISNIRVTVVKLKGSDNTFECQFDDEPATVVDFLRRWIAFCQSIISTSPQHHHHHH

SEQ ID NO: 15 - DNA, 1257 bp, Artificial Sequence
  Synthetic construct D265A Fc / E76A (nucleic acid sequence)
atgagggtccccgctcagctcctggggctcctgctgctctggctcccaggtgcacgatgtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggccgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaaggagggggctccgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgctcttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaacaccatcaccaccatcactgataa

SEQ ID NO: 16 - protein, 417 aa, Artificial Sequence
  Synthetic construct D265A Fc / E76A (amino acid sequence)
MRVPAQLLGLLLLWLPGARCEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKGGGSAPTSSSTSSSTAEAQQQQQQQQQQQQHLEQLLMDLQELLSRMENYRNLKLPRMLTFKFYLPKQATELKDLQCLEDALGPLRHVLDLTQSKSFQLEDAENFISNIRVTVVKLKGSDNTFECQFDDESATVVDFLRRWIAFCQSIISTSPQHHHHHH

SEQ ID NO: 17 - DNA, 1257 bp, Artificial Sequence
  Synthetic construct D265A Fc / E76G (nucleic acid sequence)
atgagggtccccgctcagctcctggggctcctgctgctctggctcccaggtgcacgatgtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggccgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaaggagggggctccgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatggtcttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaacaccatcaccaccatcactgataa

SEQ ID NO: 18 - protein, 417 aa, Artificial Sequence
  Synthetic construct D265A Fc / E76G (amino acid sequence)
MRVPAQLLGLLLLWLPGARCEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKGGGSAPTSSSTSSSTAEAQQQQQQQQQQQQHLEQLLMDLQELLSRMENYRNLKLPRMLTFKFYLPKQATELKDLQCLEDGLGPLRHVLDLTQSKSFQLEDAENFISNIRVTVVKLKGSDNTFECQFDDESATVVDFLRRWIAFCQSIISTSPQHHHHHH

SEQ ID NO: 19 - DNA, 447 bp, Artificial Sequence
  Synthetic construct mIL-2 QQ 6.2-4 (nucleic acid sequence)
gcacccacctcaagctccacttcaagctctacagcggaagcacaacagcagcagcagcagcagcagcagcagcagcagcacctggagcagctgttgatggacctacaggagctcctgagcaggatggaggattccaggaacctgagactccccaggatgctcaccttcaaattttacttgcccaagcaggccacagaattggaagatcttcagtgcctagaagatgaacttgaacctctgcggcaagttctggatttgactcaaagcaaaagctttcaattggaagatgctgagaatttcatcagcaatatcagagtaactgttgtaaaactaaagggctctgacaacacatttgagtgccaattcgatgatgagccagcaactgtggtgggctttctgaggagatggatagccttctgtcaaagcatcatctcaacgagccctcaa

SEQ ID NO: 20 - protein, 149 aa, Artificial Sequence
  Synthetic construct mIL-2 QQ 6.2-4 (amino acid sequence)
APTSSSTSSSTAEAQQQQQQQQQQQQHLEQLLMDLQELLSRMEDSRNLRLPRMLTFKFYLPKQATELEDLQCLEDELEPLRQVLDLTQSKSFQLEDAENFISNIRVTVVKLKGSDNTFECQFDDEPATVVGFLRRWIAFCQSIISTSPQ

SEQ ID NO: 21 - DNA, 435 bp, Artificial Sequence
  Synthetic construct mIL-2 QQ 6.2-8 (nucleic acid sequence)
gcacccacctcaagctccacttcaagctctacagcggaagcacaacagcagcagcagcagcagcagcacctggagcagctgttgatggacctacaggagctcctgagtaggatggaggatcacaggaacctgagactccccaggatgctcaccttcaaattttacttgcccaagcaggccacagaattggaagatcttcagtgcctagaagatgaacttgaacctctgcggcaagttctggatttgactcaaagcaaaagctttcaattggaagatgctgagaatttcatcagcaatatcagagtaactgttgtaaaactaaagggctctgacaacacatttgagtgccaattcgatgatgagccagcaactgtggtggactttctgaggagatggatagccttctgtcaaagcatcatctcaacaagccctcga

SEQ ID NO: 22 - protein, 145 aa, Artificial Sequence
  Synthetic construct mIL-2 QQ 6.2-8 (amino acid sequence)
APTSSSTSSSTAEAQQQQQQQQHLEQLLMDLQELLSRMEDHRNLRLPRMLTFKFYLPKQATELEDLQCLEDELEPLRQVLDLTQSKSFQLEDAENFISNIRVTVVKLKGSDNTFECQFDDEPATVVDFLRRWIAFCQSIISTSPR

SEQ ID NO: 23 - DNA, 447 bp, Artificial Sequence
  Synthetic construct mIL-2 QQ 6.2-10 (nucleic acid sequence)
gcacccacctcaagctccacttcaagctctacagcggaagcacaacagcagcagcagcagcagcagcagcagcagcagcacctggagcagctgttgatggacctacaggaactcctgagtaggatggaggatcacaggaacctgagactccccaggatgctcaccttcaaattttacttgcccgagcaggccacagaattggaagatcttcagtgcctagaagatgaacttgaaccactgcggcaagttctggatttgactcaaagcaaaagctttcaattggaagatgctgagaatttcatcagcaatatcagagtaactgttgtaaaactaaagggctctgacaacacatttgagtgccaattcgacgatgagccagcaactgtggtggactttctgaggagatggatagccttctgtcaaagcatcatctcaacaagccctcag

SEQ ID NO: 24 - protein, 149 aa, Artificial Sequence; identical to SEQ ID NO: 4
  Synthetic construct mIL-2 QQ 6.2-10 (amino acid sequence)

SEQ ID NO: 25 - DNA, 438 bp, Artificial Sequence
  Synthetic construct mIL-2 QQ 6.2-11 (nucleic acid sequence)
gcacccacctcaagctccacttcaagctctacagcggaagcacaacagcagcagcagcagcagcagcagcacctggagcagctgttgatggacctacaggagctcctgagcaggatggaggattccaggaacctgagactccccagaatgctcaccttcaaattttacttgcccgagcaggccacagaattgaaagatctccagtgcctagaagatgaacttgaacctctgcggcaagttctggatttgactcaaagcaaaagctttcaattggaagatgctgagaatttcatcagcaatatcagagtaactgttgtaaaactaaagggctctgacaacacatttgagtgccaattcgacgatgagccagcaactgtggtggactttctgaggagatggatagccttctgtcaaagcatcatctcaacaagccctcag

SEQ ID NO: 26 - protein, 146 aa, Artificial Sequence
  Synthetic construct mIL-2 QQ 6.2-11 (amino acid sequence)
APTSSSTSSSTAEAQQQQQQQQQHLEQLLMDLQELLSRMEDSRNLRLPRMLTFKFYLPEQATELKDLQCLEDELEPLRQVLDLTQSKSFQLEDAENFISNIRVTVVKLKGSDNTFECQFDDEPATVVDFLRRWIAFCQSIISTSPQ

SEQ ID NO: 27 - DNA, 447 bp, Artificial Sequence
  Synthetic construct mIL-2 QQ 6.2-13 (nucleic acid sequence)
gcacccacctcaagctccacttcaagctctacagcggaagcacaacagcagcagcagcagcagcagcagcagcagcagcacctggagcagctgttgatggacctacaggagctcctgagtaggatggaggatcacaggaacctgagactccccaggatgctcaccttcaaattttacttgcccgagcaggccacagaattgaaagatctccagtgcctagaagatgaacttgaacctctgcggcaggttctggatttgactcaaagcaaaagctttcaattggaagatgctgagaatttcatcagcaatatcagagtaactgttgtaaaactaaagggctctgacaacacatttgagtgccaattcgatgatgagccagcaactgtggtggactttctgaggagatggatagccttctgtcaaagcatcatctcaacaagccctcag

SEQ ID NO: 28 - protein, 149 aa, Artificial Sequence
  Synthetic construct mIL-2 QQ 6.2-13 (amino acid sequence)
APTSSSTSSSTAEAQQQQQQQQQQQQHLEQLLMDLQELLSRMEDHRNLRLPRMLTFKFYLPEQATELKDLQCLEDELEPLRQVLDLTQSKSFQLEDAENFISNIRVTVVKLKGSDNTFECQFDDEPATVVDFLRRWIAFCQSIISTSPQ

SEQ ID NO: 29 - DNA, 461 bp, Homo sapiens
  misc_feature (1)..(461): Full length human IL-2 (nucleic acid sequence)
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtaatatgctgatgagacagcaaccattgtagaatttctgaacagatggattaccttttgtcaaagcatcatctcaacactgacttga

SEQ ID NO: 30 - protein, 153 aa, Homo sapiens
  misc_feature (1)..(153): Full length human IL-2 (amino acid sequence)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 31 - DNA, 401 bp, Homo sapiens
  misc_feature (1)..(401): Human IL-2 without signal peptide (nucleic acid sequence)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtaatatgctgatgagacagcaaccattgtagaatttctgaacagatggattaccttttgtcaaagcatcatctcaacactgacttga

SEQ ID NO: 32 - protein, 133 aa, Homo sapiens [sequence omitted]

SEQ ID NO: 33 - protein, 330 aa, Homo sapiens [sequence omitted]

SEQ ID NO: 34 - protein, 232 aa, Homo sapiens
  misc_feature (1)..(232): Human IgG1 Fc domain (amino acid sequence)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 609 aa, Homo sapiens [sequence omitted]

SEQ ID NO: 36 - protein, 585 aa, Homo sapiens [sequence omitted]

SEQ ID NO: 37 - DNA, 1755 bp, Homo sapiens
  misc_feature (1)..(1755): Mature HSA (nucleic acid sequence)
gatgctcacaaaagcgaagtcgcacacaggttcaaagatctgggggaggaaaactttaaggctctggtgctgattgcattcgcccagtacctgcagcagtgcccctttgaggaccacgtgaaactggtcaacgaagtgactgagttcgccaagacctgcgtggccgacgaatctgctgagaattgtgataaaagtctgcatactctgtttggggataagctgtgtacagtggccactctgcgagaaacctatggagagatggcagactgctgtgccaaacaggaacccgagcggaacgaatgcttcctgcagcataaggacgataaccccaatctgcctcgcctggtgcgacctgaggtggacgtcatgtgtacagccttccacgataatgaggaaacttttctgaagaaatacctgtacgaaatcgctcggagacatccttacttttatgcaccagagctgctgttctttgccaaacgctacaaggccgctttcaccgagtgctgtcaggcagccgataaagctgcatgcctgctgcctaagctggacgaactgagggatgagggcaaggccagctccgctaaacagcgcctgaagtgtgctagcctgcagaaattcggggagcgagccttcaaggcttgggcagtggcacggctgagtcagagattcccaaaggcagaatttgccgaggtctcaaaactggtgaccgacctgacaaaggtgcacaccgaatgctgtcatggcgacctgctggagtgcgccgacgatcgagctgatctggcaaagtatatttgtgagaaccaggactccatctctagtaagctgaaagaatgctgtgagaaaccactgctggaaaagtctcactgcattgccgaagtggagaacgacgagatgccagctgatctgccctcactggccgctgacttcgtcgaaagcaaagatgtgtgtaagaattacgctgaggcaaaggatgtgttcctgggaatgtttctgtacgagtatgccaggcgccacccagactactccgtggtcctgctgctgaggctggctaaaacatatgaaaccacactggagaagtgctgtgcagccgctgatccccatgaatgctatgccaaagtcttcgacgagtttaagcccctggtggaggaacctcagaacctgatcaaacagaattgtgaactgtttgagcagctgggcgagtacaagttccagaacgccctgctggtgcgctataccaagaaagtcccacaggtgtccacacccactctggtggaggtgagccggaatctgggcaaagtggggagtaaatgctgtaagcaccctgaagccaagaggatgccatgcgctgaggattacctgagtgtggtcctgaatcagctgtgtgtcctgcatgaaaaaacacctgtcagcgaccgggtgacaaagtgctgtactgagtcactggtgaaccgacggccctgctttagcgccctggaagtcgatgagacttatgtgcctaaagagttcaacgctgagaccttcacatttcacgcagacatttgtaccctgagcgaaaaggagagacagatcaagaaacagacagccctggtcgaactggtgaagcataaacccaaggccacaaaagagcagctgaaggctgtcatggacgatttcgcagcctttgtggaaaaatgctgtaaggcagacgataaggagacttgctttgccgaggaaggaaagaaactggtggctgcatcccaggcagctctgggactg

SEQ ID NO: 38 - protein, 369 aa, Artificial Sequence
  Synthetic construct hFc/hIL-2 fusion
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 39 - protein, 369 aa, Artificial Sequence
  Synthetic construct hIL-2/hFc
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 722 aa, Artificial Sequence
  Synthetic construct HSA/hIL-2 fusion
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 41 - protein, 722 aa, Artificial Sequence
  Synthetic construct hIL-2/HSA fusion
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 42 - protein, 149 aa, Artificial Sequence
  Synthetic construct 6.2-6
APTSSSTSSSTAEAQQQQQQQQQQQRHLEQLSMDLQELLSRMEDSRNLRLPRMLTFKFYLPEQATELKDLQCLEDELEPLRQVLDLTQSKSFQLEDAEDFISNIRVTVAKLRGSDNTFECQFDDEPATVVDFLRRWIAFCQSIISTSPQ

SEQ ID NO: 43 - protein, 148 aa, Artificial Sequence
  Synthetic construct 6.2-9
APTSSSTSSSTAEAQQQQQQQQQQRHLEQLSMDLQELLSRMEDSRNLRLPRMLTFKFYLPEQATELKDLQCLEDELEPLRQVLDLTQSKSFQLEDAEDFISNIRVTVAKLKGSDNTFECQFDDEPATVVDFLRRWIAFCQSIISTSPQ

SEQ ID NO: 44 - protein, 149 aa, Artificial Sequence
  Synthetic construct 6.2-11
APTSSSTSSSTAEAQQQQQQQQQQQRHLEQLSMDLQELLSRMEDSRNLRLPRMLTFKFYLPEQATELKDLQCLEDELEPLRQVLDLTQSKSFQLEDAEDFISNIRVTVAKLKGSDNTFECQFDDEPATVVDFLRRWIAFCQSIISTSPQ

SEQ ID NO: 45 - protein, 149 aa, Artificial Sequence
  Synthetic construct 6.2-12
APTSSSTSSSTAEAQQQQQQQQQQQRHLEQLSVDLQELLSRMEDSRNLRLPRMLTFKFYLPEQATELKDLQCLEDELEPLRQVLDLTQSKSFQLEGAENFISNIRVTVAKLKGSDNTFECQFDDEPATVVDFLRRWIAFCQSIISTSPQ

SEQ ID NO: 46 - protein, 144 aa, Artificial Sequence
  Synthetic construct 6.2-2
APTSSSTSSSTAEAQQQQQQQHLEQLSMDLQELLSRMEDHRNLRLPRMLTFKFYLPEQATELKDLQCLEDELEPLRQVLDLTQSKSFQLEGAENFISNIRATVVKLKGSDNTFECQFDDEPATVVDFLRRWIAFCQSIISTSPQ

SEQ ID NO: 47 - protein, 149 aa, Artificial Sequence
  Synthetic construct 6.2-13
APTSSSTSSSTAEAQQQQQQQQQQQRHLEQLLMDLQELLSRMEDHRNLRLPRMLTFKFYLPEQATELKDLQCLEDELEPLRQVLDLTQSKSFQLEGAENFISNIRATVVKLKGSDNTFECQFDDEPATVVDFLRRWIAFCQSIISTSPQ

SEQ ID NO: 48 - protein, 149 aa, Artificial Sequence
  Synthetic construct 6.2-7
APTSSSTSSSTAEAQQQQQQQQQQRRHLEQLLMDLQELLSRMEDHRNLRLPRMLTFKFYLPEQATELKDLQCLEDELEPLRQVLDLTQSKSFQLEGAENFISNIRATVVKLKGSDNTFECQFDDEPATVVDFLRRWIAFCQSIISTSPQ

SEQ ID NO: 49 - protein, 149 aa, Artificial Sequence
  Synthetic construct 6.2-1
APTSSSTSSSTAEAQQQQQQQQQQQRHLEQLLMDLQELLSRMEDHRNLRLPRMLTFKFYLPEQATELKDLQCLEDELEPLRQVLDLTQSKSFQLEGAENFISNIRVTVAKLKGSDSTFECQFDDEPATVVGFLRRWIAFCQSIISTSPQ

SEQ ID NO: 50 - protein, 149 aa, Artificial Sequence
  Synthetic construct 6.2-10
APTSSSTSSSTAEAQQQQQQQQQQQRHLEQLLMDLQELLSRMEDHRNLRLPRMLTFKFYLPEQATELEDLQCLEDELEPLRQVLDLTQSKSFQLEDAEDFISNIRVTVAKLKGSDNTFECQFDDEPATVVDFLRRWIAFCQSIISTSPQ

SEQ ID NO: 51 - protein, 148 aa, Artificial Sequence
  Synthetic construct 6.2-8
APTSSSTSSSTAEAQQQQQQQQQQRHLEQLLMDLQELLSRMEDHRNLRLPRMLTFKFYLPKQATELEDLQCLEDELEPLRQVLDLTQSKSFQLEGAENFISNIRATVVKLKGSDNTFECQFDDEPATVVDFLRRWIAFCQSIISTSPR

SEQ ID NO: 52 - protein, 148 aa, Artificial Sequence
  Synthetic construct 6.2-15
APTSSSTSSSTAEAQQQQQQQQQQRHLEQLLMDLQELLSRMEDHRNLRLPRMLTFKFYLPKQATELEDLQCLEDELEPLRQVLDLTQSKSFQLEGAENFISNIRVTVAKLKGSDSTFECQFDDEPATVVGFLRRWIAFCQSIISTSPQ

SEQ ID NO: 53 - protein, 149 aa, Artificial Sequence
  Synthetic construct 6.2-16
APTSSSTSSSTAEAQQQQQQQQQQQRHLEQLSMDLQELLSRMEDSRNLRLPRMLTFKFYLPKQATELEDLQCLEDELEPLRQVLDLTQSKSFQLEGAENFISNIRATVVKLKGSDSTFECQFDDEPATVVGFLRRWIAFCQSIISTSPQ

SEQ ID NO: 54 - protein, 149 aa, Artificial Sequence; identical to SEQ ID NO: 53
  Synthetic construct 6.2-3

SEQ ID NO: 55 - protein, 145 aa, Artificial Sequence
  Synthetic construct 6.2-5
APTSSSTSSSTAEAQQQQQQQRHLEQLSMDLQELLSRMEDSRNLRLPRMLTFKFYLPKQATELEDLQCLEDELEPLRQVLDLTQSKSFQLEGAENFISDIRVAVAKLKGSDNTFECQFDDEPATVVDFLRRWIAFCQSIISTSPQ

SEQ ID NO: 56 - protein, 149 aa, Artificial Sequence
  Synthetic construct 6.2-14
APTSSSTSSSTAEAQQQQQQQQQQQRHLEQLSMDLQELLSRMEDSRNLRLPRMLTFKFYLPKQATELEDLQCLEDELEPLRQVLDLTQSKSFQLEGAENFISDIRVAVVKLKGSDNTFECQFDDEPATVVGFLRRWIAFCQSIISTSPQ

SEQ ID NO: 57 - protein, 149 aa, Artificial Sequence
  Synthetic construct 6.2-4
APTSSSTSSSTAEAQQQQQQQQQQQQHLEQLSMDLQELLSRMEDSRNLRLPRMLTFKFYLPKQATELEDLQCLEDELEPLRQVLDLTQSKSFQLEDAEDFISNIRVTVAKLKGSDNTFECQFDDEPATVVGFLRRWIAFCQSIISTSPQ

SEQ ID NO: 58 - protein, 149 aa, Artificial Sequence; identical to SEQ ID NO: 20
  Synthetic construct QQ-6.2-4

SEQ ID NO: 59 - protein, 145 aa, Artificial Sequence; identical to SEQ ID NO: 22
  Synthetic construct QQ-6.2-8

SEQ ID NO: 60 - protein, 149 aa, Artificial Sequence; identical to SEQ ID NO: 4
  Synthetic construct QQ-6.2-10

SEQ ID NO: 61 - protein, 146 aa, Artificial Sequence; identical to SEQ ID NO: 26
  Synthetic construct QQ-6.2-11

SEQ ID NO: 62 - protein, 149 aa, Artificial Sequence; identical to SEQ ID NO: 28
  Synthetic construct QQ-6.2-13

SEQ ID NO: 63 - protein, 51 aa, Artificial Sequence
  Synthetic peptide
SGGGGSXXXXXXXXXXXXXXXXXXXXXXXXXXXXXXXXXXXXXXXXXXXXX

SEQ ID NO: 64 - protein, 16 aa, Artificial Sequence
  Synthetic peptide
SGGGGSGGGGSGGGGS

SEQ ID NO: 65 - protein, 21 aa, Artificial Sequence
  Synthetic peptide
SGGGGSGGGGSGGGGSGGGGS

SEQ ID NO: 66 - protein, 30 aa, Artificial Sequence
  Synthetic peptide
GGGGSXXXXXXXXXXXXXXXXXXXXXXXXX

SEQ ID NO: 67 - protein, 24 aa, Artificial Sequence
  Synthetic peptide
GGGSXXXXXXXXXXXXXXXXXXXX

(19) United States
(12) Patent Application Publication
(10) Pub. No.: US 2015/0017120 A1
(43) Pub. Date: Jan. 15, 2015

(54) SYNERGISTIC TUMOR TREATMENT WITH EXTENDED-PK IL-2 AND ADOPTIVE CELL THERAPY

(71) Applicant: MASSACHUSETTS INSTITUTE OF TECHNOLOGY, Cambridge, MA (US)

(72) Inventors: Karl Dane WITTRUP, Chestnut Hill, MA (US); Cary Francis OPEL, Somerville, MA (US); Eric Franklin ZHU, Cambridge, MA (US); Shuning GAI, Eugene, OR (US)

(21) Appl. No.: 14/304,438

(22) Filed: Jun. 13, 2014

Related U.S. Application Data
(60) Provisional application No. 61/834,862, filed on Jun. 13, 2013.

(51) Int. Cl. A61K 38/20; C07K 14/55; C07K 16/30; A61K 47/48; A61K 35/14; A61K 39/395

(52) U.S. Cl. CPC A61K 38/2013; A61K 35/17; A61K 39/39558; C07K 16/30; A61K 47/48215; C07K 14/55; A61K 2039/505
USPC 424/85.2

(57) ABSTRACT

The present invention provides a method of enhancing adoptive cell therapy (ACT) by administering an extended-pharmacokinetic (PK) interleukin (IL)-2 to a cancer subject receiving ACT, optionally in combination with a therapeutic antibody. Methods of treating cancer and promoting tumor regression are also provided.

### CROSS-REFERENCE TO RELATED APPLICATIONS

This application claims priority to and the benefit of U.S. Provisional Patent Application No. 61/834,862, filed Jun. 13, 2013, the entire contents of which is herein incorporated by reference.

The patent or application file contains at least one drawing executed in color. Copies of this patent or patent application publication with color drawing(s) will be provided by the Office upon request and payment of the necessary fee.

### BACKGROUND

Adoptive cell therapy (ACT) is a treatment method in which cells are removed from a donor, cultured and/or manipulated in vitro, and then administered to a patient for the treatment of a disease. In many instances, the cells administered to a patient are autologous cells, meaning that the patient acts as his or her own donor.

A variety of cell types have been used in ACT in an attempt to treat several classes of disorders. For the treatment of cancer, ACT generally involves the transfer of lymphocytes. There are currently several medical research centers testing a variety of T cell-based ACT regimens in cancer patients, but the results of ACT monotherapy have been marginal. This is due in part to the difficulty in promoting the long-term proliferation and survival of the transferred cells. Accordingly, novel approaches are needed to improve the outcome of ACT in cancer patients.

### SUMMARY

To overcome the obstacle of proliferation and persistence of transferred cells, several supporting therapies have been tested in conjunction with ACT, mainly in a preclinical setting. These include patient preconditioning, cancer vaccines, cytokine therapy, and antibodies. Interleukin (IL)-2 is one such supporting therapy that has been administered alongside ACT. IL-2 stimulates T cell proliferation and survival; however, this cytokine has a poor pharmacokinetic profile and severely negative side effects.

The present invention is based, in part, on the discovery that administration of IL-2 attached to a pharmacokinetic modifying group (hereafter referred to as “extended-pharmacokinetic (PK) IL-2”) significantly improves the efficacy of ACT. In particular, administration of extended-PK IL-2 to cancer subjects in combination with ACT increases the persistence of transferred cells, reduces tumor burden, and prolongs survival relative to ACT monotherapy. This effect can be further enhanced by administration of a therapeutic agent. For example, a combination therapy for cancer is provided that involves the administration of extended-PK IL-2 and a therapeutic antibody in conjunction with ACT.

Accordingly, in one aspect, the invention provides a method of prolonging persistence of transferred cells in a cancer subject receiving adoptive cell therapy (ACT), by administering an extended-pharmacokinetic (PK) interleukin (IL)-2 to a cancer subject receiving ACT, in an amount effective to prolong the persistence of transferred cells in the subject.

In another aspect, the invention provides a method of stimulating proliferation of transferred cells in a cancer subject receiving ACT, by administering an extended-pharmacokinetic (PK) interleukin (IL)-2 to a cancer subject receiving ACT, in an amount effective to stimulate proliferation of transferred cells in the subject.

In one aspect, the invention provides a method of stimulating a T cell-mediated immune response to a target cell population in a cancer subject receiving ACT, by administering an extended-pharmacokinetic (PK) interleukin (IL)-2 to a cancer subject receiving ACT, in an amount effective to stimulate a T cell-mediated immune response to a target cell population.

In another aspect, the invention provides a method of treating cancer in a subject, comprising administering to the subject an adoptive cell therapy (ACT), and an extended-pharmacokinetic (PK) interleukin (IL)-2, in an amount effective to treat cancer.

In another aspect, the invention provides a method of promoting tumor regression in a subject, comprising administering to the subject an adoptive cell therapy (ACT), and an extended-pharmacokinetic (PK) interleukin (IL)-2, in an amount effective to promote regression of the tumor in the subject.

In any of the foregoing aspects, the methods may further comprise administering a therapeutic antibody or antibody fragment to the subject. In one embodiment, the therapeutic antibody or antibody fragment specifically recognizes a tumor antigen.

In one embodiment of the foregoing aspects, the ACT comprises administration of autologous cells, e.g., autologous T cells. In one embodiment, the autologous cells are tumor infiltrating lymphocytes (TIL) that have been expanded in vitro. In another embodiment, the autologous cells are CD8+ and/or CD4+ T cells that have been expanded in vitro in the presence of an antigen. In one embodiment, the autologous cells are genetically engineered T cells. In certain embodiments, the genetically engineered T cells have been engineered to express a T cell receptor (TCR) that specifically recognizes a tumor antigen. In another embodiment, the genetically engineered T cells have been engineered to express a chimeric antigen receptor (CAR). In one embodiment, the CAR contains an antigen binding domain, a costimulatory domain, and a CD3 zeta signaling domain. In one embodiment, the antigen binding domain is an antibody or antibody fragment that specifically binds to a tumor antigen. The antibody fragment may be, for example, a Fab or an scFv. In one embodiment, the costimulatory domain contains the intracellular domain of a costimulatory molecule such as 4-1BB, CD27, CD28, OX40, CD30, CD40, PD-1, ICOS, lymphocyte function-associated antigen-1 (LFA-1), CD2, CD7, LIGHT, NKG2C, B7-H3, a CD83 ligand, or combinations thereof. In one embodiment, the costimulatory domain comprises the intracellular domain of 4-1BB.

In one embodiment of the foregoing aspects, the tumor antigen can be an antigen associated with a cancer such as a hematological tumor, a carcinoma, a blastoma, or a sarcoma, e.g., a melanoma or acute myelogenous leukemia. In one embodiment, the tumor antigen is selected from the group consisting of MART-1, gp100, p53, NY-ESO-1, TRP-2, tyrosinase, CD19, CD20, mesothelin, and TRP-1.

In certain of the foregoing aspects, a method of prolonging persistence of transferred cells in a cancer subject receiving adoptive cell therapy (ACT) is provided. In one embodiment, the transferred cells persist for 20% longer, 30% longer, 40% longer, 50% longer, or more in the subject relative to a subject receiving ACT monotherapy. In one embodiment, the invention provides a method of prolonging persistence of transferred cells in a cancer subject receiving adoptive cell therapy (ACT), by administering an extended-pharmacokinetic (PK) interleukin (IL)-2 to a cancer subject receiving ACT, wherein ACT comprises administration of autologous T cells genetically engineered to express a chimeric antigen receptor (CAR), and administering a therapeutic antibody to the subject, wherein the therapeutic antibody and the CAR recognize the same tumor antigen, such that the persistence of transferred cells in the subject is prolonged.

In one embodiment of the foregoing aspects, the extended-PK IL-2 comprises a fusion protein. In another embodiment, the fusion protein comprises an IL-2 moiety and a moiety selected from the group consisting of an immunoglobulin fragment, human serum albumin, and Fn3. In another embodiment, the extended-PK IL-2 comprises an IL-2 moiety conjugated to a non-protein polymer, e.g., polyethylene glycol. In one embodiment, the fusion protein comprises an IL-2 moiety and an Fc domain. In one embodiment, the Fc domain is mutated to reduce binding to Fcγ receptors, complement proteins, or both. In another embodiment, the fusion protein comprises a monomer of one IL-2 moiety linked to an Fc domain as a heterodimer. In one embodiment, the fusion protein comprises a dimer of two IL-2 moieties linked to an Fc domain as a heterodimer. In one embodiment of the foregoing aspects, the IL-2 is mutated such that it has higher affinity for the IL-2R alpha receptor compared to unmodified IL-2.

### BRIEF DESCRIPTION OF THE DRAWINGS

These and other features, aspects, and advantages of the present invention will become better understood with regard to the following description, and accompanying drawings, where:

FIG. 1 depicts the sequences of high affinity CD25-binding mouse IL-2 mutants generated by error prone PCR and yeast surface display. mIL-2 depicts the sequence of murine IL-2. The locations of mutations in the IL-2 mutants are shown. The mutants with names preceded by “QQ” are those in which putative IL-2Rβ-binding mutations were reverted back to wild-type residues by site directed mutagenesis.

FIG. 2 is a series of graphs depicting the affinity of the indicated IL-2 mutants for soluble murine CD25. The equilibrium dissociation constant was determined as described in Chao et al. (Nat Protocols 2006; 1(2):755-768). Diamonds indicate wild-type murine IL-2; squares indicate IL-2 6.2-10; triangles indicate IL-2 mutants in which putative IL-2Rβ-binding mutations were reverted back to wild-type residues.

FIG. 3 is a three dimensional model of murine IL-2 bound to murine CD25 generated using SWISS-MODEL (Schwede et al., Nucleic Acids Research 2003; 31:3381-5). Residues E76, H82, and Q121 are in close contact with CD25.

FIG. 4 is a series of flow cytometry histograms showing the display of E76A IL-2 on the surface of yeast (as determined by anti-HA and anti-c-myc staining), its lack of detectable binding to soluble murine CD25 at 50 nM, and its proper folding (as detected by anti-IL-2 antibodies S4B6, JES6-1A12, and JESA-5H4 before and after thermal denaturation).

FIG. 5 is a schematic of D265AFc/IL-2 (hereafter referred to as “Fc/IL-2”). IL-2 is monovalent and has a K_{D} of about 50 nM for mouse CD25. The beta half-life of Fc/IL-2 is about 15 hours.

FIG. 6 is a series of graphs depicting the viability of CTLL-2 cells stimulated with the indicated Fc/IL-2 and mutants. CTLL-2 cells were stimulated with Fc/IL-2, Fc/QQ6210, Fc/E76A, or Fc/E76G for 30 minutes, then resuspended in cytokine-free medium. At indicated times after cytokine withdrawal, culture aliquots were used to measure culture viability as determined by cellular ATP content, which was assayed through stimulation of ATP-dependent luciferase activity using the CellTiter-Glo Luminescent Viability Assay (Promega).

FIG. 7 is a photograph of spleens isolated from C57BL/6 mice (n=3/group) injected intravenously with PBS or 25 μg Fc/IL-2, Fc/QQ6210, or Fc/E76G. Spleens were isolated 4 days after treatment. Two representative spleens per group are shown.

FIG. 8 is a series of graphs depicting various lymphocyte populations in spleens isolated from mice treated under the conditions described in FIG. 7. Populations of cell types are as indicated. CD3+CD8+ depicts CD8+ T cells, and CD3-NK1.1+ depicts natural killer (NK) cells. Error bars represent standard deviation for measurements of three samples.

FIG. 9 is a graph depicting total weight change (grams), which is used as a proxy for toxicity, in C57BL/6 mice injected with PBS, Fc/IL-2, Fc/QQ6210, or Fc/E76G as described in FIG. 7.

FIG. 10 is a graph depicting total lung wet weight (grams), which is used as an indicator of pulmonary edema and vascular leak syndrome. C57BL/6 mice injected with PBS, Fc/IL-2, Fc/QQ6210, or Fc/E76G as described in FIG. 7.

FIG. 11 depicts the pmel-1 mouse model representative of ACT.

FIG. 12 describes the treatments administered to five groups of C57BL/6 host mice in a study conducted to examine the effect of Fc-IL-2 on ACT.

FIG. 13 presents a timeline detailing the treatment regimen for mice participating in the ACT combination therapy study.

FIG. 14 is a graph depicting tumor area measurements over the course of treatment for each mouse in the ACT combination therapy study.

FIG. 15 presents the mean tumor area measurements and confidence intervals for the data depicted in FIG. 14.

FIG. 16 is a series of graphs depicting the Kaplan-Meier Survival Curves for each treatment group in the ACT combination therapy study.

FIG. 17 depicts bioluminescence imaging of mice following ACT transplantation of donor cells from the pmel-1-luc mouse strain.

FIG. 18 is a graph quantifying the bioluminescence data from FIG. 17.

FIG. 19 depicts bioluminescence imaging of mice following ACT transplantation of donor cells from pmel-1-luc mouse strain after 128 days. Shown are the four surviving ACT combination (ACT+Fc/IL-2+TA99) treated mice and the single surviving combination (Fc/IL-2+TA99) treated mouse (as a negative control).

FIG. 20 is a graph showing the persistence of the transferred cells in FIG. 19 in response to treatment with hgp-100 peptide and cytokine (IFN-γ or TNFα). “Combo” refers to the combination of Fc/IL-2 and TA99. “ACT Combo” refers to the combination of pmel-1 T cells, Fc/IL-2, and TA99.

### DETAILED DESCRIPTION

### Definitions

Terms used in the claims and specification are defined as set forth below unless otherwise specified. In the case of direct conflict with a term used in a parent provisional patent application, the term used in the instant specification shall control.

“Adoptive cell transfer or therapy (ACT)” is a treatment method in which cells are removed from a donor, cultured and/or manipulated in vitro, and administered to a patient for the treatment of a disease. In some embodiments, the transferred cells are autologous cells, meaning that the patient acts as his or her own donor. In some embodiments, the transferred cells are lymphocytes, e.g., T cells. In some embodiments, the transferred cells are genetically engineered prior to administration to a patient. For example, the transferred cells can be engineered to express a T cell receptor (TCR) having specificity for an antigen of interest. In one embodiment, transferred cells are engineered to express a chimeric antigen receptor (CAR). In certain embodiments, transferred cells are engineered (e.g., by transfection or conjugation) to express a molecule that enhances the anti-tumor activity of the cells, such as a cytokine (IL-2, IL-12), an anti-apoptotic molecule (BCL-2, BCL-X), or a chemokine (CXCR2, CCR4, CCR2B). In certain embodiments, T cells are engineered to express both a CAR and a molecule that enhances anti-tumor activity or persistence of cells.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mol. Cell. Probes 8:91-98, 1994). For arginine and leucine, modifications at the second base can also be conservative. The term nucleic acid is used interchangeably with gene, cDNA, and mRNA encoded by a gene. Polynucleotides of the present invention can be composed of any polyribonucleotide or polydeoxyribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include PEG, human serum albumin (HSA) binders (as disclosed in U.S. Publication Nos. 2005/0287153 and 2007/0003549, PCT Publication Nos. WO 2009/083804 and WO 2009/133208, and SABA molecules as described in US2012/094909), human serum albumin, Fc or Fc fragments and variants thereof, transferrin or variants thereof, and sugars (e.g., sialic acid). Other exemplary extended-PK groups are disclosed in Kontermann et al., Current Opinion in Biotechnology 2011; 22:868-876, which is herein incorporated by reference in its entirety. As used herein, an “extended-PK IL-2” refers to an IL-2 moiety in combination with an extended-PK group. In one embodiment, the extended-PK IL-2 is a fusion protein in which an IL-2 moiety is linked or fused to an extended-PK group. An exemplary fusion protein is a Fc/IL-2 fusion in which one or more IL-2 moieties are linked to an immunoglobulin Fc domain (e.g., an IgG1 Fc domain). Another exemplary fusion protein is a human Fc/human IL-2 or human IL-2/human Fc fusion having the amino acid sequence set forth in SEQ ID NO: 38 and 39, respectively, wherein the human IL-2 and human Fc are optionally fused by a linker. Another exemplary fusion protein is a HSA/human IL-2 fusion or a human IL-2/HSA fusion having the amino acid sequence set forth in SEQ ID NO: 40 and 41, respectively, wherein the human IL-2 and HSA are optionally fused by a linker. In certain embodiments, the IL-2 portion of the fusion protein is a mutant IL-2 protein or fragment thereof, as described infra.

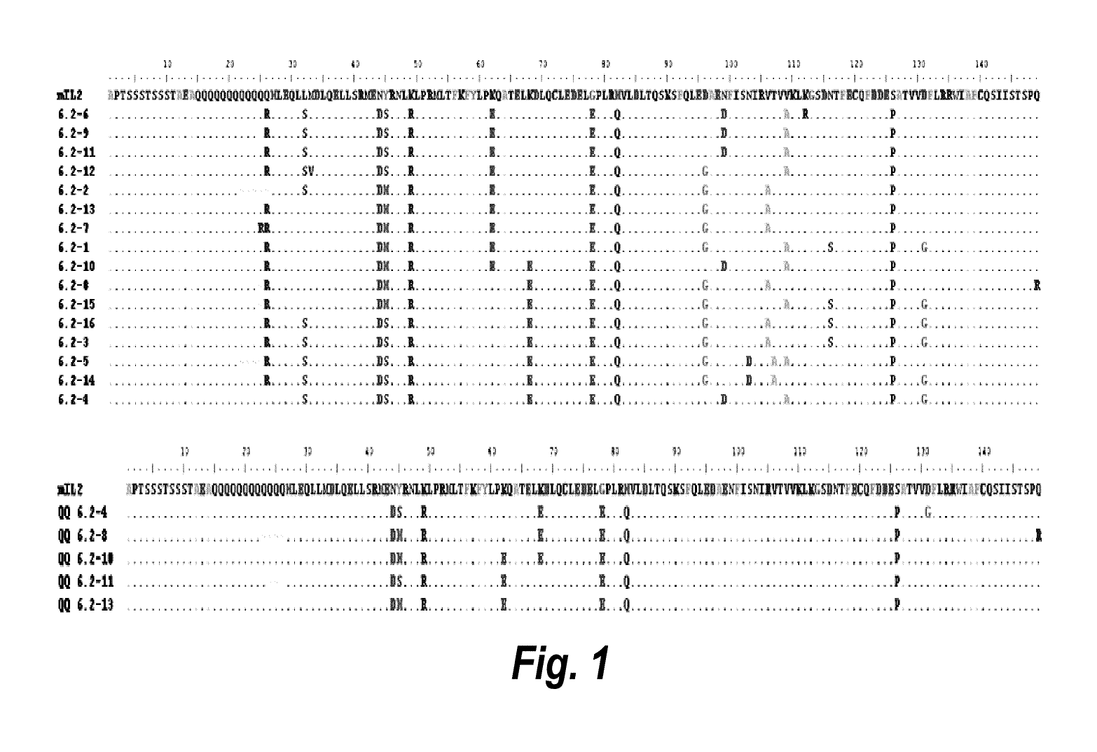

The term “extended-PK IL-2” is also intended to encompass IL-2 mutants with mutations in one or more amino acid residues that enhances the affinity of IL-2 for one or more of its receptors, for example, CD25. In one embodiment, the IL-2 moiety of extended-PK IL-2 is wild-type IL-2. In another embodiment, the IL-2 moiety is a mutant IL-2 which exhibits greater affinity for CD25 than wild-type IL2, such as one of the IL-2 mutants depicted in FIG. 1. When a particular type of extended-PK group is indicated, such as PEG-IL-2, it should be understood that this encompasses both PEG conjugated to a wild-type IL-2 moiety or a PEG conjugated to a mutant IL-2 moiety.

In certain aspects, the extended-PK IL-2 of the invention can employ one or more “linker domains,” such as polypeptide linkers. As used herein, the term “linker domain” refers to a sequence which connects two or more domains (e.g., the PK moiety and IL-2) in a linear sequence. As used herein, the term “polypeptide linker” refers to a peptide or polypeptide sequence (e.g., a synthetic peptide or polypeptide sequence) which connects two or more domains in a linear amino acid sequence of a polypeptide chain. For example, polypeptide linkers may be used to connect an IL-2 moiety to an Fc domain. Preferably, such polypeptide linkers can provide flexibility to the polypeptide molecule. In certain embodiments the polypeptide linker is used to connect (e.g., genetically fuse) one or more Fc domains and/or IL-2.

As used herein, the terms “linked,” “fused”, or “fusion”, are used interchangeably. These terms refer to the joining together of two more elements or components or domains, by whatever means including chemical conjugation or recombinant means. Methods of chemical conjugation (e.g., using heterobifunctional crosslinking agents) are known in the art.

As used herein, the term “Fc region” shall be defined as the portion of a native immunoglobulin formed by the respective Fc domains (or Fc moieties) of its two heavy chains. As used herein, the term “Fc domain” refers to a portion of a single immunoglobulin (Ig) heavy chain wherein the Fc domain does not comprise an Fv domain. As such, Fc domain can also be referred to as “Ig” or “IgG.” In some embodiments, an Fc domain begins in the hinge region just upstream of the papain cleavage site and ending at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In other embodiments, an Fc domain comprises a complete Fc domain (i.e., a hinge domain, a CH2 domain, and a CH3 domain). In one embodiment, an Fc domain comprises a hinge domain (or portion thereof) fused to a CH3 domain (or portion thereof). In another embodiment, an Fc domain comprises a CH2 domain (or portion thereof) fused to a CH3 domain (or portion thereof). In another embodiment, an Fc domain consists of a CH3 domain or portion thereof. In another embodiment, an Fc domain consists of a hinge domain (or portion thereof) and a CH3 domain (or portion thereof). In another embodiment, an Fc domain consists of a CH2 domain (or portion thereof) and a CH3 domain. In another embodiment, an Fc domain consists of a hinge domain (or portion thereof) and a CH2 domain (or portion thereof). In one embodiment, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. A human IgG1 constant region can be found at Uniprot P01857 and in Table 3 (i.e., SEQ ID NO: 33). The Fc domain of human IgG1 can be found in Table 3 (i.e., SEQ ID NO: 34). The Fc domain encompasses native Fc and Fc variant molecules. As with Fc variants and native Fc's, the term Fc domain includes molecules in monomeric or multimeric form, whether digested from whole antibody or produced by other means. The assignment of amino acid residue numbers to an Fc domain is in accordance with the definitions of Kabat. See, e.g., Sequences of Proteins of Immunological Interest (Table of Contents, Introduction and Constant Region Sequences sections), 5th edition, Bethesda, Md.:NIH vol. 1:647-723 (1991); Kabat et al., “Introduction” Sequences of Proteins of Immunological Interest, US Dept of Health and Human Services, NIH, 5th edition, Bethesda, Md. vol. 1:xiii-xcvi (1991); Chothia & Lesk, J. Mol. Biol. 196:901-917 (1987); Chothia et al., Nature 342:878-883 (1989), each of which is herein incorporated by reference for all purposes.

As set forth herein, it will be understood by one of ordinary skill in the art that any Fc domain may be modified such that it varies in amino acid sequence from the native Fc domain of a naturally occurring immunoglobulin molecule. In certain exemplary embodiments, the Fc domain has reduced effector function (e.g., FcγR binding).

The Fc domains of a polypeptide of the invention may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence.

Polypeptides derived from another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants necessarily have less than 100% sequence identity or similarity with the starting IL-2 molecule. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity.

In one embodiment, a polypeptide of the invention consists of, consists essentially of, or comprises an amino acid sequence selected from SEQ ID NOs: 2, 4, 6, 8, 12, 14, 16, 18, 20, 22, 24, 26, 28, 30, and 32. In an embodiment, a polypeptide includes an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence selected from SEQ ID NOs: 2, 4, 6, 8, 12, 14, 16, 18, 20, 22, 24, 26, 28, 30, and 32. In an embodiment, a polypeptide includes a contiguous amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous amino acid sequence selected from SEQ ID NOs: 2, 4, 6, 8, 12, 14, 16, 18, 20, 22, 24, 26, 28, 30, and 32. In an embodiment, a polypeptide includes an amino acid sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous amino acids of an amino acid sequence selected from SEQ ID NOs: 2, 4, 6, 8, 12, 14, 16, 18, 20, 22, 24, 26, 28, 30, and 32.

In an embodiment, the peptides of the invention are encoded by a nucleotide sequence. Nucleotide sequences of the invention can be useful for a number of applications, including: cloning, gene therapy, protein expression and purification, mutation introduction, DNA vaccination of a host in need thereof, antibody generation for, e.g., passive immunization, PCR, primer and probe generation, and the like. In an embodiment, the nucleotide sequence of the invention comprises, consists of, or consists essentially of, a nucleotide sequence selected from SEQ ID NOs: 1, 3, 5, 7, 11, 13, 15, 17, 19, 21, 23, 25, 27, 29, and 31. In an embodiment, a nucleotide sequence includes a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence set forth in SEQ ID NOs: 1, 3, 5, 7, 11, 13, 15, 17, 19, 21, 23, 25, 27, 29, and 31. In an embodiment, a nucleotide sequence includes a contiguous nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous nucleotide sequence set forth in SEQ ID NOs: 1, 3, 5, 7, 11, 13, 15, 17, 19, 21, 23, 25, 27, 29, and 31. In an embodiment, a nucleotide sequence includes a nucleotide sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous nucleotides of a nucleotide sequence set forth in SEQ ID NOs: 1, 3, 5, 7, 11, 13, 15, 17, 19, 21, 23, 25, 27, 29, and 31.

It will also be understood by one of ordinary skill in the art that the extended-PK IL-2 of the invention may be altered such that they vary in sequence from the naturally occurring or native sequences from which they were derived, while retaining the desirable activity of the native sequences. For example, nucleotide or amino acid substitutions leading to conservative substitutions or changes at “non-essential” amino acid residues may be made. Mutations may be introduced by standard techniques, such as site-directed mutagenesis and PCR-mediated mutagenesis.

The IL-2 and Fc molecules of the invention may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In another embodiment, a string of amino acids can be replaced with a structurally similar string that differs in order and/or composition of side chain family members. Alternatively, in another embodiment, mutations may be introduced randomly along all or part of a coding sequence, such as by saturation mutagenesis, and the resultant mutants can be incorporated into binding polypeptides of the invention and screened for their ability to bind to the desired target.

The term “ameliorating” refers to any therapeutically beneficial result in the treatment of a disease state, e.g., cancer, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” or “subject” or “patient” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

As used herein, the term “gly-ser polypeptide linker” refers to a peptide that consists of glycine and serine residues. An exemplary gly-ser polypeptide linker comprises the amino acid sequence Ser(Gly₄Ser)n. In one embodiment, n=1. In one embodiment, n=2. In another embodiment, n=3, i.e., Ser(Gly₄Ser)₃. In another embodiment, n=4, i.e., Ser(Gly₄Ser)₄. In another embodiment, n=5. In yet another embodiment, n=6. In another embodiment, n=7. In yet another embodiment, n=8. In another embodiment, n=9. In yet another embodiment, n=10. Another exemplary gly-ser polypeptide linker comprises the amino acid sequence (Gly₄Ser)n. In one embodiment, n=1. In one embodiment, n=2. In a preferred embodiment, n=3. In another embodiment, n=4. In another embodiment, n=5. In yet another embodiment, n=6. Another exemplary gly-ser polypeptide linker comprises the amino acid sequence (Gly₃Ser)n. In one embodiment, n=1. In one embodiment, n=2. In a preferred embodiment, n=3. In another embodiment, n=4. In another embodiment, n=5. In yet another embodiment, n=6.

As used herein, the terms “linked,” “fused”, or “fusion” are used interchangeably. These terms refer to the joining together of two more elements or components or domains, by whatever means including chemical conjugation or recombinant means. Methods of chemical conjugation (e.g., using heterobifunctional crosslinking agents) are known in the art.

As used herein, “half-life” refers to the time taken for the serum or plasma concentration of a polypeptide to reduce by 50%, in vivo, for example due to degradation and/or clearance or sequestration by natural mechanisms. The extended-PK IL-2 of the present invention is stabilized in vivo and its half-life increased by, e.g., fusion to an Fc region, through PEGylation, or by binding to serum albumin molecules (e.g., human serum albumin) which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound of the invention to a subject; collecting blood samples or other samples from said subject at regular intervals; determining the level or concentration of the amino acid sequence or compound of the invention in said blood sample; and calculating, from (a plot of) the data thus obtained, the time until the level or concentration of the amino acid sequence or compound of the invention has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

A “therapeutic antibody” is an antibody, fragment of an antibody, or construct that is derived from an antibody, and can bind to a cell-surface antigen on a target cell to cause a therapeutic effect. Such antibodies can be chimeric, humanized or fully human antibodies. Methods are known in the art for producing such antibodies. Such antibodies include single chain Fc fragments of antibodies, minibodies and diabodies. Any of the therapeutic antibodies known in the art to be useful for cancer therapy can be used in combination therapy with extended-PK IL-2 of the present invention. Therapeutic antibodies may be monoclonal antibodies or polyclonal antibodies. In preferred embodiments, the therapeutic antibodies target cancer antigens.

As used herein, “cancer antigen” refers to (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

As used herein, a “small molecule” is a molecule with a molecular weight below about 500 Daltons.

As used herein, “therapeutic protein” refers to any polypeptide, protein, protein variant, fusion protein and/or fragment thereof which may be administered to a subject as a medicament. An exemplary therapeutic protein is an interleukin, e.g., IL-7.

As used herein, “synergy” or “synergistic effect” with regard to an effect produced by two or more individual components refers to a phenomenon in which the total effect produced by these components, when utilized in combination, is greater than the sum of the individual effects of each component acting alone.

The term “sufficient amount” or “amount sufficient to” means an amount sufficient to produce a desired effect, e.g., an amount sufficient to reduce the size of a tumor.

The term “therapeutically effective amount” is an amount that is effective to ameliorate a symptom of a disease. A therapeutically effective amount can be a “prophylactically effective amount” as prophylaxis can be considered therapy.

The term “regression,” as used herein, does not necessarily imply 100% or complete regression. Rather, there are varying degrees of regression of which one of ordinary skill in the art recognizes as having a potential benefit or therapeutic effect. In this respect, the inventive methods can provide any amount of any level of regression of cancer in a mammal. Furthermore, the regression provided by the inventive method can include regression of one or more conditions or symptoms of the disease, e.g., cancer.

As used herein, “combination therapy” embraces administration of each agent or therapy in a sequential manner in a regiment that will provide beneficial effects of the combination, and co-administration of these agents or therapies in a substantially simultaneous manner, such as in a single capsule having a fixed ratio of these active agents or in multiple, separate capsules for each agent. Combination therapy also includes combinations where individual elements may be administered at different times and/or by different routes but which act in combination to provide a beneficial effect by co-action or pharmacokinetic and pharmacodynamics effect of each agent or tumor treatment approaches of the combination therapy. As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

It must be noted that, as used in the specification and the appended claims, the singular forms “a,” “an” and “the” include plural referents unless the context clearly dictates otherwise.

### Extended-PK IL-2

Interleukin-2 (IL-2) is a cytokine that induces proliferation of antigen-activated T cells and stimulates natural killer (NK) cells. The biological activity of IL-2 is mediated through a multi-subunit IL-2 receptor complex (IL-2R) of three polypeptide subunits that span the cell membrane: p55 (IL-2Rα, the alpha subunit, also known as CD25 in humans), p75 (IL-2Rβ, the beta subunit, also known as CD122 in humans) and p64 (IL-2Rγ, the gamma subunit, also known as CD132 in humans). T cell response to IL-2 depends on a variety of factors, including: (1) the concentration of IL-2; (2) the number of IL-2R molecules on the cell surface; and (3) the number of IL-2R occupied by IL-2 (i.e., the affinity of the binding interaction between IL-2 and IL-2R (Smith, “Cell Growth Signal Transduction is Quantal” In Receptor Activation by Antigens, Cytokines, Hormones, and Growth Factors 766:263-271, 1995)). The IL-2:IL-2R complex is internalized upon ligand binding and the different components undergo differential sorting. IL-2Rα is recycled to the cell surface, while IL-2 associated with the IL-2:IL-2Rβγ complex is routed to the lysosome and degraded. When administered as an intravenous (i.v.) bolus, IL-2 has a rapid systemic clearance (an initial clearance phase with a half-life of 12.9 minutes followed by a slower clearance phase with a half-life of 85 minutes) (Konrad et al., Cancer Res. 50:2009-2017, 1990).

Outcomes of systemic IL-2 administration in cancer patients are far from ideal. While 15 to 20 percent of patients respond objectively to high-dose IL-2, the great majority do not, and many suffer severe, life-threatening side effects, including nausea, confusion, hypotension, and septic shock. The severe toxicity associated with IL-2 treatment is largely attributable to the activity of natural killer (NK) cells. NK cells express the intermediate-affinity receptor, IL-2Rβγ_{c}, and thus are stimulated at nanomolar concentrations of IL-2, which do in fact result in patient sera during high-dose IL-2 therapy. Attempts to reduce serum concentration, and hence selectively stimulate IL-2Rαβγ_{c}-bearing cells, by reducing dose and adjusting dosing regimen have been attempted, and while less toxic, such treatments were also less efficacious.

The applicants recently discovered that the ability of IL-2 to control tumors in various cancer models could be substantially increased by attaching IL-2 to a pharmacokinetic modifying group. The resulting molecule, hereafter referred to as “extended-pharmacokinetic (PK) IL-2,” has a prolonged circulation half-life relative to free IL-2. The prolonged circulation half-life of extended-PK IL-2 permits in vivo serum IL-2 concentrations to be maintained within a therapeutic range, leading to the enhanced activation of many types of immune cells, including T cells. Because of its favorable pharmacokinetic profile, extended-PK IL-2 can be dosed less frequently and for longer periods of time when compared with unmodified IL-2. Extended-PK IL-2 is described in detail in International Patent Application No. PCT/US2013/042057, filed May 21, 2013, and claiming the benefit of priority to U.S. Provisional Patent Application No. 61/650,277, filed May 22, 2012. The entire contents of the foregoing applications are incorporated by reference herein.

A. IL-2 and Mutants Thereof.

In certain embodiments, the IL-2 portion of the extended-PK IL-2 is wild-type IL-2 (e.g., human IL-2 in its precursor form (SEQ ID NO: 30) or mature form (SEQ ID NO: 32)).

In some embodiments, the extended-PK IL-2 is mutated such that it has an altered affinity (e.g., a higher affinity) for the IL-2R alpha receptor compared with unmodified IL-2.

Site-directed mutagenesis was used to isolate IL-2 mutants that exhibit high affinity binding to CD25, i.e., IL-2Rα, as compared to wild-type IL-2. Increasing the affinity of IL-2 for IL-2Rα at the cell surface will increase receptor occupancy within a limited range of IL-2 concentration, as well as raise the local concentration of IL-2 at the cell surface.

In one embodiment, the invention features IL-2 mutants, which may be, but are not necessarily, substantially purified and which can function as high affinity CD25 binders. IL-2 is a T cell growth factor that induces proliferation of antigen-activated T cells and stimulation of NK cells. Exemplary IL-2 mutants of the present invention which are high affinity binders include those shown in FIG. 1, such as those with amino acid sequences set forth in SEQ ID NOs: 4, 20, 22, 24, 26, and 28. Further exemplary IL-2 mutants with increased affinity for CD25 are disclosed in U.S. Pat. No. 7,569,215, the contents of which are incorporated herein by reference. In one embodiment, the IL-2 mutant is does not bind to CD25, e.g., those with amino acid sequences set forth in SEQ ID NOs: 6 and 8.

IL-2 mutants include an amino acid sequence that is at least 80% identical to SEQ ID NO: 30 and that bind CD25. For example, an IL-2 mutant can have at least one mutation (e.g., a deletion, addition, or substitution of 1, 2, 3, 4, 5, 6, 7, 8, 9, 10, 11, 12, 13, 14, 15, 16, 17, 18, 19, 20, or more amino acid residues) that increases the affinity for the alpha subunit of the IL-2 receptor relative to wild-type IL-2. It should be understood that mutations identified in mouse IL-2 may be made at corresponding residues in full length human IL-2 (nucleic acid sequence (accession: NM000586) of SEQ ID NO: 29; amino acid sequence (accession: P60568) of SEQ ID NO: 30) or human IL-2 without the signal peptide (nucleic acid sequence of SEQ ID NO: 31; amino acid sequence of SEQ ID NO: 32). Accordingly, in preferred embodiments, the IL-2 moiety of the extended-PK IL-2 is human IL-2. In other embodiments, the IL-2 moiety of the extended-PK IL-2 is a mutant human IL-2.

IL-2 mutants can be at least or about 50%, at least or about 65%, at least or about 70%, at least or about 80%, at least or about 85%, at least or about 87%, at least or about 90%, at least or about 95%, at least or about 97%, at least or about 98%, or at least or about 99% identical to wild-type IL-2 (in its precursor form or, preferably, the mature form). The mutation can consist of a change in the number or content of amino acid residues. For example, the IL-2 mutants can have a greater or a lesser number of amino acid residues than wild-type IL-2. Alternatively, or in addition, IL-2 mutants can contain a substitution of one or more amino acid residues that are present in the wild-type IL-2.

By way of illustration, a polypeptide that includes an amino acid sequence that is at least 95% identical to a reference amino acid sequence of SEQ ID NO: 30 or 32 is a polypeptide that includes a sequence that is identical to the reference sequence except for the inclusion of up to five alterations of the reference amino acid of SEQ ID NO: 30 or 32. For example, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino (N—) or carboxy (C—) terminal positions of the reference amino acid sequence or anywhere between those terminal positions, interspersed either individually among residues in the reference sequence or in one or more contiguous groups within the reference sequence.

The substituted amino acid residue(s) can be, but are not necessarily, conservative substitutions, which typically include substitutions within the following groups: glycine, alanine; valine, isoleucine, leucine; aspartic acid, glutamic acid; asparagine, glutamine; serine, threonine; lysine, arginine; and phenylalanine, tyrosine. These mutations can be at amino acid residues that contact IL-2Rα.

In general, the polypeptides used in the practice of the instant invention will be synthetic, or produced by expression of a recombinant nucleic acid molecule. In the event the polypeptide is an extended-PK IL-2 (e.g., a fusion protein containing at least IL-2 and a heterologous polypeptide, such as a hexa-histidine tag or hemagglutinin tag or an Fc region or human serum albumin), it can be encoded by a hybrid nucleic acid molecule containing one sequence that encodes IL-2 and a second sequence that encodes all or part of the heterologous polypeptide.

The techniques that are required to make IL-2 mutants are routine in the art, and can be performed without resort to undue experimentation by one of ordinary skill in the art. For example, a mutation that consists of a substitution of one or more of the amino acid residues in IL-2 can be created using a PCR-assisted mutagenesis technique (e.g., as known in the art and/or described herein for the creation of IL-2 mutants). Mutations that consist of deletions or additions of amino acid residues to an IL-2 polypeptide can also be made with standard recombinant techniques. In the event of a deletion or addition, the nucleic acid molecule encoding IL-2 is simply digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

In addition to generating IL-2 mutants via expression of nucleic acid molecules that have been altered by recombinant molecular biological techniques, IL-2 mutants can be chemically synthesized. Chemically synthesized polypeptides are routinely generated by those of skill in the art.

As noted above, IL-2 can also be prepared as fusion or chimeric polypeptides that include IL-2 and a heterologous polypeptide (i.e., a polypeptide that is not IL-2). The heterologous polypeptide can increase the circulating half-life of the chimeric polypeptide in vivo, and may, therefore, further enhance the properties of IL-2. As discussed in further detail infra, the polypeptide that increases the circulating half-life may be a serum albumin, such as human serum albumin, or the Fc region of the IgG subclass of antibodies that lacks the IgG heavy chain variable region. The Fc region can include a mutation that inhibits effector functions such as complement fixation and Fc receptor binding.

In other embodiments, the chimeric polypeptide can include IL-2 and a polypeptide that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., Proc. Natl. Acad. Sci. USA 89:8145, 1992). In some embodiments, the chimeric polypeptide further comprises a C-terminal c-myc epitope tag.

Chimeric polypeptides can be constructed using no more than conventional molecular biological techniques, which are well within the ability of those of ordinary skill in the art to perform.

(i) Nucleic Acid Molecules Encoding IL-2 and Mutants Thereof

IL-2, either alone or as a part of a chimeric polypeptide, such as those described above, can be obtained by expression of a nucleic acid molecule. Thus, nucleic acid molecules encoding polypeptides containing IL-2 or an IL-2 mutant are considered within the scope of the invention, such as those with nucleic acid sequences set forth in SEQ ID NOs: 1, 3, 5, 7, 11, 13, 15, 17, 19, 21, 23, 25, 27, 29, and 31. Just as IL-2 mutants can be described in terms of their identity with wild-type IL-2, the nucleic acid molecules encoding them will necessarily have a certain identity with those that encode wild-type IL-2. For example, the nucleic acid molecule encoding an IL-2 mutant can be at least 50%, at least 65%, preferably at least 75%, more preferably at least 85%, and most preferably at least 95% (e.g., 99%) identical to the nucleic acid encoding full length wild-type IL-2 (e.g., SEQ ID NO: 29) or wild-type IL-2 without the signal peptide (e.g., SEQ ID NO: 31).

The nucleic acid molecules of the invention can contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

The nucleic acid molecules are not limited to sequences that encode polypeptides; some or all of the non-coding sequences that lie upstream or downstream from a coding sequence (e.g., the coding sequence of IL-2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The isolated nucleic acid molecules of the invention can include fragments not found as such in the natural state. Thus, the invention encompasses recombinant molecules, such as those in which a nucleic acid sequence (for example, a sequence encoding an IL-2 mutant) is incorporated into a vector (e.g., a plasmid or viral vector) or into the genome of a heterologous cell (or the genome of a homologous cell, at a position other than the natural chromosomal location).

As described above, IL-2 mutants of the invention may exist as a part of a chimeric polypeptide. In addition to, or in place of, the heterologous polypeptides described above, a nucleic acid molecule of the invention can contain sequences encoding a “marker” or “reporter.” Examples of marker or reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-hosphotransferase (HPH), thymidine kinase (TK), lacz (encoding β-galactosidase), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter.

The nucleic acid molecules of the invention can be obtained by introducing a mutation into IL-2-encoding DNA obtained from any biological cell, such as the cell of a mammal. Thus, the nucleic acids of the invention (and the polypeptides they encode) can be those of a mouse, rat, guinea pig, cow, sheep, horse, pig, rabbit, monkey, baboon, dog, or cat. Typically, the nucleic acid molecules will be those of a human.

(ii) Expression of IL-2 and Mutants Thereof

The nucleic acid molecules described above can be contained within a vector that is capable of directing their expression in, for example, a cell that has been transduced with the vector. Accordingly, in addition to IL-2 and mutants thereof, expression vectors containing a nucleic acid molecule encoding IL-2 or an IL-2 mutant and cells transfected with these vectors are among the preferred embodiments.

Vectors suitable for use in the present invention include T7-based vectors for use in bacteria (see, for example, Rosenberg et al., Gene 56:125, 1987), the pMSXND expression vector for use in mammalian cells (Lee and Nathans, J. Biol. Chem. 263:3521, 1988), and baculovirus-derived vectors (for example the expression vector pBacPAK9 from Clontech, Palo Alto, Calif.) for use in insect cells. The nucleic acid inserts, which encode the polypeptide of interest in such vectors, can be operably linked to a promoter, which is selected based on, for example, the cell type in which expression is sought. For example, a T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neo^{r}) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the invention include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes an IL-2 mutant are also features of the invention. A cell of the invention is a transfected cell, i.e., a cell into which a nucleic acid molecule, for example a nucleic acid molecule encoding an IL-2 mutant, has been introduced by means of recombinant DNA techniques. The progeny of such a cell are also considered within the scope of the invention.

The precise components of the expression system are not critical. For example, an IL-2 mutant can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., as therapeutic agents, as described herein.

B. Extended-PK Groups

As described supra, IL-2 or mutant IL-2 is fused to an extended-PK group, which increases circulation half-life. Non-limiting examples of extended-PK groups are described infra. It should be understood that other PK groups that increase the circulation half-life of IL-2, or variants thereof, are also applicable to the present invention. In a preferred embodiment, the extended-PK group is a Fc domain.

In some embodiments, the serum half-life of extended-PK IL-2 is increased relative to IL-2 alone (i.e., IL-2 not fused to an extended-PK group). In certain embodiments, the serum half-life of extended-PK IL-2 is at least 20, 40, 60, 80, 100, 120, 150, 180, 200, 400, 600, 800, or 1000% longer relative to the serum half-life of IL-2 alone. In other embodiments, the serum half-life of the extended-PK IL-2 is at least 1.5-fold, 2-fold, 2.5-fold, 3-fold, 3.5 fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of IL-2 alone. In some embodiments, the serum half-life of the extended-PK IL-2 is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

(i) Fc Domains

In some embodiments, an extended-PK IL-2 includes an Fc domain, such as that with an amino acid sequences set forth in SEQ ID NO: 34. It will be understood by those in the art that epitope tags corresponding to 6× his tag on these extended-PK IL-2 with Fc domains are optional. The Fc domain does not contain a variable region that binds to antigen. Fc domains useful for producing the extended-PK IL-2 of the present invention may be obtained from a number of different sources. In preferred embodiments, an Fc domain of the extended-PK IL-2 is derived from a human immunoglobulin. In a preferred embodiment, the Fc domain is from a human IgG1 constant region (SEQ ID NO: 33). The Fc domain of human IgG1 is set forth in SEQ ID NO: 34. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species. Moreover, the Fc domain or portion thereof may be derived from any immunoglobulin class, including IgM, IgG, IgD, IgA, and IgE, and any immunoglobulin isotype, including IgG1, IgG2, IgG3, and IgG4.

In some aspects, an extended-PK IL-2 includes a mutant Fc domain, e.g., an Fc domain with reduced effector function (e.g., reduced binding to Fc gamma receptors, antibody dependent cell-mediated cytotoxicity, and/or reduced complement dependent cytotoxicity). In some aspects, an extended-PK IL-2 includes a mutant, IgG1 Fc domain. In some aspects, a mutant Fc domain comprises one or more mutations in the hinge, CH2, and/or CH3 domains. In some aspects, a mutant Fc domain includes a D265A mutation.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or portions thereof) can be derived from these sequences using art recognized techniques. The genetic material obtained using any of the foregoing methods may then be altered or synthesized to obtain polypeptides of the present invention. It will further be appreciated that the scope of this invention encompasses alleles, variants and mutations of constant region DNA sequences.

Fc domain sequences can be cloned, e.g., using the polymerase chain reaction and primers which are selected to amplify the domain of interest. To clone an Fc domain sequence from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, Calif. (1990); Ho et al. 1989. Gene 77:51; Horton et al. 1993. Methods Enzymol. 217:270). PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7:1509); rapid amplification of cDNA ends (Ruberti, F. et al. 1994. J. Immunol. Methods 173:33); antibody leader sequences (Larrick et al. Biochem Biophys Res Commun 1989; 160:1250). The cloning of antibody sequences is further described in Newman et al., U.S. Pat. No. 5,658,570, filed Jan. 25, 1995, which is herein incorporated by reference.

Extended-PK IL-2 of the invention may comprise one or more Fc domains (e.g., 2, 3, 4, 5, 6, 7, 8, 9, 10, or more Fc domains). In one embodiment, the Fc domains may be of different types. In one embodiment, at least one Fc domain present in the extended-PK IL-2 comprises a hinge domain or portion thereof. In another embodiment, the extended-PK IL-2 of the invention comprises at least one Fc domain which comprises at least one CH2 domain or portion thereof. In another embodiment, the extended-PK IL-2 of the invention comprises at least one Fc domain which comprises at least one CH3 domain or portion thereof. In another embodiment, the extended-PK IL-2 of the invention comprises at least one Fc domain which comprises at least one CH4 domain or portion thereof. In another embodiment, the extended-PK IL-2 of the invention comprises at least one Fc domain which comprises at least one hinge domain or portion thereof and at least one CH2 domain or portion thereof (e.g, in the hinge-CH2 orientation). In another embodiment, the extended-PK IL-2 of the invention comprises at least one Fc domain which comprises at least one CH2 domain or portion thereof and at least one CH3 domain or portion thereof (e.g, in the CH2-CH3 orientation). In another embodiment, the extended-PK IL-2 of the invention comprises at least one Fc domain comprising at least one hinge domain or portion thereof, at least one CH2 domain or portion thereof, and least one CH3 domain or portion thereof, for example in the orientation hinge-CH2-CH3, hinge-CH3-CH2, or CH2-CH3-hinge.

In certain embodiments, extended-PK IL-2 comprises at least one complete Fc region derived from one or more immunoglobulin heavy chains (e.g., an Fc domain including hinge, CH2, and CH3 domains, although these need not be derived from the same antibody). In other embodiments, extended-PK IL-2 comprises at least two complete Fc domains derived from one or more immunoglobulin heavy chains. In preferred embodiments, the complete Fc domain is derived from a human IgG immunoglobulin heavy chain (e.g., human IgG1).

In another embodiment, the extended-PK IL-2 of the invention comprises at least one Fc domain comprising a complete CH3 domain. In another embodiment, the extended-PK IL-2 of the invention comprises at least one Fc domain comprising a complete CH2 domain. In another embodiment, the extended-PK IL-2 of the invention comprises at least one Fc domain comprising at least a CH3 domain, and at least one of a hinge region, and a CH2 domain. In one embodiment, the extended-PK IL-2 of the invention comprises at least one Fc domain comprising a hinge and a CH3 domain. In another embodiment, the extended-PK IL-2 of the invention comprises at least one Fc domain comprising a hinge, a CH2, and a CH3 domain. In preferred embodiments, the Fc domain is derived from a human IgG immunoglobulin heavy chain (e.g., human IgG1).

The constant region domains or portions thereof making up an Fc domain of the extended-PK IL-2 of the invention may be derived from different immunoglobulin molecules. For example, a polypeptide of the invention may comprise a CH2 domain or portion thereof derived from an IgG1 molecule and a CH3 region or portion thereof derived from an IgG3 molecule. In another example, the extended-PK IL-2 can comprise an Fc domain comprising a hinge domain derived, in part, from an IgG1 molecule and, in part, from an IgG3 molecule. As set forth herein, it will be understood by one of ordinary skill in the art that an Fc domain may be altered such that it varies in amino acid sequence from a naturally occurring antibody molecule.

In one embodiment, the extended-PK IL-2 of the invention lacks one or more constant region domains of a complete Fc region, i.e., they are partially or entirely deleted. In certain embodiments, the extended-PK IL-2 of the invention will lack an entire CH2 domain. In certain embodiments, the extended-PK IL-2 of the invention comprise CH2 domain-deleted Fc regions derived from a vector (e.g., from IDEC Pharmaceuticals, San Diego) encoding an IgG1 human constant region domain (see, e.g., WO02/060955A2 and WO02/096948A2). This exemplary vector is engineered to delete the CH2 domain and provide a synthetic vector expressing a domain-deleted IgG1 constant region. It will be noted that these exemplary constructs are preferably engineered to fuse a binding CH3 domain directly to a hinge region of the respective Fc domain.

In other constructs it may be desirable to provide a peptide spacer between one or more constituent Fc domains. For example, a peptide spacer may be placed between a hinge region and a CH2 domain and/or between a CH2 and a CH3 domain. For example, compatible constructs could be expressed wherein the CH2 domain has been deleted and the remaining CH3 domain (synthetic or unsynthetic) is joined to the hinge region with a 1-20, 1-10, or 1-5 amino acid peptide spacer. Such a peptide spacer may be added, for instance, to ensure that the regulatory elements of the constant region domain remain free and accessible or that the hinge region remains flexible. Preferably, any linker peptide compatible with the instant invention will be relatively non-immunogenic and not prevent proper folding of the Fc.

(ii) Changes to Fc Amino Acids

In certain embodiments, an Fc domain employed in the extended-PK IL-2 of the invention is altered or modified, e.g., by amino acid mutation (e.g., addition, deletion, or substitution). As used herein, the term “Fc domain variant” refers to an Fc domain having at least one amino acid modification, such as an amino acid substitution, as compared to the wild-type Fc from which the Fc domain is derived. For example, wherein the Fc domain is derived from a human IgG1 antibody, a variant comprises at least one amino acid mutation (e.g., substitution) as compared to a wild type amino acid at the corresponding position of the human IgG1 Fc region.

In one embodiment, the Fc variant comprises a substitution at an amino acid position located in a hinge domain or portion thereof. In another embodiment, the Fc variant comprises a substitution at an amino acid position located in a CH2 domain or portion thereof. In another embodiment, the Fc variant comprises a substitution at an amino acid position located in a CH3 domain or portion thereof. In another embodiment, the Fc variant comprises a substitution at an amino acid position located in a CH4 domain or portion thereof.

In certain embodiments, the extended-PK IL-2 of the invention comprise an Fc variant comprising more than one amino acid substitution. The extended-PK IL-2 of the invention may comprise, for example, 2, 3, 4, 5, 6, 7, 8, 9, 10 or more amino acid substitutions. Preferably, the amino acid substitutions are spatially positioned from each other by an interval of at least 1 amino acid position or more, for example, at least 2, 3, 4, 5, 6, 7, 8, 9, or 10 amino acid positions or more. More preferably, the engineered amino acids are spatially positioned apart from each other by an interval of at least 5, 10, 15, 20, or 25 amino acid positions or more.

In some aspects, an Fc domain includes changes in the region between amino acids 234-238, including the sequence LLGGP at the beginning of the CH2 domain. In some aspects, an Fc variant alters Fc mediated effector function, particularly ADCC, and/or decrease binding avidity for Fc receptors. In some aspects, sequence changes closer to the CH2-CH3 junction, at positions such as K322 or P331 can eliminate complement mediated cytotoxicity and/or alter avidity for FcR binding. In some aspects, an Fc domain incorporates changes at residues P238 and P331, e.g., changing the wild type prolines at these positions to serine. In some aspects, alterations in the hinge region at one or more of the three hinge cysteines, to encode CCC, SCC, SSC, SCS, or SSS at these residues can also affect FcR binding and molecular homogeneity, e.g., by elimination of unpaired cysteines that may destabilize the folded protein.

Other amino acid mutations in the Fc domain are contemplated to reduce binding to the Fc gamma receptor and Fc gamma receptor subtypes. For example, mutations at positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 322, 324, 327, 329, 330, 331, 333, 334, 335, 337, 338, 340, 356, 360, 373, 376, 378, 379, 382, 388, 389, 398, 414, 416, 419, 430, 434, 435, 437, 438 or 439 of the Fc region can alter binding as described in U.S. Pat. No. 6,737,056, issued May 18, 2004, incorporated herein by reference in its entirety. This patent reported that changing Pro331 in IgG3 to Ser resulted in six fold lower affinity as compared to unmutated IgG3, indicating the involvement of Pro331 in Fc gamma RI binding. In addition, amino acid modifications at positions 234, 235, 236, and 237, 297, 318, 320 and 322 are disclosed as potentially altering receptor binding affinity in U.S. Pat. No. 5,624,821, issued Apr. 29, 1997 and incorporated herein by reference in its entirety.

Further mutations contemplated for use include, e.g., those described in U.S. Pat. App. Pub. No. 2006/0235208, published Oct. 19, 2006 and incorporated herein by reference in its entirety. This publication describes Fc variants that exhibit reduced binding to Fc gamma receptors, reduced antibody dependent cell-mediated cytotoxicity, or reduced complement dependent cytotoxicity, that comprise at least one amino acid modification in the Fc region, including 232G, 234G, 234H, 235D, 235G, 235H, 236I, 236N, 236P, 236R, 237K, 237L, 237N, 237P, 238K, 239R, 265G, 265A, 267R, 269R, 270H, 297S, 299A, 299I, 299V, 325A, 325L, 327R, 328R, 329K, 330I, 330L, 330N, 330P, 330R, and 331L (numbering is according to the EU index), as well as double mutants 236R/237K, 236R/325L, 236R/328R, 237K/325L, 237K/328R, 325L/328R, 235G/236R, 267R/269R, 234G/235G, 236R/237K/325L, 236R/325L/328R, 235G/236R/237K, and 237K/325L/328R. Other mutations contemplated for use as described in this publication include 227G, 234D, 234E, 234G, 234I, 234Y, 235D, 235I, 235S, 236S, 239D, 246H, 255Y, 258H, 260H, 264I, 267D, 267E, 268D, 268E, 272H, 272I, 272R, 281D, 282G, 283H, 284E, 293R, 295E, 304T, 324G, 324I, 327D, 327A, 328A, 328D, 328E, 328F, 328I, 328M, 328N, 328Q, 328T, 328V, 328Y, 330I, 330L, 330Y, 332D, 332E, 335D, an insertion of G between positions 235 and 236, an insertion of A between positions 235 and 236, an insertion of S between positions 235 and 236, an insertion of T between positions 235 and 236, an insertion of N between positions 235 and 236, an insertion of D between positions 235 and 236, an insertion of V between positions 235 and 236, an insertion of L between positions 235 and 236, an insertion of G between positions 235 and 236, an insertion of A between positions 235 and 236, an insertion of S between positions 235 and 236, an insertion of T between positions 235 and 236, an insertion of N between positions 235 and 236, an insertion of D between positions 235 and 236, an insertion of V between positions 235 and 236, an insertion of L between positions 235 and 236, an insertion of G between positions 297 and 298, an insertion of A between positions 297 and 298, an insertion of S between positions 297 and 298, an insertion of D between positions 297 and 298, an insertion of G between positions 326 and 327, an insertion of A between positions 326 and 327, an insertion of T between positions 326 and 327, an insertion of D between positions 326 and 327, and an insertion of E between positions 326 and 327 (numbering is according to the EU index). Additionally, mutations described in U.S. Pat. App. Pub. No. 2006/0235208 include 227G/332E, 234D/332E, 234E/332E, 234Y/332E, 234I/332E, 234G/332E, 235I/332E, 235S/332E, 235D/332E, 235E/332E, 236S/332E, 236A/332E, 236S/332D, 236A/332D, 239D/268E, 246H/332E, 255Y/332E, 258H/332E, 260H/332E, 264I/332E, 267E/332E, 267D/332E, 268D/332D, 268E/332D, 268E/332E, 268D/332E, 268E/330Y, 268D/330Y, 272R/332E, 272H/332E, 283H/332E, 284E/332E, 293R/332E, 295E/332E, 304T/332E, 324I/332E, 324G/332E, 324I/332D, 324G/332D, 327D/332E, 328A/332E, 328T/332E, 328V/332E, 328I/332E, 328F/332E, 328Y/332E, 328M/332E, 328D/332E, 328E/332E, 328N/332E, 328Q/332E, 328A/332D, 328T/332D, 328V/332D, 328I/332D, 328F/332D, 328Y/332D, 328M/332D, 328D/332D, 328E/332D, 328N/332D, 328Q/332D, 330L/332E, 330Y/332E, 330I/332E, 332D/330Y, 335D/332E, 239D/332E, 239D/332E/330Y, 239D/332E/330L, 239D/332E/330I, 239D/332E/268E, 239D/332E/268D, 239D/332E/327D, 239D/332E/284E, 239D/268E/330Y, 239D/332E/268E/330Y, 239D/332E/327A, 239D/332E/268E/327A, 239D/332E/330Y/327A, 332E/330Y/268 E/327A, 239D/332E/268E/330Y/327A, Insert G>297-298/332E, Insert A>297-298/332E, Insert S>297-298/332E, Insert D>297-298/332E, Insert G>326-327/332E, Insert A>326-327/332E, Insert T>326-327/332E, Insert D>326-327/332E, Insert E>326-327/332E, Insert G>235-236/332E, Insert A>235-236/332E, Insert S>235-236/332E, Insert T>235-236/332E, Insert N>235-236/332E, Insert D>235-236/332E, Insert V>235-236/332E, Insert L>235-236/332E, Insert G>235-236/332D, Insert A>235-236/332D, Insert S>235-236/332D, Insert T>235-236/332D, Insert N>235-236/332D, Insert D>235-236/332D, Insert V>235-236/332D, and Insert L>235-236/332D (numbering according to the EU index) are contemplated for use. The mutant L234A/L235A is described, e.g., in U.S. Pat. App. Pub. No. 2003/0108548, published Jun. 12, 2003 and incorporated herein by reference in its entirety. In embodiments, the described modifications are included either individually or in combination. In a preferred embodiment, the mutation is D265A in human IgG1.

In certain embodiments, the extended-PK IL-2 of the invention comprises an amino acid substitution to an Fc domain which alters the antigen-independent effector functions of the antibody, in particular the circulating half-life of the antibody.

In other embodiments, the extended-PK IL-2 of the invention comprises an Fc variant comprising an amino acid substitution which alters the antigen-dependent effector functions of the polypeptide, in particular ADCC or complement activation, e.g., as compared to a wild type Fc region. Such extended-PK IL-2 exhibit decreased binding to FcR gamma when compared to wild-type polypeptides and, therefore, mediate reduced effector function. Fc variants with decreased FcR gamma binding affinity are expected to reduce effector function, and such molecules are also useful, for example, for treatment of conditions in which target cell destruction is undesirable, e.g., where normal cells may express target molecules, or where chronic administration of the polypeptide might result in unwanted immune system activation.

In one embodiment, the extended-PK IL-2 exhibits altered binding to an activating FcγR (e.g. FcγI, FcγIIa, or FcγRIIIa). In another embodiment, the extended-PK IL-2 exhibits altered binding affinity to an inhibitory FcγR (e.g. FcγRIIb). Exemplary amino acid substitutions which altered FcR or complement binding activity are disclosed in International PCT Publication No. WO05/063815 which is incorporated by reference herein.

The extended-PK IL-2 of the invention may also comprise an amino acid substitution which alters the glycosylation of the extended-PK IL-2. For example, the Fc domain of the extended-PK IL-2 may comprise an Fc domain having a mutation leading to reduced glycosylation (e.g., N- or O-linked glycosylation) or may comprise an altered glycoform of the wild-type Fc domain (e.g., a low fucose or fucose-free glycan). In another embodiment, the extended-PK IL-2 has an amino acid substitution near or within a glycosylation motif, for example, an N-linked glycosylation motif that contains the amino acid sequence NXT or NXS. Exemplary amino acid substitutions which reduce or alter glycosylation are disclosed in WO05/018572 and US2007/0111281, which are incorporated by reference herein.

In other embodiments, the extended-PK IL-2 of the invention comprises at least one Fc domain having engineered cysteine residue or analog thereof which is located at the solvent-exposed surface. In preferred embodiments, the extended-PK IL-2 of the invention comprise an Fc domain comprising at least one engineered free cysteine residue or analog thereof that is substantially free of disulfide bonding with a second cysteine residue. Any of the above engineered cysteine residues or analogs thereof may subsequently be conjugated to a functional domain using art-recognized techniques (e.g., conjugated with a thiol-reactive heterobifunctional linker).

In one embodiment, the extended-PK IL-2 of the invention may comprise a genetically fused Fc domain having two or more of its constituent Fc domains independently selected from the Fc domains described herein. In one embodiment, the Fc domains are the same. In another embodiment, at least two of the Fc domains are different. For example, the Fc domains of the extended-PK IL-2 of the invention comprise the same number of amino acid residues or they may differ in length by one or more amino acid residues (e.g., by about 5 amino acid residues (e.g., 1, 2, 3, 4, or 5 amino acid residues), about 10 residues, about 15 residues, about 20 residues, about 30 residues, about 40 residues, or about 50 residues). In yet other embodiments, the Fc domains of the extended-PK IL-2 of the invention may differ in sequence at one or more amino acid positions. For example, at least two of the Fc domains may differ at about 5 amino acid positions (e.g., 1, 2, 3, 4, or 5 amino acid positions), about 10 positions, about 15 positions, about 20 positions, about 30 positions, about 40 positions, or about 50 positions).

(iii) PEGylation

In some embodiments, an extended-PK IL-2 of the present invention includes a polyethylene glycol (PEG) domain. PEGylation is well known in the art to confer increased circulation half-life to proteins. Methods of PEGylation are well known and disclosed in, e.g., U.S. Pat. No. 7,610,156, U.S. Pat. No. 7,847,062, all of which are hereby incorporated by reference.

PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ₋₁CH₂CH₂OH, where n is 20 to 2300 and X is H or a terminal modification, e.g., a C₁₋₄ alkyl. In one embodiment, the PEG of the invention terminates on one end with hydroxy or methoxy, i.e., X is H or CH₃ (“methoxy PEG”). PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-A 0 473 084 and U.S. Pat. No. 5,932,462, both of which are hereby incorporated by reference. One form of PEGs includes two PEG side-chains (PEG2) linked via the primary amino groups of a lysine (Monfardini et al., Bioconjugate Chem 1995; 6:62-9).

In one embodiment, pegylated IL-2 is produced by site-directed pegylation, particularly by conjugation of PEG to a cysteine moiety at the N- or C-terminus. A PEG moiety may also be attached by other chemistry, including by conjugation to amines.

PEG conjugation to peptides or proteins generally involves the activation of PEG and coupling of the activated PEG-intermediates directly to target proteins/peptides or to a linker, which is subsequently activated and coupled to target proteins/peptides (see Abuchowski et al., JBC 1977; 252:3571 and JBC 1977; 252:3582, and Harris et. al., in: Poly(ethylene glycol) Chemistry: Biotechnical and Biomedical Applications; (J. M. Harris ed.) Plenum Press: New York, 1992; Chap. 21 and 22).

A variety of molecular mass forms of PEG can be selected, e.g., from about 1,000 Daltons (Da) to 100,000 Da (n is 20 to 2300), for conjugating to IL-2. The number of repeating units “n” in the PEG is approximated for the molecular mass described in Daltons. It is preferred that the combined molecular mass of PEG on an activated linker is suitable for pharmaceutical use. Thus, in one embodiment, the molecular mass of the PEG molecules does not exceed 100,000 Da. For example, if three PEG molecules are attached to a linker, where each PEG molecule has the same molecular mass of 12,000 Da (each n is about 270), then the total molecular mass of PEG on the linker is about 36,000 Da (total n is about 820). The molecular masses of the PEG attached to the linker can also be different, e.g., of three molecules on a linker two PEG molecules can be 5,000 Da each (each n is about 110) and one PEG molecule can be 12,000 Da (n is about 270).

One skilled in the art can select a suitable molecular mass for PEG, e.g., based on how the pegylated IL-2 will be used therapeutically, the desired dosage, circulation time, resistance to proteolysis, immunogenicity, and other considerations. For a discussion of PEG and its use to enhance the properties of proteins, see N. V. Katre, Advanced Drug Delivery Reviews 1993; 10:91-114.

In one embodiment of the invention, PEG molecules may be activated to react with amino groups on IL-2 such as with lysines (Bencham C. O. et al., Anal. Biochem., 131, 25 (1983); Veronese, F. M. et al., Appl. Biochem., 11, 141 (1985); Zalipsky, S. et al., Polymeric Drugs and Drug Delivery Systems, adrs 9-110 ACS Symposium Series 469 (1999); Zalipsky, S. et al., Europ. Polym. J., 19, 1177-1183 (1983); Delgado, C. et al., Biotechnology and Applied Biochemistry, 12, 119-128 (1990)).

In one embodiment, carbonate esters of PEG are used to form the PEG-IL-2 conjugates. N,N′-disuccinimidylcarbonate (DSC) may be used in the reaction with PEG to form active mixed PEG-succinimidyl carbonate that may be subsequently reacted with a nucleophilic group of a linker or an amino group of IL-2 (see U.S. Pat. No. 5,281,698 and U.S. Pat. No. 5,932,462). In a similar type of reaction, 1,1′-(dibenzotriazolyl)carbonate and di-(2-pyridyl)carbonate may be reacted with PEG to form PEG-benzotriazolyl and PEG-pyridyl mixed carbonate (U.S. Pat. No. 5,382,657), respectively.

Pegylation of IL-2 can be performed according to the methods of the state of the art, for example by reaction of IL-2 with electrophilically active PEGs (Shearwater Corp., USA, www.shearwatercorp.com). Preferred PEG reagents of the present invention are, e.g., N-hydroxysuccinimidyl propionates (PEG-SPA), butanoates (PEG-SBA), PEG-succinimidyl propionate or branched N-hydroxysuccinimides such as mPEG2-NHS (Monfardini, C., et al., Bioconjugate Chem. 6 (1995) 62-69).

In another embodiment, PEG molecules may be coupled to sulfhydryl groups on IL-2 (Sartore, L., et al., Appl. Biochem. Biotechnol., 27, 45 (1991); Morpurgo et al., Biocon. Chem., 7, 363-368 (1996); Goodson et al., Bio/Technology (1990) 8, 343; U.S. Pat. No. 5,766,897). U.S. Pat. No. 6,610,281 and U.S. Pat. No. 5,766,897 describe exemplary reactive PEG species that may be coupled to sulfhydryl groups.

In some embodiments where PEG molecules are conjugated to cysteine residues on IL-2 the cysteine residues are native to IL-2 whereas in other embodiments, one or more cysteine residues are engineered into IL-2. Mutations may be introduced into the coding sequence of IL-2 to generate cysteine residues. This might be achieved, for example, by mutating one or more amino acid residues to cysteine. Preferred amino acids for mutating to a cysteine residue include serine, threonine, alanine and other hydrophilic residues. Preferably, the residue to be mutated to cysteine is a surface-exposed residue. Algorithms are well-known in the art for predicting surface accessibility of residues based on primary sequence or a protein.

In another embodiment, pegylated IL-2 comprise one or more PEG molecules covalently attached to a linker.

In one embodiment, IL-2 is pegylated at the C-terminus. In a specific embodiment, a protein is pegylated at the C-terminus by the introduction of C-terminal azido-methionine and the subsequent conjugation of a methyl-PEG-triarylphosphine compound via the Staudinger reaction. This C-terminal conjugation method is described in Cazalis et al., C-Terminal Site-Specific PEGylation of a Truncated Thrombomodulin Mutant with Retention of Full Bioactivity, Bioconjug Chem. 2004; 15(5):1005-1009.

Monopegylation of IL-2 can also be achieved according to the general methods described in WO 94/01451. WO 94/01451 describes a method for preparing a recombinant polypeptide with a modified terminal amino acid alpha-carbon reactive group. The steps of the method involve forming the recombinant polypeptide and protecting it with one or more biologically added protecting groups at the N-terminal alpha-amine and C-terminal alpha-carboxyl. The polypeptide can then be reacted with chemical protecting agents to selectively protect reactive side chain groups and thereby prevent side chain groups from being modified. The polypeptide is then cleaved with a cleavage reagent specific for the biological protecting group to form an unprotected terminal amino acid alpha-carbon reactive group. The unprotected terminal amino acid alpha-carbon reactive group is modified with a chemical modifying agent. The side chain protected terminally modified single copy polypeptide is then deprotected at the side chain groups to form a terminally modified recombinant single copy polypeptide. The number and sequence of steps in the method can be varied to achieve selective modification at the N- and/or C-terminal amino acid of the polypeptide.

The ratio of IL-2 to activated PEG in the conjugation reaction can be from about 1:0.5 to 1:50, between from about 1:1 to 1:30, or from about 1:5 to 1:15. Various aqueous buffers can be used to catalyze the covalent addition of PEG to IL-2, or variants thereof. In one embodiment, the pH of a buffer used is from about 7.0 to 9.0. In another embodiment, the pH is in a slightly basic range, e.g., from about 7.5 to 8.5. Buffers having a pKa close to neutral pH range may be used, e.g., phosphate buffer.

Conventional separation and purification techniques known in the art can be used to purify PEGylated IL-2, such as size exclusion (e.g. gel filtration) and ion exchange chromatography. Products may also be separated using SDS-PAGE. Products that may be separated include mono-, di-, tri-poly- and un-pegylated IL-2 as well as free PEG. The percentage of mono-PEG conjugates can be controlled by pooling broader fractions around the elution peak to increase the percentage of mono-PEG in the composition.

In one embodiment, PEGylated IL-2 of the invention contain one, two or more PEG moieties. In one embodiment, the PEG moiety(ies) are bound to an amino acid residue which is on the surface of the protein and/or away from the surface that contacts CD25. In one embodiment, the combined or total molecular mass of PEG in PEG-IL-2 is from about 3,000 Da to 60,000 Da, optionally from about 10,000 Da to 36,000 Da. In one embodiment, PEG in pegylated IL-2 is a substantially linear, straight-chain PEG.

In one embodiment, pegylated IL-2 of the invention will preferably retain at least 25%, 50%, 60%, 70%, 80%, 85%, 90%, 95% or 100% of the biological activity associated with the unmodified protein. In one embodiment, biological activity refers to the ability to bind CD25.

The serum clearance rate of PEG-modified IL-2 may be decreased by about 10%, 20%, 30%, 40%, 50%, 60%, 70%, 80%, or even 90%, relative to the clearance rate of the unmodified IL-2. PEG-modified IL-2 may have a circulation half-life (t_{1/2}) which is enhanced relative to the half-life of unmodified IL-2. The half-life of PEG-IL-2, or variants thereof, may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of unmodified IL-2. In some embodiments, the protein half-life is determined in vitro, such as in a buffered saline solution or in serum. In other embodiments, the protein half-life is an in vivo circulation half-life, such as the half-life of the protein in the serum or other bodily fluid of an animal.

(iv) Serum Albumin

In some embodiments, the extended-PK moiety is serum albumin (e.g., HSA), or a variant of fragment thereof.

Suitable albumins for use as the extended-PK moiety can be from any species, e.g., human, primate, rodent, bovine, equine, donkey, rabbit, goat, sheep, dog, chicken, or pig. In a preferred embodiment, the albumin is a serum albumin, such as human serum albumin (HSA) (precursor HSA, SEQ ID NO: 35; mature HSA, SEQ ID NO: 36).

The albumin, or a variant or fragment thereof, generally has a sequence identity to the sequence of wild-type HSA as set forth in SEQ ID NO: 35 or 36 of at least 50%, such as at least 60%, at least 70%, at least 80%, at least 85%, at least 90%, at least 95%, at least 96%, at least 97%, at least 98%, or at least 99%.

In some embodiments, the number of alterations, e.g., substitutions, insertions, or deletions, in the albumin variants is 1-20, e.g., 1-10 and 1-5, such as 1, 2, 3, 4, 5, 6, 7, 8, 9 or 10 alterations compared to the corresponding wild-type albumin (e.g., HSA) (SEQ ID NO: 35 or 36).

In addition to wild-type albumin, albumin variants with increased serum half-life relative to the wild-type albumin, and/or that increase the serum half-life of molecules they are fused or conjugated to, are considered applicable as a PK moiety for use in the extended-PK/IL-2 fusions. Some natural variants of albumin also exhibit increased serum half-life, and are suitable for use as a PK moiety. Such natural HSA variants with increased serum half-life are known in the art, such as E501K, E570K (Iwao et al. 2007, B.B.A. Proteins and Proteomics 1774, 1582-90), E505K (Gallino et al., supra), K536E, K574N (Minchiotti et al., Biochim Biophys Acta 1987:916:411-418), D550G (Takahashi et al., PNAS 1987:84:4413-7), and D550A (Carlson et al., PNAS 1992:89:8225-9). The numbering of these natural variants is based on mature HSA (SEQ ID NO: 36). Albumin variants for genetic fusion are also commercially available (e.g., Albufuse® Flex and Recombumin® Flex, Novozymes).

One or more positions of albumin, or a variant or fragment thereof, can be altered to provide reactive surface residues for, e.g., conjugation with IL-2 or a mutant thereof. Exemplary positions in HSA (SEQ ID NO: 35 or 36) that can be altered to provide conjugation competent cysteine residues include, but are not limited to, those disclosed in WO2010/092135, such as, D1C, A2C, T79C, E82C, E86C, D121C, D129C, S270C, A364C, A504C, E505C, D549C, D562C, A578C, A579C, A581C, L585C, and L595C (the numbering of these amino acid residues is based on mature HSA (SEQ ID NO: 36). Alternatively a cysteine residue may be added to the N or C terminus of albumin. Methods suitable for producing conjugation competent albumin, or a variant or peptide thereof, as well as covalently linking albumin, or a variant or fragment thereof, with a conjugation partner or partners (e.g., IL-2 or a mutant thereof) are routine in the art and disclosed in, e.g., WO2010/092135 and WO 2009/019314. In one embodiment, the conjugates may conveniently be linked via a free thio group present on the surface of HSA (amino acid residue 34 of mature HSA (SEQ ID NO: 36)) using art-recognized methods.

In addition to the albumin or variants thereof described supra, fragments of albumin, or fragments of variants thereof, are suitable for use as a PK moiety. Exemplary albumin fragments are disclosed in WO 2011/124718. A fragment of albumin (e.g., a fragment of HSA) will typically be at least 20 amino acids in length, such as at least 40 amino acids, at least 60 amino acids, at least 80 amino acids, at least 100 amino acids, at least 150 amino acids, at least 200 amino acids, at least 300 amino acids, at least 400 amino acids, or at least 500 amino acids in length, and will increase the serum half-life of IL-2 or a mutant thereof, to which it is fused to relative to the non-fused IL-2 or IL-2 mutant. In some embodiments, a fragment may comprise at least one whole sub-domain of albumin. Domains of HSA have been expressed as recombinant proteins (Dockal et al., TBC 1999; 274:29303-10), where domain I was defined as consisting of amino acids 1-197, domain II was defined as consisting of amino acids 189-385, and domain III was defined as consisting of amino acids 381-585 of HSA (SEQ ID NO: 36). A fragment may comprise or consist of at least 50, 60, 70, 75, 80, 85, 90, 95, 96, 97, 98, or 99% of an albumin or of a domain of an albumin, or a variant or fragment thereof. Additionally, single or multiple heterologous fusions comprising any of the above; or single or multiple heterologous fusions to albumin, or a variant or fragment of any of these may be used. Such fusions include albumin N-terminal fusions, albumin C-terminal fusions and co-N-terminal and C-terminal albumin fusions as exemplified by WO 01/79271.

Methods of fusing serum albumin to proteins are disclosed in, e.g., US2010/0144599, US2007/0048282, and US2011/0020345, which are herein incorporated by reference in their entirety.

(v) Other Extended-PK Groups

In some embodiments, the extended-PK group is transferrin, as disclosed in U.S. Pat. No. 7,176,278 and U.S. Pat. No. 8,158,579, which are herein incorporated by reference in their entirety.

In some embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety.

In some embodiments, the extended-PK group is a serum immunoglobulin binding protein such as those disclosed in US2007/0178082, which is herein incorporated by reference in its entirety.

In some embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3(HSA), i.e., a Fn3 protein that binds to human serum albumin.

(vi) Linkers

In some embodiments, the extended-PK group is optionally fused to IL-2 via a linker. Linkers suitable for fusing the extended-PK group to IL-2 are well known in the art, and are disclosed in, e.g., US2010/0210511 US2010/0179094, and US2012/0094909, which are herein incorporated by reference in its entirety. Exemplary linkers include gly-ser polypeptide linkers, glycine-proline polypeptide linkers, and proline-alanine polypeptide linkers. In a preferred embodiment, the linker is a gly-ser polypeptide linker, i.e., a peptide that consists of glycine and serine residues.

Exemplary gly-ser polypeptide linkers comprise the amino acid sequence Ser(Gly₄Ser)n. In one embodiment, n=1. In one embodiment, n=2. In another embodiment, n=3, i.e., Ser(Gly₄Ser)₃. In another embodiment, n=4, i.e., Ser(Gly₄Ser)₄. In another embodiment, n=5. In yet another embodiment, n=6. In another embodiment, n=7. In yet another embodiment, n=8. In another embodiment, n=9. In yet another embodiment, n=10. Another exemplary gly-ser polypeptide linker comprises the amino acid sequence Ser(Gly₄Ser)n. In one embodiment, n=1. In one embodiment, n=2. In a preferred embodiment, n=3. In another embodiment, n=4. In another embodiment, n=5. In yet another embodiment, n=6. Another exemplary gly-ser polypeptide linker comprises (Gly₄Ser)n. In one embodiment, n=1. In one embodiment, n=2. In a preferred embodiment, n=3. In another embodiment, n=4. In another embodiment, n=5. In yet another embodiment, n=6. Another exemplary gly-ser polypeptide linker comprises (Gly₃Ser)n. In one embodiment, n=1. In one embodiment, n=2. In a preferred embodiment, n=3. In another embodiment, n=4. In another embodiment, n=5. In yet another embodiment, n=6.

### Adoptive Cell Therapy

Adoptive cell therapy (ACT) is a treatment method where cells are removed from a donor, cultured and/or manipulated in vitro, and administered to a patient for the treatment of a disease. To date, clinical results of ACT monotherapy have been marginal, due in part to the difficulty in promoting the long term proliferation and survival of the transferred cells. In accordance with the present invention, extended-PK IL-2 is administered to a subject receiving ACT. Administration of extended-PK IL-2 in combination with ACT promotes the persistence and proliferation of transferred cells, relative to patients receiving ACT as a monotherapy, while minimizing the adverse side-effects associated with co-administration of free IL-2.

The instant invention relates broadly to the discovery that the outcome of ACT can be improved by administration of extended-PK IL-2 to cancer subjects receiving ACT, optionally in conjunction with a therapeutic antibody. A variety of ACT approaches have been described in the art for the treatment of several conditions, including cancer. By promoting the persistence and proliferation of transferred cells, extended-PK IL-2 is beneficial when administered in conjunction with all types of cancer-directed ACT. Exemplary strategies for ACT employ, for example, tumor infiltrating lymphocytes (TIL), antigen-expanded CD8+ and/or CD4+ T cells, T cells genetically modified to express a T cell receptor (TCR) that specifically recognizes a tumor antigen, and T cells genetically modified to express a chimeric antigen receptor (CAR). These strategies have been well-documented in the art, and a brief description of each of these approaches is set forth below. This brief description is not intended to be limiting. These and other approaches for ACT are well-documented in the scientific literature, and can be used in combination with extended-PK IL-2 (and optionally a therapeutic antibody) in accordance with the instant invention.

(A) Tumor Infiltrating Lymphocyes (TIL)

One ACT strategy involves the transplantation of autologous TIL expanded ex vivo from tumor fragments or single cell enzymatic digests of tumor metastases. T cell infiltrates in tumors are polyclonal in nature and collectively recognize multiple tumor antigens. This approach was first used successfully in 1988 (Rosenberg et al., N. Engl. J. Med. (1988) 319:1676-1680), and subsequent developments have improved the overall response rate of autologous TIL therapy.

In an exemplary TIL ACT protocol, tumors are resected from patients and are cut into small (3-5 mm²) fragments under sterile conditions. The fragments are placed into culture plates or flasks with growth medium and are treated with high-dose IL-2. This initial TIL expansion-phase (also known as the “Pre-REP” phase) typically lasts 3-5 weeks, during which time about 5×10⁷ or more TILs are produced. The resulting TILs are then further expanded (e.g., following a rapid expansion protocol (REP)) to produce TILs suitable for infusion into a subject. The pre-REP TILs can be cryopreserved for later expansion, or they may be expanded immediately. Pre-REP TILs can also be screened to identify cultures with high anti-tumor reactivity prior to expansion. A typical REP involves activating TILs using a T-cell stimulating antibody, e.g., an anti-CD3 mAb, in the presence of irradiated PBMC feeder cells. The feeder cells can be obtained from the patient or from healthy donor subjects. IL-2 is often added to the REP culture at concentrations of about 6,000 U/mL to promote rapid TIL cell division. Expansion of TILs in this manner can take about 2 weeks or longer, and results in a pool of about 10-150 billion TILs. The expanded cells are washed and pooled, and are suitable for infusion into a patient. Patients typically receive 1 or 2 infusions (separated by 1-2 weeks) of 10⁹->10¹¹ cells. Patients have been administered high-dose IL-2 therapy (e.g., 7.2×10⁵ IU/kg every 8 hours for 2-3 days) to help support the TIL cells after infusion (Rosenberg et al., Nat. Rev. Cancer (2008) 8:299-308). Using extended-PK IL-2 in place of free IL-2 in accordance with the instant invention further promotes the persistence, proliferation, and survival of transferred TIL cells, and improves tumor regression, while avoiding the negative effects of IL-2 therapy.

Before infusion, a patient can optionally be lymphodepleted using cyclophosphamide (Cy) and fludaribine (Flu) (see, e.g., Dudley et al., Science (2003) 298:850-854). In addition, in order to prevent the re-emergence of endogenous regulatory T cells (Tregs), total body irradiation (TBI) has been used with lymphodepletion (see, e.g., Dudley et al., J. Clin. Oncol. (2008) 26(32):5233-5239).

(B) Antigen-Expanded CD8+ and/or CD4+ T Cells

Autologous peripheral blood mononuclear cells (PBMC) can be stimulated in vitro with antigen to generate tumor antigen-specific or polyclonal CD8+ and/or CD4+ T cell clones that can be used for ACT (see, e.g., Mackensen et al., J. Clin. Oncol. (2006) 24(31):5060-5069; Mitchell et al., J. Clin. Oncol. (2002) 20(4):1075-1086; Yee et al., Proc. Natl. Aad. Sci. USA (2002) 99(25):16168-16173; Hunder et al., N. Engl. J. Med. (2008) 358(25):2698-2703; Verdegaal et al., Cancer Immunol. Immunother. (2001) 60(7):953-963). In order to avoid the time-consuming and labor-intensive process of expanding tumor-specific T cells from naïve PBMC populations, a new approach has been recently described in which antigen-specific T cells for ACT are generated using multiple stimulation of autologous PBMC using artificial antigen-presenting cells (aAPC) expressing HLA-A0201, costimulatory molecules, and membrane-bound cytokines (see, e.g., Suhoski et al., Mol. Ther. (2007) 15(5):981-988; Butler et al., Sci. Transl. Med. (2011) 3(80):80ra34).

In one embodiment, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

In one embodiment, the cell population is enriched for CD8+ T cells. A T cell culture may be depleted of CD4+ cells and enriched for CD8+ cells using, for example, a CD8 microbead separation (e.g., using a Clini-MACSP^{plus} CD8 microbead system (Miltenyi Biotec). Enriching for CD8+ T cells may improve the outcome of ACT by removing CD4+ T regulatory cells.

Administering extended-PK IL-2, optionally in combination with a therapeutic antibody, to subjects receiving an ACT regimen involving infusion of CD8+ and/or CD4+ T cells obtained from stimulation of PBMCs promotes the persistence of the transferred cells, stimulates the persistence, proliferation and survival of transferred cells, and improves tumor regression, while avoiding the negative effects of IL-2 therapy.

(C) T Cells Genetically Modified to Express a T Cell Receptor (TCR) that Specifically Recognizes a Tumor Antigen

In some instances, it is not possible to obtain TILs with high avidity for tumor antigens in the quantity necessary for ACT. Accordingly, it may be desirable to genetically modify lymphocytes to obtain a cell population that specifically recognizes an antigen of interest prior to infusion into a subject. Genes encoding TCRs can be isolated from T cells that specifically recognize cancer antigens with high avidity. T lymphocytes isolated from peripheral blood can be transduced with a retrovirus that contains genes encoding TCRs possessing the desired specificity. This method permits the rapid production to a large number of tumor-antigen-specific T cells for ACT.

T cells may be transduced to express a T cell receptor (TCR) having antigenic specificity for a cancer antigen using transduction techniques described in Heemskerk et al. Hum Gene Ther. 19:496-510 (2008) and Johnson et al. Blood 114:535-46 (2009). ACT using T cells genetically modified to express a TCR recognizing an antigen of interest can be performed in accordance with the clinical trial protocol published by Morgan et al., Science (2006) 314(5796):126-129. Administering extended-PK IL-2, optionally in combination with a therapeutic antibody, to subjects receiving an ACT regimen involving administration of T cells that have been genetically engineered to express a TCR (or modified TCR) recognizing a tumor antigen promotes the persistence of the transferred cells, stimulates the persistence, proliferation and survival of transferred cells, and improves tumor regression, while avoiding the negative effects of IL-2 therapy.

In some embodiments, T cells may be transduced with a modified TCR. Modifications may be made, for example, to enhance the ability to recognize target cells when expressed by CD4+ T cells and/or CD8+ T cells. Modified TCRs and methods of making modified TCRs are described in, for example, US Patent Publication Nos. US 2010/0297093A1, and US 2012/0015888A1, and U.S. Pat. No. 8,088,379, the contents of which are incorporated herein by reference in their entirety.

In a treatment regimen that involves administration of a therapeutic antibody with extended-PK IL-2 and genetically engineered T cells expressing a TCR that specifically recognizes a protein of interest (e.g., a tumor antigen), the antibody may recognize the same protein as the TCR. In another embodiment, the antibody recognizes another tumor antigen expressed on cells of the subject's cancer.

(D) T Cells Genetically Modified to Express a Chimeric Antigen Receptor (CAR)

Genetic engineering of T cells to express a TCR having a desired specificity as described above is a very promising approach for ACT. Notwithstanding, there is the potential for mispairing of the engineered TCR alpha and beta chains with endogenous TCR chains. In addition, the success of ACT using cells expressing engineered TCR depends on expression of the specific MHC molecule recognized by the TCR in the targeted cancer cells. To avoid these potential complications, T cells may alternatively be engineered to express chimeric antigen receptors (CARs).

In their simplest form, CARs contain an antigen binding domain coupled with the transmembrane domain and the signaling domain from the cytoplasmic tail of the CD3 ζ chain. There is some evidence that the CD3 ζ chain is insufficient to fully activate transduced T cells. Accordingly, CARs preferably contain an antigen binding domain, a costimulatory domain, and a CD3 ζ signaling domain. Using a costimulatory domain in combination with the CD3 ζ signaling domain mimics the two-signal model of T cell activation.

The CAR antigen binding domain can be an antibody or antibody fragment, such as, for example, a Fab or an scFv. Non-limiting examples of anti-cancer antibodies include the following, without limitation:

trastuzumab (HERCEPTIN™ by Genentech, South San Francisco, Calif.), which is used to treat HER-2/neu positive breast cancer or metastatic breast cancer;

bevacizumab (AVASTIN™ by Genentech), which is used to treat colorectal cancer, metastatic colorectal cancer, breast cancer, metastatic breast cancer, non-small cell lung cancer, or renal cell carcinoma;

rituximab (RITUXAN™ by Genentech), which is used to treat non-Hodgkin's lymphoma or chronic lymphocytic leukemia;

pertuzumab (OMNITARG™ by Genentech), which is used to treat breast cancer, prostate cancer, non-small cell lung cancer, or ovarian cancer;

cetuximab (ERBITUX™ by ImClone Systems Incorporated, New York, N.Y.), which can be used to treat colorectal cancer, metastatic colorectal cancer, lung cancer, head and neck cancer, colon cancer, breast cancer, prostate cancer, gastric cancer, ovarian cancer, brain cancer, pancreatic cancer, esophageal cancer, renal cell cancer, prostate cancer, cervical cancer, or bladder cancer;

IMC-1C11 (ImClone Systems Incorporated), which is used to treat colorectal cancer, head and neck cancer, as well as other potential cancer targets;

tositumomab and tositumomab and iodine I¹³¹ (BEXXAR™ by Corixa Corporation, Seattle, Wash.), which is used to treat non-Hodgkin's lymphoma, which can be CD20 positive, follicular, non-Hodgkin's lymphoma, with and without transformation, whose disease is refractory to Rituximab and has relapsed following chemotherapy;

In¹¹¹ ibirtumomab tiuxetan; Y⁹⁰ ibirtumomab tiuxetan; In¹¹¹ ibirtumomab tiuxetan and Y⁹⁰ ibirtumomab tiuxetan (ZEVALIN™ by Biogen Idec, Cambridge, Mass.), which is used to treat lymphoma or non-Hodgkin's lymphoma, which can include relapsed follicular lymphoma; relapsed or refractory, low grade or follicular non-Hodgkin's lymphoma; or transformed B-cell non-Hodgkin's lymphoma;

EMD 7200 (EMD Pharmaceuticals, Durham, N.C.), which is used for treating for treating non-small cell lung cancer or cervical cancer;

SGN-30 (a genetically engineered monoclonal antibody targeted to CD30 antigen by Seattle Genetics, Bothell, Wash.), which is used for treating Hodgkin's lymphoma or non-Hodgkin's lymphoma;

SGN-15 (a genetically engineered monoclonal antibody targeted to a Lewisγ-related antigen that is conjugated to doxorubicin by Seattle Genetics), which is used for treating non-small cell lung cancer;

SGN-33 (a humanized antibody targeted to CD33 antigen by Seattle Genetics), which is used for treating acute myeloid leukemia (AML) and myelodysplastic syndromes (MDS);

SGN-40 (a humanized monoclonal antibody targeted to CD40 antigen by Seattle Genetics), which is used for treating multiple myeloma or non-Hodgkin's lymphoma;

SGN-35 (a genetically engineered monoclonal antibody targeted to a CD30 antigen that is conjugated to auristatin E by Seattle Genetics), which is used for treating non-Hodgkin's lymphoma;

SGN-70 (a humanized antibody targeted to CD70 antigen by Seattle Genetics), that is used for treating renal cancer and nasopharyngeal carcinoma;

SGN-75 (a conjugate comprised of the SGN70 antibody and an Auristatin derivative by Seattle Genetics); and

SGN-17/19 (a fusion protein containing antibody and enzyme conjugated to melphalan prodrug by Seattle Genetics), which is used for treating melanoma or metastatic melanoma.

It should be understood that the therapeutic antibodies to be used in the methods of the present invention are not limited to those described supra. For example, the following approved therapeutic antibodies can also be used in the methods of the invention: brentuximab vedotin (ADCETRIS™) for anaplastic large cell lymphoma and Hodgkin lymphoma, ipilimumab (MDX-101; YERVOY™) for melanoma, ofatumumab (ARZERRA™) for chromic lymphocytic leukemia, panitumumab (VECTIBIX™) for colorectal cancer, alemtuzumab (CAMPATH™) for chronic lymphocytic leukemia, ofatumumab (ARZERRA™) for chronic lymphocytic leukemia, gemtuzumab ozogamicin (MYLOTARG™) for acute myelogenous leukemia.

Antibodies for use in the present invention can also target molecules expressed by immune cells, such as, but not limited to, tremelimumab (CP-675,206) and ipilimumab (MDX-010) which targets CTLA4 and has the effect of tumor rejection, protection from rechallenge, and enhanced tumor-specific T cell responses; OX86 which targets OX40 and increases antigen-specific CD8+ T cells at tumor sites and enhances tumor rejection; CT-011 which targets PD 1 and has the effect of maintaining and expanding tumor specific memory T cells and activates NK cells; BMS-663513 which targets CD137 and causes regression of established tumors, as well as the expansion and maintenance of CD8+ T cells, and daclizumab (ZENAPAX™) which targets CD25 and causes transient depletion of CD4+CD25+FOXP3+ Tregs and enhances tumor regression and increases the number of effector T cells. A more detailed discussion of these antibodies can be found in, e.g., Weiner et al., Nature Rev. Immunol (2010); 10:317-27.

Other therapeutic antibodies can be identified that target tumor antigens (e.g., tumor antigens associated with different types of cancers, such as carcinomas, sarcomas, myelomas, leukemias, lymphomas, and combinations thereof). For example, the following tumor antigens can be targeted by therapeutic antibodies that may be administered in combination with ACT.

The tumor antigen may be an epithelial cancer antigen, (e.g., breast, gastrointestinal, lung), a prostate specific cancer antigen (PSA) or prostate specific membrane antigen (PSMA), a bladder cancer antigen, a lung (e.g., small cell lung) cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen. In another embodiment, the tumor antigen is a lymphoma antigen (e.g., non-Hodgkin's lymphoma or Hodgkin's lymphoma), a B-cell lymphoma cancer antigen, a leukemia antigen, a myeloma (i.e., multiple myeloma or plasma cell myeloma) antigen, an acute lymphoblastic leukemia antigen, a chronic myeloid leukemia antigen, or an acute myelogenous leukemia antigen. It should be understood that the described tumor antigens are only exemplary and that any tumor antigen can be targeted in the present invention.

In another embodiment, the tumor antigen is a mucin-1 protein or peptide (MUC-1) that is found on most or all human adenocarcinomas: pancreas, colon, breast, ovarian, lung, prostate, head and neck, including multiple myelomas and some B cell lymphomas. Patients with inflammatory bowel disease, either Crohn's disease or ulcerative colitis, are at an increased risk for developing colorectal carcinoma. MUC-1 is a type I transmembrane glycoprotein. The major extracellular portion of MUC-1 has a large number of tandem repeats consisting of 20 amino acids which comprise immunogenic epitopes. In some cancers it is exposed in an unglycosylated form that is recognized by the immune system (Gendler et al., J Biol Chem 1990; 265:15286-15293). In another embodiment, the tumor antigen is a mutated B-Raf antigen, which is associated with melanoma and colon cancer. The vast majority of these mutations represent a single nucleotide change of T-A at nucleotide 1796 resulting in a valine to glutamic acid change at residue 599 within the activation segment of B-Raf. Raf proteins are also indirectly associated with cancer as effectors of activated Ras proteins, oncogenic forms of which are present in approximately one-third of all human cancers. Normal non-mutated B-Raf is involved in cell signaling, relaying signals from the cell membrane to the nucleus. The protein is usually only active when needed to relay signals. In contrast, mutant B-Raf has been reported to be constantly active, disrupting the signaling relay (Mercer and Pritchard, Biochim Biophys Acta (2003) 1653(1):25-40; Sharkey et al., Cancer Res. (2004) 64(5):1595-1599).

In one embodiment, the tumor antigen is a human epidermal growth factor receptor-2 (HER-2/neu) antigen. Cancers that have cells that overexpress HER-2/neu are referred to as HER-2/neu⁺ cancers. Exemplary HER-2/neu⁺ cancers include prostate cancer, lung cancer, breast cancer, ovarian cancer, pancreatic cancer, skin cancer, liver cancer (e.g., hepatocellular adenocarcinoma), intestinal cancer, and bladder cancer.

HER-2/neu has an extracellular binding domain (ECD) of approximately 645 aa, with 40% homology to epidermal growth factor receptor (EGFR), a highly hydrophobic transmembrane anchor domain (TMD), and a carboxyterminal intracellular domain (ICD) of approximately 580 aa with 80% homology to EGFR. The nucleotide sequence of HER-2/neu is available at GENBANK™. Accession Nos. AH002823 (human HER-2 gene, promoter region and exon 1); M16792 (human HER-2 gene, exon 4): M16791 (human HER-2 gene, exon 3); M16790 (human HER-2 gene, exon 2); and M16789 (human HER-2 gene, promoter region and exon 1). The amino acid sequence for the HER-2/neu protein is available at GENBANK™. Accession No. AAA58637. Based on these sequences, one skilled in the art could develop HER-2/neu antigens using known assays to find appropriate epitopes that generate an effective immune response. Exemplary HER-2/neu antigens include p369-377 (a HER-2/neu derived HLA-A2 peptide); dHER2 (Corixa Corporation); li-Key MHC class II epitope hybrid (Generex Biotechnology Corporation); peptide P4 (amino acids 378-398); peptide P7 (amino acids 610-623); mixture of peptides P6 (amino acids 544-560) and P7; mixture of peptides P4, P6 and P7; HER2 [9₇₅₄]; and the like.

In one embodiment, the tumor antigen is an epidermal growth factor receptor (EGFR) antigen. The EGFR antigen can be an EGFR variant 1 antigen, an EGFR variant 2 antigen, an EGFR variant 3 antigen and/or an EGFR variant 4 antigen. Cancers with cells that overexpress EGFR are referred to as EGFR⁺ cancers. Exemplary EGFR⁺ cancers include lung cancer, head and neck cancer, colon cancer, colorectal cancer, breast cancer, prostate cancer, gastric cancer, ovarian cancer, brain cancer and bladder cancer.

In one embodiment, the tumor antigen is a vascular endothelial growth factor receptor (VEGFR) antigen. VEGFR is considered to be a regulator of cancer-induced angiogenesis. Cancers with cells that overexpress VEGFR are called VEGFR⁺ cancers. Exemplary VEGFR⁺ cancers include breast cancer, lung cancer, small cell lung cancer, colon cancer, colorectal cancer, renal cancer, leukemia, and lymphocytic leukemia.

In one embodiment the tumor antigen is prostate-specific antigen (PSA) and/or prostate-specific membrane antigen (PSMA) that are prevalently expressed in androgen-independent prostate cancers.

In another embodiment, the tumor antigen is Gp-100 Glycoprotein 100 (gp 100) is a tumor-specific antigen associated with melanoma.

In one embodiment, the tumor antigen is a carcinoembryonic (CEA) antigen. Cancers with cells that overexpress CEA are referred to as CEA⁺ cancers. Exemplary CEA⁺ cancers include colorectal cancer, gastric cancer and pancreatic cancer. Exemplary CEA antigens include CAP-1 (i.e., CEA aa 571-579), CAP1-6D, CAP-2 (i.e., CEA aa 555-579), CAP-3 (i.e., CEA aa 87-89), CAP-4 (CEA aa 1-11), CAP-5 (i.e., CEA aa 345-354), CAP-6 (i.e., CEA aa 19-28) and CAP-7.

In one embodiment, the tumor antigen is carbohydrate antigen 10.9 (CA 19.9). CA 19.9 is an oligosaccharide related to the Lewis A blood group substance and is associated with colorectal cancers.

In another embodiment, the tumor antigen is a melanoma cancer antigen. Melanoma cancer antigens are useful for treating melanoma. Exemplary melanoma cancer antigens include MART-1 (e.g., MART-1 26-35 peptide, MART-1 27-35 peptide); MART-1/Melan A; pMel17; pMel17/gp100; gp100 (e.g., gp 100 peptide 280-288, gp 100 peptide 154-162, gp 100 peptide 457-467); TRP-1; TRP-2; NY-ESO-1; p16; beta-catenin; mum-1; and the like.

In one embodiment, the tumor antigen is a mutant or wild type ras peptide. The mutant ras peptide can be a mutant K-ras peptide, a mutant N-ras peptide and/or a mutant H-ras peptide. Mutations in the ras protein typically occur at positions 12 (e.g., arginine or valine substituted for glycine), 13 (e.g., asparagine for glycine), 61 (e.g., glutamine to leucine) and/or 59. Mutant ras peptides can be useful as lung cancer antigens, gastrointestinal cancer antigens, hepatoma antigens, myeloid cancer antigens (e.g., acute leukemia, myelodysplasia), skin cancer antigens (e.g., melanoma, basal cell, squamous cell), bladder cancer antigens, colon cancer antigens, colorectal cancer antigens, and renal cell cancer antigens.

In another embodiment of the invention, the tumor antigen is a mutant and/or wildtype p53 peptide. The p53 peptide can be used as colon cancer antigens, lung cancer antigens, breast cancer antigens, hepatocellular carcinoma cancer antigens, lymphoma cancer antigens, prostate cancer antigens, thyroid cancer antigens, bladder cancer antigens, pancreatic cancer antigens and ovarian cancer antigens.

In a preferred embodiment, the antigen binding domain recognizes a tumor antigen, as described, e.g., in WO2008/131052. Tumor antigens are proteins that are produced by tumor cells that elicit an immune response, particularly T-cell mediated immune responses. The selection of the antigen binding moiety will depend on the particular type of cancer to be treated. Tumor antigens are well known in the art and include, for example, a glioma-associated antigen, carcinoembryonic antigen (CEA), 13-human chorionic gonadotropin, alphafetoprotein (AFP), lectin-reactive AFP, thyroglobulm, RAGE-1, MN-CA IX, human telomerase reverse transcriptase, RU1, RU2 (AS), intestinal carboxyi esterase, mut hsp70-2, M-CSF, prostase, prostate-specific antigen (PSA), PAP, NY-ESO-1, LAGE-la, p53, tyrosinase, prostein, PSMA, ras, Her2/neu, TRP-1, TRP-2, TAG-72, KSA, CA-125, PSA, BRCI, BRC-II, bcr-abl, pax3-fkhr, ews-fli-1, survivin and telomerase, prostate-carcinoma tumor antigen-1 (PCTA-1), MAGE, GAGE, GP-100, MUC-1, MUC-2, ELF2M, neutrophil elastase, ephrinB2, CD22, insulin growth factor (IGF)-I, IGF-II, IGF-I receptor, and mesothelin,

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD 19, CD20 and CD37 are other candidates for target antigens in &-cell lymphoma, Some of these antigens (CEA, HER-2, CD19, CD20, idiotype) have been used as targets for passive immunotherapy with monoclonal antibodies with limited success.

The tumor antigen may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-1), Pmel 17, tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations such as BCR-E2A-PRL, H4-RET, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p 180erbB-3, c-met, nm-23H1 PSA, TAG-72, CA 19-9, CA 72-4, CAM 17.1, NuMa, K-ras, beta-Catenin, CDK4, Mum-1, p 15, p 16, 43-9F, 5T4(791Tgp72₎ alpha-fetoprotem, beta-HCG, BCA225, BTAA, CA 125, CA 15-3\CA\27.29\BCAA, CA 195, CA 242, CA-50, CAM43, CD68\I, CO-029, FGF-5, G250, Ga733VEpCAM, HTgp-175, M344, MA-50, MG7-Ag, MOV 18, NB/70K, NY-CO-1, RCAS 1, SDCCAG16, TA-90\Mac-2 binding protein, Acyclophilin C-associated protein, TAAL6, TAG72, TLP, and TPS.

In a preferred embodiment, the antigen binding moiety portion of the CAR targets antigen that includes but is not limited to CD19, CD20, CD22, ROR 1, Mesothelin, CD33/IL3Ra, c-Met, PSMA, Glycolipid F77, EGFRvIII, GD-2, NY-ESO-1 TCR, MACE A3 TCR, and the like.

Other relevant cancer antigens include those disclosed in Cheever et al., Clin Cancer Res 2009; 15:5323-37, the contents of which are herein incorporated by reference.

The foregoing mention of exemplary tumor antigens targeted by therapeutic antibodies is not intended to be limiting. Identifying therapeutic antibodies that recognize a tumor antigen of interest is within the ability of a person of ordinary skill

The antigen binding domain is separated from the CD3 ζ signaling domain and the costimulatory domain by a transmembrane domain. The transmembrane domain may be derived from any transmembrane protein. In one embodiment, a transmembrane domain that is naturally associated with one of the domains in the CAR is used. In another embodiment, an exogenous or synthetic transmembrane domain is used. In some embodiments, the transmembrane domain can be selected or modified by amino acid substitution to minimize interactions with other membrane proteins.

Between the extracellular domain and the transmembrane domain of the CAR, or between the cytoplasmic domain and the transmembrane domain of the CAR, a spacer may optionally be incorporated. The spacer may be any oligo- or polypeptide that functions to link the transmembrane domain to either the extracellular domain or the cytoplasmic domain. A spacer may comprise up to 300 amino acids, preferably 10 to 100 amino acids, and more preferably 25 to 50 amino acids.

The intracellular domain of a CAR is responsible for activation of at least one of the normal effector functions of the immune cell in which the CAR is expressed. Effector functions may include, for example, cytolytic activity or helper activity, such as the secretion of cytokines. Thus the intracellular signaling domain of a molecule refers to the portion of a protein which transduces the effector function signal and directs the cell to perform a specialized function. While the entire intracellular signaling domain can be used, in many cases a portion of the intracellular domain may be used, so long as the selected portion transduces the effector function signal. The cytoplasmic domain of a CAR can include the CD3 ζ signaling domain on its own, or in combination with a costimulatory domain. The costimulatory domain contains the intracellular domain of a costimulatory molecule. Costimulatory molecules are cell surface molecules that promote an efficient response of lymphocytes to antigen. In some embodiments, the costimulatory domain contains an intracellular domain of a costimulatory molecule such as 4-1BB, CD27, CD28, OX40, CD30, CD40, PD-1, ICOS, lymphocyte function-associated antigen-1 (LFA-1), CD2, CD7, LIGHT, NKG2C, B7-H3, a CD83 ligand, or combinations thereof. In an exemplary embodiment, the costimulatory molecule is the intracellular domain of 4-1BB or CD28.

Additional detail regarding the construction and use of CARs can be found in International Publication No. WO 2012/079000A1, the contents of which are incorporated by reference herein in its entirety. In certain embodiments, a T cell is engineered to express a CAR, wherein the CAR comprises an antigen binding domain derived from a bispecific antibody, as disclosed in WO2014011988, the contents of which are incorporated by reference herein in their entirety. In certain embodiments, a plurality of types of CARs participate in trans-signaling to induce T cell activation (e.g., a first CAR having a first signaling module and a second CAR having a distinct second signaling module, wherein activation of the T cell depends on the binding of the first CAR to its target and the binding of the second CAR to its target), as disclosed in US20140099309, the contents of which are incorporated by reference herein in their entirety.

The following CAR constructs are currently being pursued in clinical trials for various oncology indications: anti-GD-2 CAR (in combination with iCaspase suicide safety switch) for neuroblastoma (NCT01822652) and non-neuroblastoma (NCT02107963) GD2+ solid tumors; anti-GD2 CAR for refractory or metastatic GD2-positive sarcoma (NCT01953900); anti-GD2 CAR for relapsed/refractory neuroblastoma (NCT01460901); anti-CD19 CAR for patients with recurrent or persistent B-cell malignancies after allogeneic stem cell transplantation (NCT01087294), anti-CD19 CAR for pediatric patients with relapsed CD19+ acute lymphoblastic leukemia; anti-CD19 CAR for relapsed and refractory aggressive CD19+ B cell non-Hodgkin lymphoma (NCT01840566); anti-CD19 CAR for relapsed and refractory B cell non-Hodgkin lymphoma (NCT02134262); anti-CD19 CAR for relapsed or refractory CLL or SLL (NCT01747486); anti-CD19 CAR for mantle cell lymphoma (NCT02081937); anti-CD19 CAR for post-allo HSCT (NCT02050347); anti-CD19 CAR for relapsed/refractory CD19+ leukemia (NCT02028455); anti-CD19 CAR for chronic lymphocytic leukemia, small lymphocytic lymphoma, mantle cell lymphoma, follicular lymphoma, and large cell lymphoma (NCT00924326); anti-CD19 CAR for B cell malignancies after allogeneic transplant (NCT01475058); anti-CD19 CAR for relapsed or refractory chronic lymphocytic leukemia, non-Hodgkin lymphoma, or acute lymphoblastic leukemia (NCT01865617); anti-CD19 CAR for advanced B cell NHL and CLL (NCT01853631); anti-CD 19 CAR for high-risk, intermediate-grade, B cell non-Hodgkin lymphoma after peripheral blood stem cell transplant (NCT01318317); anti-CD19 CAR for children and young adults with B cell leukemia or lymphoma (NCT01593696); anti-CD19 CAR for pediatric and young adult patients with relapsed B cell acute lymphoblastic leukemia (NCT01860937); anti-CD19 CAR for refractory B cell malignancy (NCT02132624); anti-Her2 CAR for advanced sarcoma (NCT00902044); anti-CD 19 CAR for CD19 positive residual or relapsed acute lymphoblastic leukemia after allogeneic hematopoietic progenitor cell transplantation (NCT01430390); anti-CD19 CAR for chemotherapy resistant or refractory CD19+ leukemia and lymphoma (NCT01626495); anti-CD19 CAR attached to TCRz and 4-signaling domains for chemotherapy relapsed or refractory CD19+ lymphomas (NCT02030834); anti-CD19 CAR attached to TCR and 4-1BB signaling domains for patients with chemotherapy resistant or refractory ALL (NCT02030847); anti-CD19 CAR for relapsed and/or chemotherapy refractory B cell malignancy (NCT01864889); anti-CD19:4-1BB:CD3 ζ CAR for B cell leukemia or lymphoma resistant or refractory to chemotherapy; anti-CD19 CAR for CD19+ malignancy (NCT01493453); anti-CD19 CAR for precursor B-ALL (NCT01044069); anti-Her2 CAR for glioblastoma multiforme (NCT01109095); anti-Her2 and TGF-beta for Her2 positive malignancy (NCT00889954); anti-Her2 CAR for chemotherapy refractory Her2+ advanced solid tumors (NCT01935843); anti-LewisY CAR for myeloma, acute myeloid leukemia, or myelodyslpastic syndrome (NCT01716364); anti-kappa light chain-CD28 CAR for chronic lymphocytic leukemia, B cell lymphoma, or multiple myeloma (NCT00881920); anti-CD30 CAR for Hodgkin's lymphoma and non-Hodgkin's lymphoma (NCT01316146); anti-EGFR CAR for EGFR+ advanced solid tumors (NCT01869166); anti-EGFR-III CAR for malignant gliomas (NCT01454596); anti-CD33 CAR for relapsed and/or chemotherapy refractory CD33 positive acute myeloid leukemia; anti-CD138 CAR for chemotherapy refractory multiple myeloma (NCT01886976); anti-FAP CAR for FAP-positive malignant pleural mesothelioma (NCT01722149); anti-CEA MFEz CAR for cancer (NCT01212887); and anti-CEA CAR for adenocarcinoma (NCT01723306).

(E) Other Genetic Modifications to T Cells

T cells can be further engineered express proteins that enhance anti-tumor activity, for example, as described in Kershaw et al. Nature Reviews Cancer 2013; 13:525-41. Exemplary proteins include, but are not limited to, cytokines (IL-2, IL-12), anti-apoptotic molecules (BCL-2, BCL-X), and chemokines (CXCR2, CCR4, CCR2B).

(F) Nonmyeloablative Lymphodepleting Chemotherapy

In one embodiment of any of the foregoing ACT approaches, a subject is administered nonmyeloablative lymphodepleting chemotherapy prior to the transfer of autologous cells. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine. A preferred route of administering cyclophosphamide and fludarabine is intravenously. Likewise, any suitable dose of cyclophosphamide and fludarabine can be administered. In one embodiment, around 60 mg/kg of cyclophosphamide is administered for two days, after which around 25 mg/m² fludarabine is administered for five days.

(G) Sources of T cells

Prior to expansion and genetic modification of T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments, any number of T cell lines available in the art may be used. In certain embodiments of the present invention, T cells can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. The cells may be washed with phosphate buffered saline (PBS), or with a wash solution that lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 ceil processor, the Baxter CytoMate, or the Haemonetics Cell Saver 5) according to the manufacturer's instructions. After washing, the cells may be resuspended in a variety of biocompatible buffers, such as, for example, Ca³⁺-free, Mg²⁺-free PBS, PlasmaLyte A, or other saline solution with or without buffer, Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD 14, CD20, CD11b, CD 16, HLA-DR, and CD8, In certain embodiments, it may be desirable to enrich for or positively select for regulatory T cells which typically express CD4⁺ₛ CD25⁺, CD62L¹″, GITR⁺, and FoxP3⁺. Alternatively, in certain embodiments, T regulatory cells are depleted by anti-C25 conjugated beads or other similar method of selection.

For isolation of a desired population of cells by positive or negative selection, the concentration of cells and surface {e.g., particles such as beads) can be varied. In certain embodiments, it may be desirable to significantly decrease the volume in which beads and cells are mixed together {i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present {i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression. In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles.

Whether prior to or after genetic modification of the T cells, the cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005. Additional strategies for expanding the population of T cells are described in, e.g., Dudley et al. Journal of Immunotherapy 2003; 26:332-42; Rasmussen et al., Journal of Immunological Methods 2010; 355:52-60; and Somerville et al., Journal of Translational Medicine 2012; 10:69. The entire contents of the foregoing patent documents are incorporated herein by reference.

(H) Administration of Autologous Cells

The autologous cells can be administered by any suitable route as known in the art. Preferably, the cells are administered as an intra-arterial or intravenous infusion, which lasts about 30 to about 60 minutes. Other exemplary routes of administration include intraperitoneal, intrathecal and intralymphatic.

Likewise, any suitable dose of autologous cells can be administered. For example, in one embodiment, from about 1.0×10⁸ cells to about 1.0×10¹² cells are administered. In one embodiment, from about 1.0×10¹⁰ cells to about 13.7×10¹⁰ T-cells are administered, with an average of around 5.0×10¹⁰ T-cells. Alternatively, in another embodiment, from about 1.2×10¹⁰ to about 4.3×10¹⁰ T-cells are administered.

In one embodiment, the autologous cells used for ACT are lymphocytes, e.g., T cells. In one embodiment, the T cells are “young” T cells, e.g., between 19-35 days old, as described in, for example, U.S. Pat. No. 8,383,099, incorporated by reference herein in its entirety. Young T cells are believed to have longer telomeres than older T cells, and longer telomere length may be associated with improved clinical outcome following ACT in some instances.

### Therapeutic Antibodies

In one embodiment, the extended-PK IL-2 can be used together with a therapeutic antibody. Accordingly, in one embodiment, subjects receiving ACT also receive extended-PK IL-2 and a therapeutic antibody. Administration of a therapeutic antibody to a subject receiving ACT and extended-PK IL-2 further enhances tumor regression and prolongs survival of the subject, relative to a subject receiving ACT and extended-PK IL-2.

Methods of producing antibodies, and antigen-binding fragments thereof, are well known in the art and are disclosed in, e.g., U.S. Pat. No. 7,247,301, US2008/0138336, and U.S. Pat. No. 7,923,221, all of which are herein incorporated by reference in their entirety.

Therapeutic antibodies that can be used in the methods of the present invention include, but are not limited to, any of the art-recognized anti-cancer antibodies that are approved for use, in clinical trials, or in development for clinical use. In some embodiments, more than one anti-cancer antibody can be included in the combination therapy of the present invention.

Non-limiting examples of anti-cancer antibodies include the following, without limitation:

trastuzumab (HERCEPTIN™. by Genentech, South San Francisco, Calif.), which is used to treat HER-2/neu positive breast cancer or metastatic breast cancer;

bevacizumab (AVASTIN™ by Genentech), which is used to treat colorectal cancer, metastatic colorectal cancer, breast cancer, metastatic breast cancer, non-small cell lung cancer, or renal cell carcinoma;

rituximab (RITUXAN™ by Genentech), which is used to treat non-Hodgkin's lymphoma or chronic lymphocytic leukemia;

pertuzumab (OMNITARG™ by Genentech), which is used to treat breast cancer, prostate cancer, non-small cell lung cancer, or ovarian cancer;

cetuximab (ERBITUX™ by ImClone Systems Incorporated, New York, N.Y.), which can be used to treat colorectal cancer, metastatic colorectal cancer, lung cancer, head and neck cancer, colon cancer, breast cancer, prostate cancer, gastric cancer, ovarian cancer, brain cancer, pancreatic cancer, esophageal cancer, renal cell cancer, prostate cancer, cervical cancer, or bladder cancer;

IMC-1C11 (ImClone Systems Incorporated), which is used to treat colorectal cancer, head and neck cancer, as well as other potential cancer targets;

tositumomab and tositumomab and iodine 1¹³¹ (BEXXAR™ by Corixa Corporation, Seattle, Wash.), which is used to treat non-Hodgkin's lymphoma, which can be CD20 positive, follicular, non-Hodgkin's lymphoma, with and without transformation, whose disease is refractory to Rituximab and has relapsed following chemotherapy;

In¹¹¹ ibirtumomab tiuxetan; Y⁹⁰ ibirtumomab tiuxetan; In¹¹¹ ibirtumomab tiuxetan and Y⁹⁰ ibirtumomab tiuxetan (ZEVALIN™ by Biogen Idec, Cambridge, Mass.), which is used to treat lymphoma or non-Hodgkin's lymphoma, which can include relapsed follicular lymphoma; relapsed or refractory, low grade or follicular non-Hodgkin's lymphoma; or transformed B-cell non-Hodgkin's lymphoma;

EMD 7200 (EMD Pharmaceuticals, Durham, N.C.), which is used for treating for treating non-small cell lung cancer or cervical cancer;

SGN-30 (a genetically engineered monoclonal antibody targeted to CD30 antigen by Seattle Genetics, Bothell, Wash.), which is used for treating Hodgkin's lymphoma or non-Hodgkin's lymphoma;

SGN-15 (a genetically engineered monoclonal antibody targeted to a Lewisγ-related antigen that is conjugated to doxorubicin by Seattle Genetics), which is used for treating non-small cell lung cancer;

SGN-33 (a humanized antibody targeted to CD33 antigen by Seattle Genetics), which is used for treating acute myeloid leukemia (AML) and myelodysplastic syndromes (MDS);

SGN-40 (a humanized monoclonal antibody targeted to CD40 antigen by Seattle Genetics), which is used for treating multiple myeloma or non-Hodgkin's lymphoma;

SGN-35 (a genetically engineered monoclonal antibody targeted to a CD30 antigen that is conjugated to auristatin E by Seattle Genetics), which is used for treating non-Hodgkin's lymphoma;

SGN-70 (a humanized antibody targeted to CD70 antigen by Seattle Genetics), that is used for treating renal cancer and nasopharyngeal carcinoma;

SGN-75 (a conjugate comprised of the SGN70 antibody and an Auristatin derivative by Seattle Genetics); and

SGN-17/19 (a fusion protein containing antibody and enzyme conjugated to melphalan prodrug by Seattle Genetics), which is used for treating melanoma or metastatic melanoma.

It should be understood that the therapeutic antibodies to be used in the methods of the present invention are not limited to those described supra. For example, the following approved therapeutic antibodies can also be used in the methods of the invention: brentuximab vedotin (ADCETRIS™) for anaplastic large cell lymphoma and Hodgkin lymphoma, ipilimumab (MDX-101; YERVOY™) for melanoma, ofatumumab (ARZERRA™) for chromic lymphocytic leukemia, panitumumab (VECTIBIX™) for colorectal cancer, alemtuzumab (CAMPATH™) for chronic lymphocytic leukemia, ofatumumab (ARZERRA™) for chronic lymphocytic leukemia, gemtuzumab ozogamicin (MYLOTARG™) for acute myelogenous leukemia.

Antibodies for use in the present invention can also target molecules expressed by immune cells, such as, but not limited to, tremelimumab (CP-675,206) and ipilimumab (MDX-010) which targets CTLA4 and has the effect of tumor rejection, protection from rechallenge, and enhanced tumor-specific T cell responses; OX86 which targets OX40 and increases antigen-specific CD8+ T cells at tumor sites and enhances tumor rejection; CT-011 which targets PD 1 and has the effect of maintaining and expanding tumor specific memory T cells and activates NK cells; BMS-663513 which targets CD137 and causes regression of established tumors, as well as the expansion and maintenance of CD8+ T cells, and daclizumab (ZENAPAX™) which targets CD25 and causes transient depletion of CD4+CD25+FOXP3+Tregs and enhances tumor regression and increases the number of effector T cells. A more detailed discussion of these antibodies can be found in, e.g., Weiner et al., Nature Rev. Immunol (2010); 10:317-27.

Other therapeutic antibodies can be identified that target tumor antigens. For example, the following tumor antigens can be targeted by therapeutic antibodies that may be administered in combination with ACT.

The tumor antigen may be an epithelial cancer antigen, (e.g., breast, gastrointestinal, lung), a prostate specific cancer antigen (PSA) or prostate specific membrane antigen (PSMA), a bladder cancer antigen, a lung (e.g., small cell lung) cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen. In another embodiment, the tumor antigen is a lymphoma antigen (e.g., non-Hodgkin's lymphoma or Hodgkin's lymphoma), a B-cell lymphoma cancer antigen, a leukemia antigen, a myeloma (i.e., multiple myeloma or plasma cell myeloma) antigen, an acute lymphoblastic leukemia antigen, a chronic myeloid leukemia antigen, or an acute myelogenous leukemia antigen. It should be understood that the described tumor antigens are only exemplary and that any tumor antigen can be targeted in the present invention.

In another embodiment, the tumor antigen is a mucin-1 protein or peptide (MUC-1) that is found on most or all human adenocarcinomas: pancreas, colon, breast, ovarian, lung, prostate, head and neck, including multiple myelomas and some B cell lymphomas. Patients with inflammatory bowel disease, either Crohn's disease or ulcerative colitis, are at an increased risk for developing colorectal carcinoma. MUC-1 is a type I transmembrane glycoprotein. The major extracellular portion of MUC-1 has a large number of tandem repeats consisting of 20 amino acids which comprise immunogenic epitopes. In some cancers it is exposed in an unglycosylated form that is recognized by the immune system (Gendler et al., J Biol Chem 1990; 265:15286-15293). In another embodiment, the tumor antigen is a mutated B-Raf antigen, which is associated with melanoma and colon cancer. The vast majority of these mutations represent a single nucleotide change of T-A at nucleotide 1796 resulting in a valine to glutamic acid change at residue 599 within the activation segment of B-Raf. Raf proteins are also indirectly associated with cancer as effectors of activated Ras proteins, oncogenic forms of which are present in approximately one-third of all human cancers. Normal non-mutated B-Raf is involved in cell signaling, relaying signals from the cell membrane to the nucleus. The protein is usually only active when needed to relay signals. In contrast, mutant B-Raf has been reported to be constantly active, disrupting the signaling relay (Mercer and Pritchard, Biochim Biophys Acta (2003) 1653(1):25-40; Sharkey et al., Cancer Res. (2004) 64(5):1595-1599).

In one embodiment, the tumor antigen is a human epidermal growth factor receptor-2 (HER-2/neu) antigen. Cancers that have cells that overexpress HER-2/neu are referred to as HER-2/neu⁺ cancers. Exemplary HER-2/neu⁺ cancers include prostate cancer, lung cancer, breast cancer, ovarian cancer, pancreatic cancer, skin cancer, liver cancer (e.g., hepatocellular adenocarcinoma), intestinal cancer, and bladder cancer.

HER-2/neu has an extracellular binding domain (ECD) of approximately 645 aa, with 40% homology to epidermal growth factor receptor (EGFR), a highly hydrophobic transmembrane anchor domain (TMD), and a carboxyterminal intracellular domain (ICD) of approximately 580 aa with 80% homology to EGFR. The nucleotide sequence of HER-2/neu is available at GENBANK™. Accession Nos. AH002823 (human HER-2 gene, promoter region and exon 1); M16792 (human HER-2 gene, exon 4): M16791 (human HER-2 gene, exon 3); M16790 (human HER-2 gene, exon 2); and M16789 (human HER-2 gene, promoter region and exon 1). The amino acid sequence for the HER-2/neu protein is available at GENBANK™. Accession No. AAA58637. Based on these sequences, one skilled in the art could develop HER-2/neu antigens using known assays to find appropriate epitopes that generate an effective immune response. Exemplary HER-2/neu antigens include p369-377 (a HER-2/neu derived HLA-A2 peptide); dHER2 (Corixa Corporation); li-Key MHC class II epitope hybrid (Generex Biotechnology Corporation); peptide P4 (amino acids 378-398); peptide P7 (amino acids 610-623); mixture of peptides P6 (amino acids 544-560) and P7; mixture of peptides P4, P6 and P7; HER2 [9₇₅₄]; and the like.

In one embodiment, the tumor antigen is an epidermal growth factor receptor (EGFR) antigen. The EGFR antigen can be an EGFR variant 1 antigen, an EGFR variant 2 antigen, an EGFR variant 3 antigen and/or an EGFR variant 4 antigen. Cancers with cells that overexpress EGFR are referred to as EGFR⁺ cancers. Exemplary EGFR⁺ cancers include lung cancer, head and neck cancer, colon cancer, colorectal cancer, breast cancer, prostate cancer, gastric cancer, ovarian cancer, brain cancer and bladder cancer.

In one embodiment, the tumor antigen is a vascular endothelial growth factor receptor (VEGFR) antigen. VEGFR is considered to be a regulator of cancer-induced angiogenesis. Cancers with cells that overexpress VEGFR are called VEGFR⁺ cancers. Exemplary VEGFR⁺ cancers include breast cancer, lung cancer, small cell lung cancer, colon cancer, colorectal cancer, renal cancer, leukemia, and lymphocytic leukemia.

In one embodiment the tumor antigen is prostate-specific antigen (PSA) and/or prostate-specific membrane antigen (PSMA) that are prevalently expressed in androgen-independent prostate cancers.

In another embodiment, the tumor antigen is Gp-100 Glycoprotein 100 (gp 100) is a tumor-specific antigen associated with melanoma.

In one embodiment, the tumor antigen is a carcinoembryonic (CEA) antigen. Cancers with cells that overexpress CEA are referred to as CEA⁺ cancers. Exemplary CEA⁺ cancers include colorectal cancer, gastric cancer and pancreatic cancer. Exemplary CEA antigens include CAP-1 (i.e., CEA aa 571-579), CAP1-6D, CAP-2 (i.e., CEA aa 555-579), CAP-3 (i.e., CEA aa 87-89), CAP-4 (CEA aa 1-11), CAP-5 (i.e., CEA aa 345-354), CAP-6 (i.e., CEA aa 19-28) and CAP-7.

In one embodiment, the tumor antigen is carbohydrate antigen 10.9 (CA 19.9). CA 19.9 is an oligosaccharide related to the Lewis A blood group substance and is associated with colorectal cancers.

In another embodiment, the tumor antigen is a melanoma cancer antigen. Melanoma cancer antigens are useful for treating melanoma. Exemplary melanoma cancer antigens include MART-1 (e.g., MART-1 26-35 peptide, MART-1 27-35 peptide); MART-1/Melan A; pMe117; pMe117/gp100; gp100 (e.g., gp 100 peptide 280-288, gp 100 peptide 154-162, gp 100 peptide 457-467); TRP-1; TRP-2; NY-ESO-1; p16; beta-catenin; mum-1; and the like.

In one embodiment, the tumor antigen is a mutant or wild type ras peptide. The mutant ras peptide can be a mutant K-ras peptide, a mutant N-ras peptide and/or a mutant H-ras peptide. Mutations in the ras protein typically occur at positions 12 (e.g., arginine or valine substituted for glycine), 13 (e.g., asparagine for glycine), 61 (e.g., glutamine to leucine) and/or 59. Mutant ras peptides can be useful as lung cancer antigens, gastrointestinal cancer antigens, hepatoma antigens, myeloid cancer antigens (e.g., acute leukemia, myelodysplasia), skin cancer antigens (e.g., melanoma, basal cell, squamous cell), bladder cancer antigens, colon cancer antigens, colorectal cancer antigens, and renal cell cancer antigens.

In another embodiment of the invention, the tumor antigen is a mutant and/or wildtype p53 peptide. The p53 peptide can be used as colon cancer antigens, lung cancer antigens, breast cancer antigens, hepatocellular carcinoma cancer antigens, lymphoma cancer antigens, prostate cancer antigens, thyroid cancer antigens, bladder cancer antigens, pancreatic cancer antigens and ovarian cancer antigens.

Other relevant cancer antigens include those disclosed in Cheever et al. (supra).

The foregoing mention of exemplary tumor antigens targeted by therapeutic antibodies is not intended to be limiting. Identifying therapeutic antibodies that recognize a tumor antigen of interest is within the ability of a person of ordinary skill

The therapeutic antibody can be a fragment of an antibody (e.g., a Fab, a scFv, a diabody); a complex comprising an antibody; or a conjugate comprising an antibody. The antibody can optionally be chimeric, humanized or fully human.

### Methods of Making Extended-PK IL-2 Proteins

In some aspects, the extended-PK IL-2 proteins of the invention are made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The invention also includes a vector capable of expressing the peptides in an appropriate host. The vector comprises the DNA molecule that codes for the peptides operatively linked to appropriate expression control sequences. Methods of affecting this operative linking, either before or after the DNA molecule is inserted into the vector, are well known. Expression control sequences include promoters, activators, enhancers, operators, ribosomal nuclease domains, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides since it is the most cost-effective method of making small peptides. Compounds that contain derivatized peptides or which contain non-peptide groups may be synthesized by well-known organic chemistry techniques.

Other methods of molecule expression/synthesis are generally known in the art to one of ordinary skill

### Pharmaceutical Compositions and Modes of Administration

In certain embodiments, extended-PK IL-2 is administered together (simultaneously or sequentially) with ACT and/or a therapeutic antibody. In certain embodiments, extended-PK IL-2 is administered prior to the administration of ACT and/or a therapeutic antibody. In certain embodiments, extended-PK IL-2 is administered concurrent with the administration of ACT and/or a therapeutic antibody. In certain embodiments, extended-PK IL-2 is administered subsequent to the administration of ACT and/or a therapeutic antibody. In certain embodiments, the extended-PK IL-2, ACT, and/or a therapeutic antibody, are administered simultaneously. In other embodiments, the extended-PK IL-2, ACT, and/or a therapeutic antibody, are administered sequentially. In yet other embodiments, the extended-PK IL-2, ACT, and/or a therapeutic antibody, are administered within one, two, or three days of each other. In a specific embodiment, ACT is administered first, and followed by a regimen of a therapeutic antibody and/or extended-PK IL-2 (e.g., Fc/IL-2). In certain embodiments, the therapeutic antibody and/or extended-PK IL-2 are administered, for example, once per week, twice per week, once per month, or twice per month. In certain embodiments, ACT is administered along with extended-PK IL2. The dosing schedule for the therapeutic antibody and extended-PK IL-2, when used together, will vary not only on the particular compounds or compositions selected, but also with the route of administration, the nature of the condition being treated, and the age and condition of the patient, and will ultimately be at the discretion of the patient's physician or pharmacist. The length of time during which the compounds used in the instant method will be given varies on an individual basis.

In certain embodiments, ACT and/or a therapeutic antibody is combined with continuous infusion of IL-2 in order to achieve continuous exposure to IL-2. Methods for continuous infusion are standard in the art, and protocols for continuous infusion of IL-2 are described in, e.g., Legha et al., Cancer 1996; 77:89-96 and Dillman et al., Cancer 1993; 71:2358-70.

In some embodiments, additional therapeutic agents are administered to a subject receiving extended-PK IL-2, ACT, and optionally a therapeutic antibody. Non-limiting examples of additional agents include GM-CSF (expands monocyte and neutrophil population), IL-7 (important for generation and survival of memory T-cells), interferon alpha, tumor necrosis factor alpha, IL-12, and therapeutic antibodies, such as anti-PD-1, anti-PD-L, anti-CTLA4, anti-CD40, anti-OX45, and anti-CD137 antibodies. In some embodiments, the subject receives extended-PK IL-2 and one or more therapeutic agents during a same period of prevention, occurrence of a disorder, and/or period of treatment.

Pharmaceutical compositions of the invention can be administered in combination therapy, i.e., combined with other agents. Agents include, but are not limited to, in vitro synthetically prepared chemical compositions, antibodies, antigen binding regions, and combinations and conjugates thereof. In certain embodiments, an agent can act as an agonist, antagonist, allosteric modulator, or toxin.

In certain embodiments, the invention provides for separate pharmaceutical compositions comprising extended-PK IL-2 with a pharmaceutically acceptable diluent, carrier, solubilizer, emulsifier, preservative and/or adjuvant and optionally a separate pharmaceutical composition comprising one or more therapeutic agents, such as a therapeutic antibody, with a pharmaceutically acceptable diluent, carrier, solubilizer, emulsifier, preservative and/or adjuvant.

In certain embodiments, the invention provides for pharmaceutical compositions comprising extended-PK IL-2 and one or more therapeutic agents, such as a therapeutic antibody, and optionally a therapeutically effective amount of at least one additional therapeutic agent, together with a pharmaceutically acceptable diluent, carrier, solubilizer, emulsifier, preservative and/or adjuvant, and another pharmaceutical composition comprises one or more therapeutic agents, e.g., a therapeutic antibody, together with a pharmaceutically acceptable diluent, carrier, solubilizer, emulsifier, preservative and/or adjuvant. In some embodiments, each of the agents, e.g., extended-PK IL-2, therapeutic antibody, and the optional additional therapeutic agent can be formulated as separate compositions.

In certain embodiments, acceptable formulation materials preferably are nontoxic to recipients at the dosages and concentrations employed. In some embodiments, the formulation material(s) are for s.c. and/or I.V. administration. In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In some embodiments, the formulation comprises PBS; 20 mM NaOAC, pH 5.2, 50 mM NaCl; and/or 10 mM NAOAC, pH 5.2, 9% Sucrose.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of extended-PK IL-2 and one or more therapeutic agents.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising extended-PK IL-2 and one or more therapeutic antibodies, with or without one or more therapeutic agents, can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising extended-PK IL-2 and optionally one or more therapeutic antibodies, with or without one or more therapeutic agents, can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising a desired extended-PK IL-2 and optionally one or more therapeutic agents, such as a therapeutic antibody, in a pharmaceutically acceptable vehicle. In certain embodiments, a vehicle for parenteral injection is sterile distilled water in which extended-PK IL-2 and optionally one or more therapeutic agents, such as a therapeutic antibody, are formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, extended-PK IL-2 and optionally one or more therapeutic agents, such as a therapeutic antibody, can be formulated as a dry powder for inhalation. In certain embodiments, an inhalation solution comprising extended-PK IL-2 and optionally one or more therapeutic agents, such as a therapeutic antibody, can be formulated with a propellant for aerosol delivery. In certain embodiments, solutions can be nebulized. Pulmonary administration is further described in PCT application no. PCT/US94/001875, which describes pulmonary delivery of chemically modified proteins.

In certain embodiments, it is contemplated that formulations can be administered orally. In certain embodiments, extended-PK IL-2 and optionally one or more therapeutic agents, such as a therapeutic antibody, that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of extended-PK IL-2 and, optionally, one or more therapeutic agents, such as a therapeutic antibody. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of extended-PK IL-2 and optionally one or more therapeutic agents, such as a therapeutic antibody, in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving extended-PK IL-2 and optionally one or more therapeutic agents, such as a therapeutic antibody, in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions can also include liposomes, which can be prepared by any of several methods known in the art. See, e.g., Eppstein et al., Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising extended-PK IL-2 and optionally one or more pharmaceutical compositions comprising therapeutic agents, such as a therapeutic antibody, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which extended-PK IL-2 is being used, the route of administration, and the size (body weight, body surface or organ size) and/or condition (the age and general health) of the patient. In certain embodiments, the clinician can titer the dosage and modify the route of administration to obtain the optimal therapeutic effect. In certain embodiments, a typical dosage can range from about 0.1 μg/kg to up to about 100 mg/kg or more, depending on the factors mentioned above. In certain embodiments, the dosage can range from 0.1 μg/kg up to about 100 mg/kg; or 1 μg/kg up to about 100 mg/kg; or 5 μg/kg up to about 100 mg/kg.

In certain embodiments, the frequency of dosing will take into account the pharmacokinetic parameters of extended-PK IL-2 in the formulation used. In certain embodiments, a clinician will administer the composition until a dosage is reached that achieves the desired effect. In certain embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of the combination therapy may be administered by different routes.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

In certain embodiments, it can be desirable to use a pharmaceutical composition comprising extended-PK IL-2 and optionally one or more therapeutic agents, such as a therapeutic antibody in an ex vivo manner. In such instances, cells, tissues and/or organs that have been removed from the patient are exposed to a pharmaceutical composition comprising extended-PK IL-2 and optionally one or more therapeutic agents, such as a therapeutic antibody, after which the cells, tissues and/or organs are subsequently implanted back into the patient.

In certain embodiments, extended-PK IL-2 and optionally one or more therapeutic agents, such as a therapeutic antibody, can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### Kits

A kit can include extended-PK IL-2 and, optionally, one or more therapeutic agents, such as a therapeutic antibody, disclosed herein and instructions for use. The kits may comprise, in a suitable container, extended-PK IL-2 and, optionally, one or more therapeutic agents, such as a therapeutic antibody, one or more controls, and various buffers, reagents, enzymes and other standard ingredients well known in the art.

The container can include at least one vial, well, test tube, flask, bottle, syringe, or other container means, into which extended-PK IL-2 and, optionally, one or more therapeutic agents, such as a therapeutic antibody, may be placed, and in some instances, suitably aliquoted. Where an additional component is provided, the kit can contain additional containers into which this component may be placed. The kits can also include a means for containing extended-PK IL-2 and, optionally, one or more therapeutic agents, such as a therapeutic antibody, and any other reagent containers in close confinement for commercial sale. Such containers may include injection or blow-molded plastic containers into which the desired vials are retained. Containers and/or kits can include labeling with instructions for use and/or warnings.

### Methods of Treatment

The extended-PK IL-2 and one or more therapeutic agents, such as a therapeutic antibody, and/or nucleic acids expressing them, are useful for treating a disorder associated with abnormal apoptosis or a differentiative process (e.g., cellular proliferative disorders or cellular differentiative disorders, such as cancer). Non-limiting examples of cancers that are amenable to treatment with the methods of the present invention are described below. Extended-PK IL-2, wherein the IL-2 moiety is wild-type IL-2, is an exemplary molecule for use in the methods of the invention.

Examples of cellular proliferative and/or differentiative disorders include cancer (e.g., carcinoma, sarcoma, metastatic disorders or hematopoietic neoplastic disorders, e.g., leukemias). A metastatic tumor can arise from a multitude of primary tumor types, including but not limited to those of prostate, colon, lung, breast and liver. Accordingly, the compositions of the present invention (e.g., extended-PK IL-2 and one or more therapeutic agents, such as a therapeutic antibody and/or the nucleic acid molecules that encode them) can be administered to a patient who has cancer. Extended-PK IL-2 and one or more therapeutic agents, such as a therapeutic antibody, can be used to treat a patient (e.g., a patient who has cancer) prior to, or simultaneously with, the administration of ex vivo expanded T cells.

As used herein, we may use the terms “cancer” (or “cancerous”), “hyperproliferative,” and “neoplastic” to refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The term “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus. With respect to the methods of the invention, the cancer can be any cancer, including any of acute lymphocytic cancer, acute myeloid leukemia, alveolar rhabdomyo sarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the vulva, chronic lymphocytic leukemia, chronic myeloid cancer, cervical cancer, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, peritoneum, omentum, and mesentery cancer, pharynx cancer, prostate cancer, rectal cancer, renal cancer, skin cancer, soft tissue cancer, testicular cancer, thyroid cancer, ureter cancer, urinary bladder cancer, and digestive tract cancer such as, e.g., esophageal cancer, gastric cancer, pancreatic cancer, stomach cancer, small intestine cancer, gastrointestinal carcinoid tumor, cancer of the oral cavity, colon cancer, and hepatobiliary cancer. A preferred cancer is melanoma. A particularly preferred cancer is metastatic melanoma.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The mutant IL-2 polypeptides can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Sternberg disease.

It will be appreciated by those skilled in the art that amounts for each of the extended-PK IL-2 and the one or more therapeutic agents, such as a therapeutic antibody, that are sufficient to reduce tumor growth and size, or a therapeutically effective amount, will vary not only on the particular compounds or compositions selected, but also with the route of administration, the nature of the condition being treated, and the age and condition of the patient, and will ultimately be at the discretion of the patient's physician or pharmacist. The length of time during which the compounds used in the instant method will be given varies on an individual basis.

It will be appreciated by those skilled in the art that the B16 melanoma model used herein is a generalized model for solid tumors. That is, efficacy of treatments in this model is also predictive of efficacy of the treatments in other non-melanoma solid tumors. For example, as described in Baird et al. (J Immunology 2013; 190:469-78; Epub Dec. 7, 2012), efficacy of cps, a parasite strain that induces an adaptive immune resposnse, in mediating anti-tumor immunity against B16F10 tumors was found to be generalizable to other solid tumors, including models of lung carcinoma and ovarian cancer. In another example, results from a line of research into VEGF targeting lymphocytes also shows that results in B16F10 tumors were generalizable to the other tumor types studied (Chinnasamy et al., JCI 2010; 120:3953-68; Chinnasamy et al., Clin Cancer Res 2012; 18:1672-83). In yet another example, immunotherapy involving LAG-3 and PD-1 led to reduced tumor burden, with generalizable results in a fibro sarcoma and colon adenocarcinoma cell lines (Woo et al., Cancer Res 2012; 72:917-27).

It will be appreciated by those skilled in the art that reference herein to treatment extends to prophylaxis as well as the treatment of the noted cancers and symptoms.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for. The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992). Moreover, while the examples below employ extended-PK IL-2 of mouse origin, it should be understood that corresponding human extended-PK IL-2 can be readily generated by those of ordinary skill in the art using methods described supra, and used in the methods of the present invention.

### Example 1

### Generation of High Affinity CD25-Binding IL-2 Mutants

The generation and testing of extended-PK IL-2 was described in International Patent Application No. PCT/US2013/042057, filed May 21, 2013, and claiming priority to U.S. Provisional Patent Application No. 61/650,277, filed May 22, 2012. The entire contents of the foregoing applications are incorporated by reference herein. Examples 1-7 below summarize the generation and testing of extended-PK IL-2 constructs.

Mouse IL-2 was affinity matured with error-prone PCR and yeast surface display to obtain high affinity CD25-binding IL-2 mutants. The mutagenesis approach and affinity maturation progress was determined by referencing a model of the mouse IL-2/IL-2R complex based on the crystal structure of the human IL-2/IL-2R complex. Error-prone PCR conditions (nucleotide analogue concentration and amplification cycle number) were chosen such as to produce one to two amino acid mutations per gene, distributed throughout the entire IL-2 gene.

A yeast surface display library was labeled with soluble CD25 and screened six times for higher affinity clones by FACS. Sequences from a selection of clones indicated accumulation of mutants that encode proline or threonine at position 126, which is serine in wild-type mouse IL-2. Notably, position 126 is proline or threonine in many other animal species. According to the model of the IL-2/IL-2 receptor complex, this position locates to the interface with CD25. Further affinity maturation of S126P and S126T IL-2, which bound to CD25 with an affinity 2 to 3-fold higher than wild-type IL-2, led to the generation of IL-2 mutants with 500-fold affinity improvement over wild-type IL-2. When these mutants were sequenced, their mutations were found to locate to two difference faces of IL-2, that in potential contact with CD25 and that in potential contact with IL-2Rβ.

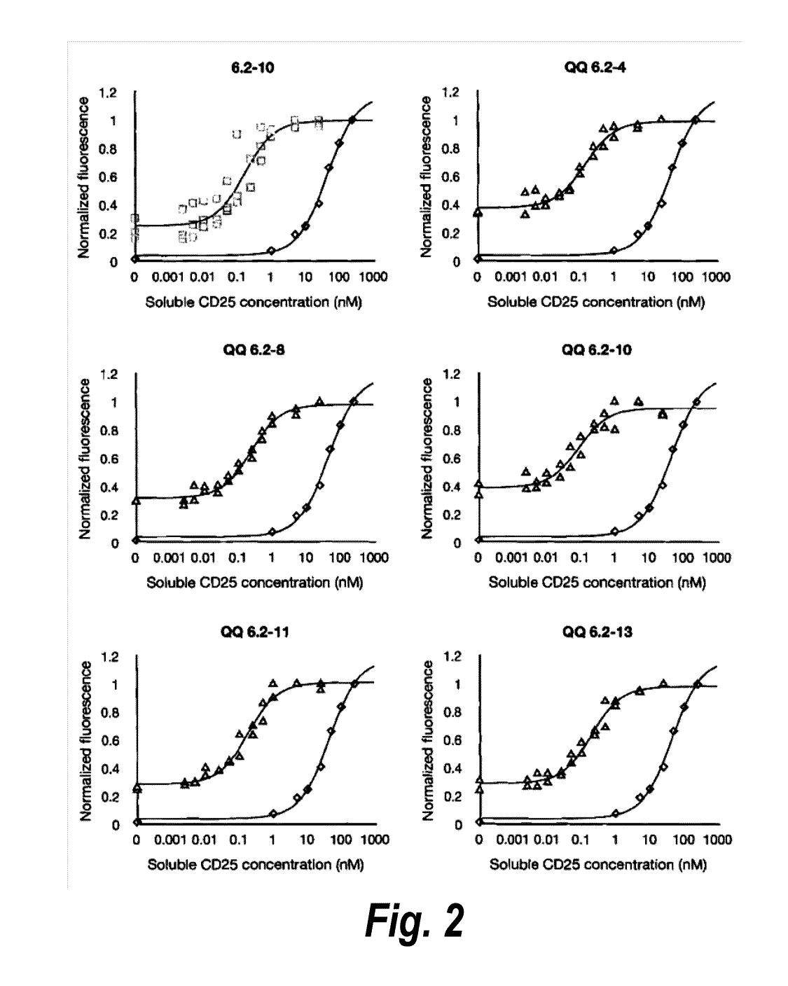

To avoid disrupting the interaction with IL-2Rβ, putative IL-2Rβ-binding mutations were mutated so as to revert the mutations back to the wild-type amino acid residues by site-directed mutagenesis. The mutants and their sequences are shown in FIG. 1. These reversion mutants retained high CD25 binding affinity (FIG. 2). For convenience, high-affinity CD25-binding QQ 6.2-10 (“QQ6210”) was used in further experiments.

### Example 2

### Generation of a Non-CD25-Binding IL-2 Mutant

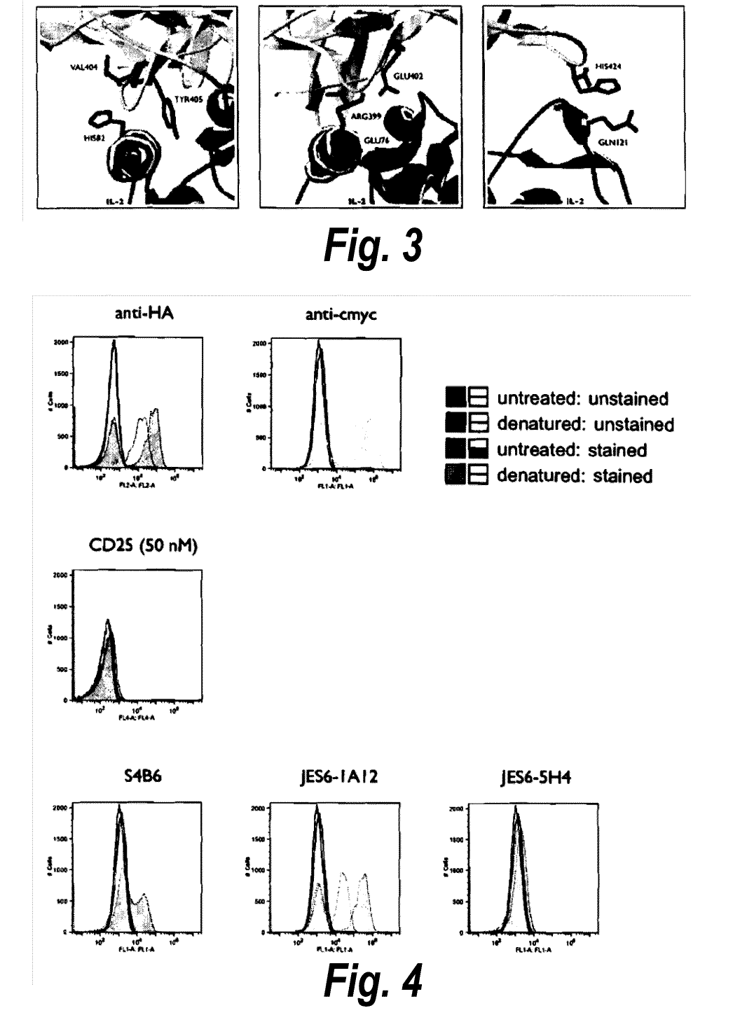

Inspection of the mouse IL-2/IL-2 receptor complex revealed three amino acid residues in intimate contact with CD25: E76, H82, and Q121 (FIG. 3). To disrupt CD25 binding, each of these residues was mutated to one of four alternative amino acids that differ from the wild-type in size, hydrophobicity, or charge. These 12 mutants were displayed on the surface of yeast and tested for CD25 binding by labeling with 5 or 50 nM soluble CD25.

TABLE 1
Mutations

E76 --> R, F, A, G
H82 --> E, S, A, G
Q121 --> R, S, A, G

While all H82 and Q121 mutants retained CD25 binding, no CD25 binding was detected for E76 mutants (FIG. 4). Labeling of E76 mutants with conformation-specific anti-mouse IL-2 antibodies, with or without thermal denaturation, suggested that E76A and E76G are well-folded proteins with no detectable binding at 50 nM soluble CD25 (FIG. 4).

### Example 3

### Fc/IL-2 and Mutants

A vector encoding the heavy chain of a mouse IgG2a from C57BL/6 mice was provided by J. Ravetch (The Rockefeller University). A fragment encoding the hinge, C_{H}2, and C_{H}3 domains was cloned into the gWIZ vector (Genlantis) from PstI to SalI sites. Mouse IL-2 with a 6×His tag was subsequently cloned into the vector C-terminal to Fc. To enable expression of monovalent Fc/IL-2, a vector encoding the Fc with a FLAG tag was also constructed. Notably, a D265A mutation was introduced into the Fc coding sequence to reduce effector function (i.e., to reduce ADCC and CDC) as disclosed in Baudino et al. (J Immunol 2008; 181:6664-9). DNA sequences were confirmed by DNA sequencing. Plasmid DNA was transformed into XL1-Blue for amplification. DNA was purified from cells using PureLink HiPure Maxiprep Kit (Invitrogen) and sterile filtered.

HEK293 cells (Invitrogen) were cultured according to manufacturer's instructions. gWIZ vectors encoding D265A Fc fused with IL-2 (nucleic acid sequence: SEQ ID NO: 11; amino acid sequence: SEQ ID NO: 12), QQ6210 (nucleic acid sequence: SEQ ID NO: 13; amino acid sequence: SEQ ID NO: 14), E76A IL-2 (nucleic acid sequence: SEQ ID NO: 15; amino acid sequence: SEQ ID NO: 16), or E76G IL-2 (nucleic acid sequence: SEQ ID NO: 17; amino acid sequence: SEQ ID NO: 18) were co-transfected with gWIZ D265A Fc FLAG, encoding D265AFc/flag (nucleic acid sequence: SEQ ID NO: 9; amino acid sequence: SEQ ID NO: 10), into HEK293 cells using PEI in FreeStyle 293 media supplemented with OptiPro (Invitrogen). Seven days post transfection, culture supernatants were harvested by centrifugation (30 min at 15,000×g, 4° C.) and the supernatant sterilized by filtration through 0.22 μm filters.

Monovalent Fc/IL-2 fusions were purified by sequential TALON His-tag metal affinity purification (Clontech) and anti-FLAG affinity chromatography (Sigma-Aldrich) following manufacturer's instructions. Elution fractions were concentrated using 15-ml 30-kDa Amicon Ultra Centrifugal Devices (Millipore) and buffered exchanged into PBS. Protein concentration was determined by the Beer-Lambert Law:

A=εlc,

where
A=absorbance at 280 nm,
ε=extinction coefficient,
l=path length, and
c=concentration
   Absorbance at 280 nm was measured using a NanoDrop 2000c (Thermo Scientific). The molecular weights and extinction coefficients of Fc/IL-2 fusion proteins were estimated from their amino acid sequences. Fc/IL-2 fusions were secreted using HEK293 cells and purified by sequential TALON resin and anti-FLAG affinity chromatography.

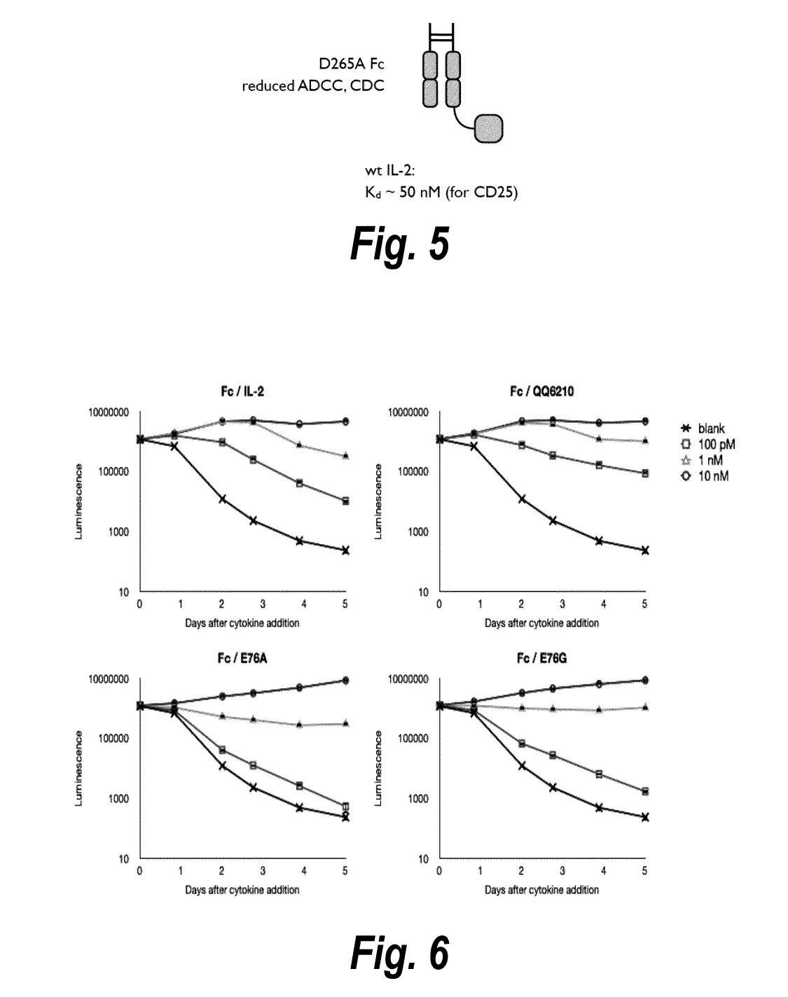

All Fc/IL-2 fusions used in the Examples described infra all have the D265A mutation in the Fc moiety (to reduce effector function, i.e., ADCC and CDC) and are in monovalent form (to separate any effects observed from that caused by IL-2 bivalency) (FIG. 5). Fc/IL-2 fusions need not be limited to the monovalent form, but can also be used in the bivalent form. The beta half-life of Fc/IL-2 is approximately 15 hours.

### Example 4

### Effects of Fc/IL-2 Fusions on Cell Proliferation of a Cytotoxic T Cell Line

To determine the effects of CD25 binding affinity on cell proliferation, the effects of an affinity series of mouse IL-2, consisting of Fc-fused high-affinity CD25-binding QQ 6.2-10 (“Fc/QQ6210”), wild-type IL-2 (“Fc/IL-2”), and a non-CD25 binding IL-2 mutant named E76G (“Fc/E76G”) were tested for the ability to stimulate cell proliferation. As described supra, these three Fc/IL-2 fusions have the D265A mutation in the Fc moiety.

| | Extinction | |
|---|---|---|
| | coefficient, ε | Molecular weight |
| Protein | (M⁻¹ cm⁻¹) | (g/mol) |
| Fc/IL-2 | 69870 | 72514.5 |
| Fc/QQ6210 | 68380 | 72592.4 |
| Fc/E76G | 69870 | 72442.4 |

To verify that Fc/IL-2, Fc/QQ6210, Fc/E76A, and Fc/E76G were functional, they were assayed for their ability to stimulate the growth of CTLL-2 cells, a murine cytotoxic T cell line. Under static conditions, all Fc/IL-2 proteins support CTLL-2 growth at 100 pM, 1 nM, and 10 nM (FIG. 6). The different growth kinetics resulting from stimulation with Fc/E76A and Fc/E76G likely reflects the lack of CD25 binding. For convenience, Fc/E76G was selected for further characterization in vivo.

### Example 5

### Fc/IL-2 Fusions Thereof Exhibit Extended Circulation Half-Life In Vivo

IL-2 has a very short systemic half-life, with an initial clearance phase with an alpha half-life of 12.9 min followed by a slower phase with a beta half-life of 85 min (Konrad et al., Cancer Res 1990; 50:2009-17). Thus, one of the difficulties associated with IL-2 therapy is the maintenance of therapeutic concentrations of IL-2 (1-100 pM) for a sustained period. To this end, the in vivo circulation half-lives of Fc/IL-2, Fc/QQ6210, and Fc/E76G were determined.

Each Fc/IL-2 fusion was labeled with IRDye 800 and injected intravenously into C57BL/6 mice as a 50 μg bolus. Blood samples were collected over four days. Serum levels of Fc/IL-2 fusions, as determined by the 800 nm signal within blood samples, was fitted to the biexponential decay equation MFI(t)=Ae^{-αt}+Be^{-βt}, where MFI is the mean fluorescence intensity of the blood sample, t is time, and A, B, α, and β are pharmacokinetic parameters to be fitted. As shown in Table 2, all Fc/IL-2 fusions exhibit substantially prolonged in vivo persistence compared to non-Fc fused IL-2.

TABLE 2

| | | | α | β | t_{1/2, α} | t_{1/2, β} |
|---|---|---|---|---|---|---|
| Protein | A | B | (hr⁻¹) | (hr⁻¹) | (hr) | (hr) |
| Fc/IL-2 | 0.50 ± | 0.70 ± | 0.12 ± | 0.05 ± | 1.9 ± | 16.4 ± |
| | 0.15 | 0.53 | 0.08 | 0.01 | 0.9 | 3.6 |
| Fc/QQ6210 | 0.44 ± | 0.07 ± | 0.19 ± | 0.02 ± | 3.6 ± | 34.3 ± |
| | 0.11 | 0.02 | 0.01 | 0.00 | 0.2 | 3.2 |
| Fc/E76G | 0.71 ± | 0.16 ± | 0.25 ± | 0.03 ± | 3.0 ± | 25.4 ± |
| | 0.05 | 0.02 | 0.06 | 0.00 | 0.7 | 1.8 |

### Example 6

### Fc/IL-2 and Mutants Induce Splenomegaly and Alter T Cell and NK Cell Composition

To determine the effects of Fc/IL-2, Fc/QQ6210, and Fc/E76G on T cell and NK cell composition in vivo, C57BL/6 mice were injected intravenously once with 5 or 25 μg Fc/IL-2, Fc/QQ6210, or Fc/E76G. Four days later, spleens were photographed and splenocytes analyzed for T and NK cell composition by FACS.

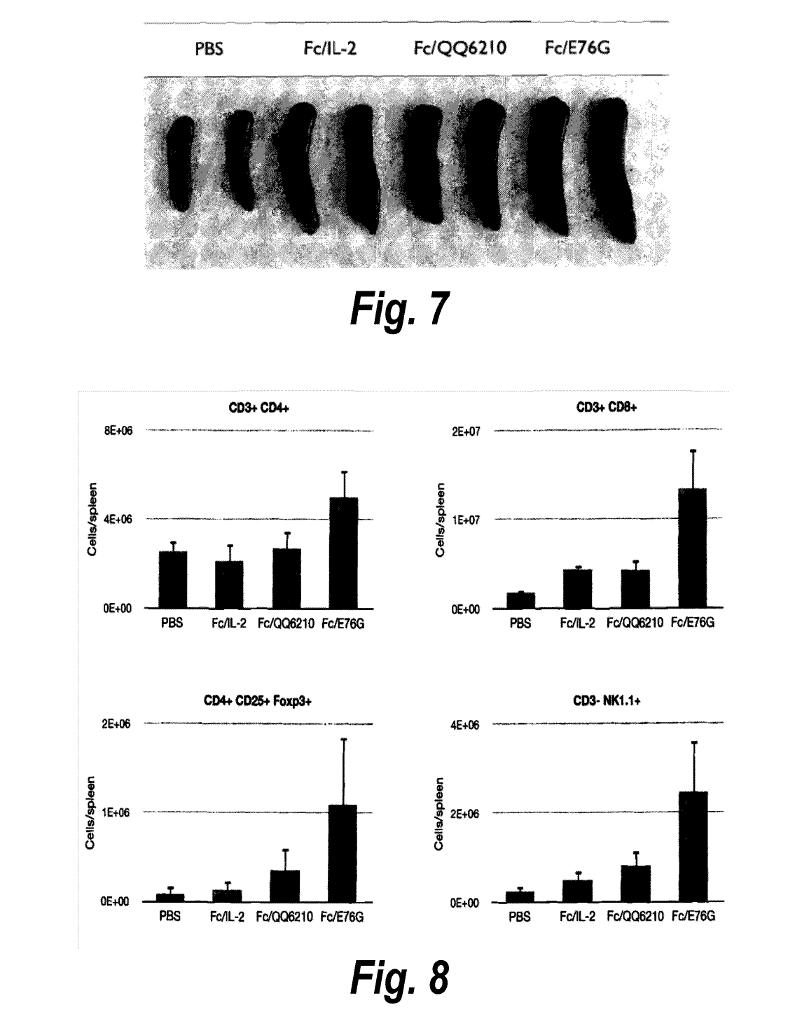

Both doses of Fc/IL-2 fusions increased spleen size compared to PBS-treated controls (FIG. 7). With respect to CD8+ T cell and NK cell composition, Fc/IL-2 and Fc/QQ6210 expanded CD8+ T cell and NK cells approximately 2-fold, while Fc/E76G expanded these populations up to 5-fold compared to PBS-treated controls (FIG. 8). The notable expansion of CD8+ T and NK cells by Fc/E76G validates the functional signaling of this mutant through IL-2Rβ and γ_{c}.

### Example 7

### Toxicity of Fc/IL-2 Fusions

Total animal weight was used as a proxy for toxicity, and lung wet weight was used as an indicator for pulmonary edema and vascular leak syndrome, which are often associated with IL-2 therapy.

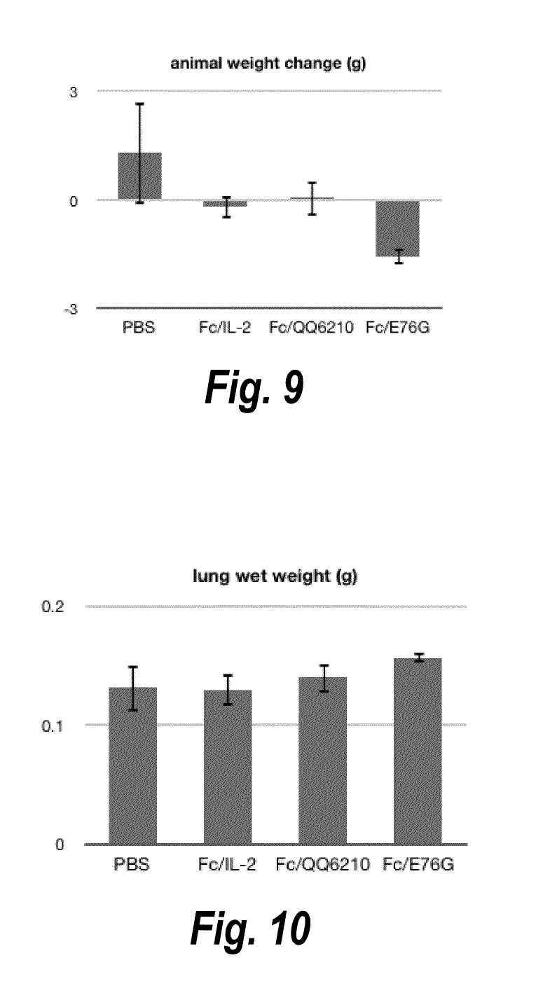

As shown in FIG. 9, Fc/IL-2 and Fc/QQ6210 were well tolerated at the two doses tested (5 μg and 25 μg), whereas Fc/E76G was highly toxic at 25 μg, likely because it strongly promoted CD8+ T cell and NK cell growth as described in Example 6. Fc/E76G was well tolerated at the lower dose of 5 μg.

Fc/IL-2 fusions did not significantly affect pulmonary wet weight compared to PBS-treated controls (FIG. 10). In contrast to a previous study by Krieg et al. (PNAS 2010; 107:11906-11), CD25 binding did not drive IL-2 toxicity in the lung, as demonstrated by the similar wet lung wet weight of mice injected with all three Fc/IL-2 fusions and the PBS control.

### Example 8

### The Pmel-1 Mouse Model for Adoptive Cell Therapy (ACT)

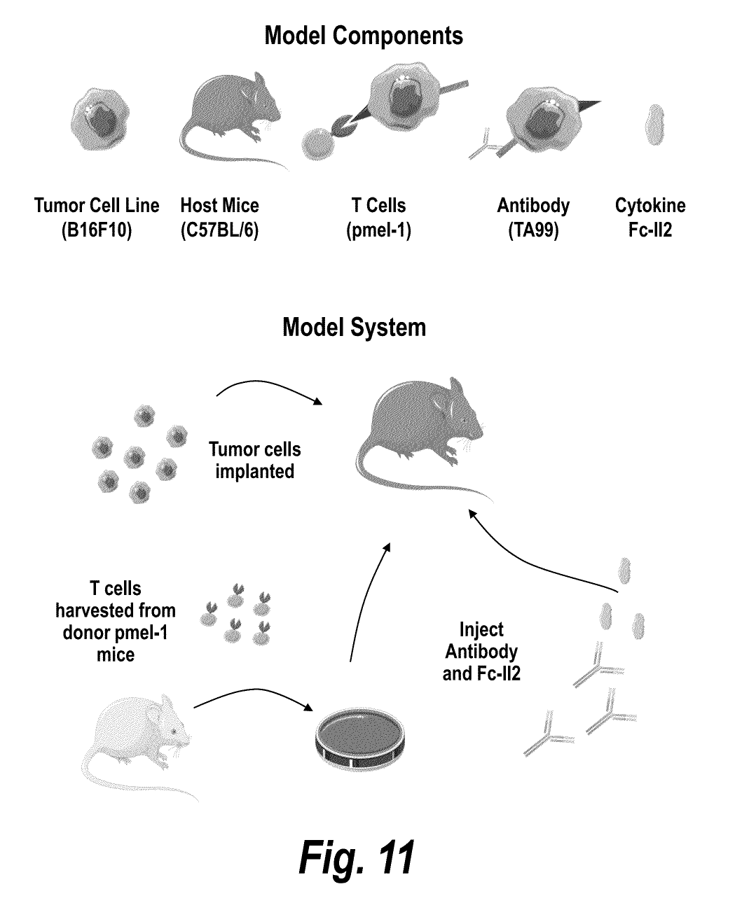

The pmel-1 mouse model represents a pre-clinical approximation of ACT. The components of this model are illustrated in FIG. 11. Pmel-1 is the designation of a transgenic mouse that serves a T cell donor. T cells obtained from this mouse are also referred to as “pmel-1,” or alternatively as “pmel-1 cells.” The B16F10 cell line is a poorly immunogenic melanoma that aggressively forms subcutaneous tumors when injected into the flanks of patient mice. Pmel-1 cells express a single TCR, which targets an MHC expressed peptide on the surface of melanin-producing cells, including B16F10. C57BL/6 is a host mouse strain that is allogeneic to both B16F10 and pmel-1 cells.

Fc-IL2 is an exemplary extended-PK IL-2 construct (described above) that promotes the activation of the immune system, including pmel-1 T cells, with enhanced pharmacokinetic properties.

The antibody TA99 targets another surface marker (TRP1) of melanin-producing cells, including B16F10.

### Example 9

### ACT Combination Therapy

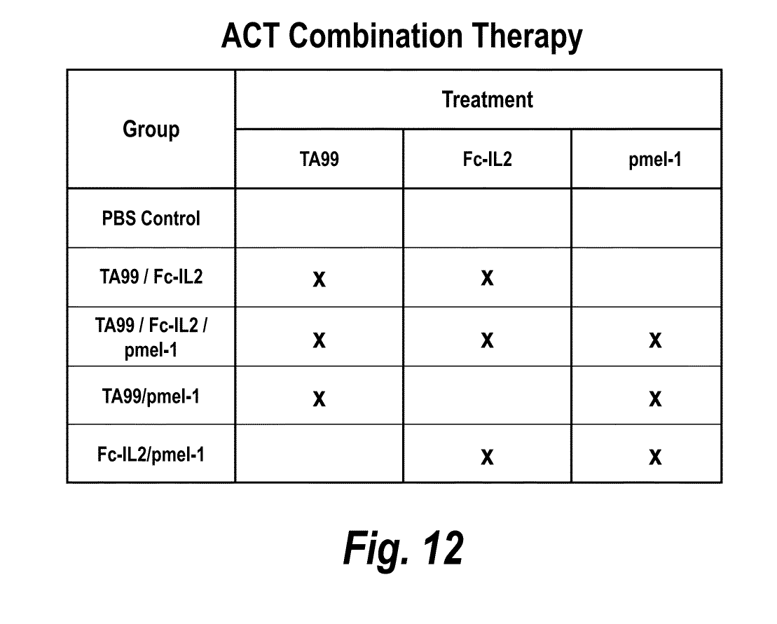

A study was conducted to examine the effect of Fc-IL-2 on ACT. The combination of Fc-IL-2, ACT, and a therapeutic antibody was also tested. Five groups of C57BL/6 host mice were separated into treatment groups as described in FIG. 12. All mice received a subcutaneous injection of 1e6 B16F10 melanoma cells.

The first group of mice (control) received PBS. The second group of mice received treatment with the TA99 antibody and Fc-IL-2. The third group of mice received the TA99 antibody, Fc-IL-2, and ACT with pmel-1 cells. The fourth group of mice received the TA99 antibody and ACT with pmel-1 cells. The fifth group of mice received Fc-IL-2 and ACT with pmel-1 cells. The Fc-IL-2 construct used in these experiments contained a D265A substitution in the Fc moiety, as described above.

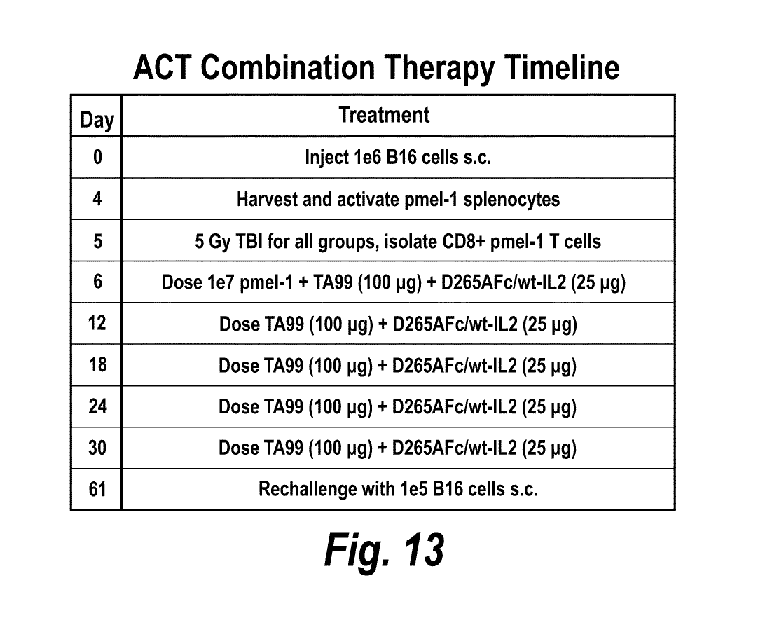

A timeline detailing the treatment regimen is depicted in FIG. 13. On day 0, all mice were injected with 1e6 B16F10 melanoma cells. On day 4, pmel-1 splenocytes were harvested from pmel-1 donors, and the harvested cells were activated. On day 5, all mice were preconditioned with 5 Gy of total body irradiation (TBI). This lymphodepletion creates a suitable environment for the establishment of transferred immune cells. CD8+ pmel-1 T cells were also isolated. On day 6, mice received their first course of treatment. This protocol allows for the establishment of fairly large tumors before treatment is initiated.

On day 6, mice received the TA99 antibody (100 μg; groups 2-4) and/or Fc-IL-2 (25 μg; groups 2, 3 and 5) (see FIG. 12). Mice in treatment groups receiving ACT were also administered 1e7 pmel-1 cells (groups 3-5). Mice in the control group (group 1) received PBS.

On day 12, day 18, day 24, and day 30, mice in the second and third groups received the TA99 antibody (100 μg) and Fc-IL-2 (25 μg). Mice in the fourth group received the TA99 antibody (100 μg). Mice in the fifth group received Fc-IL-2 (25 μg). Mice in the control group (group 1) received PBS.

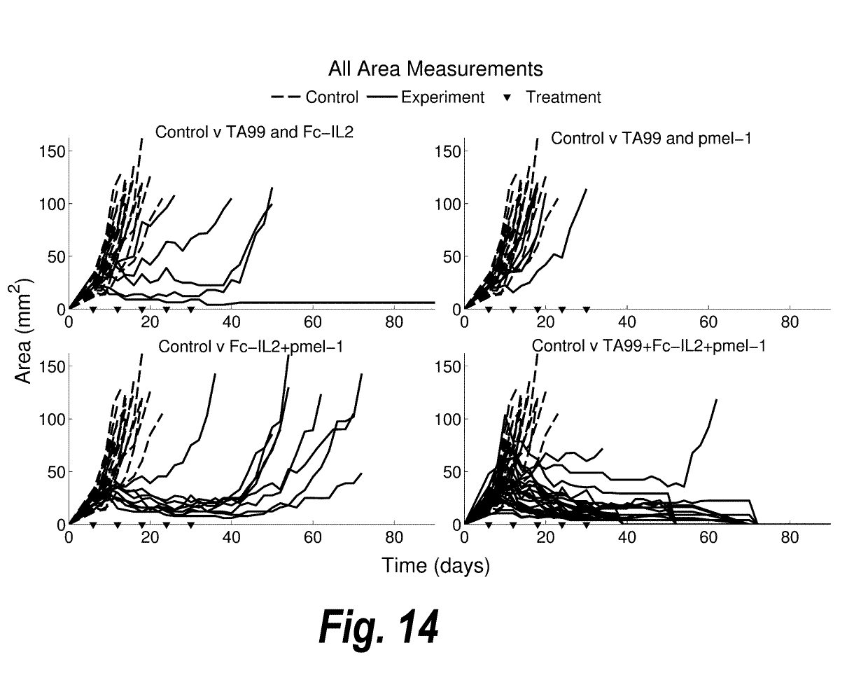

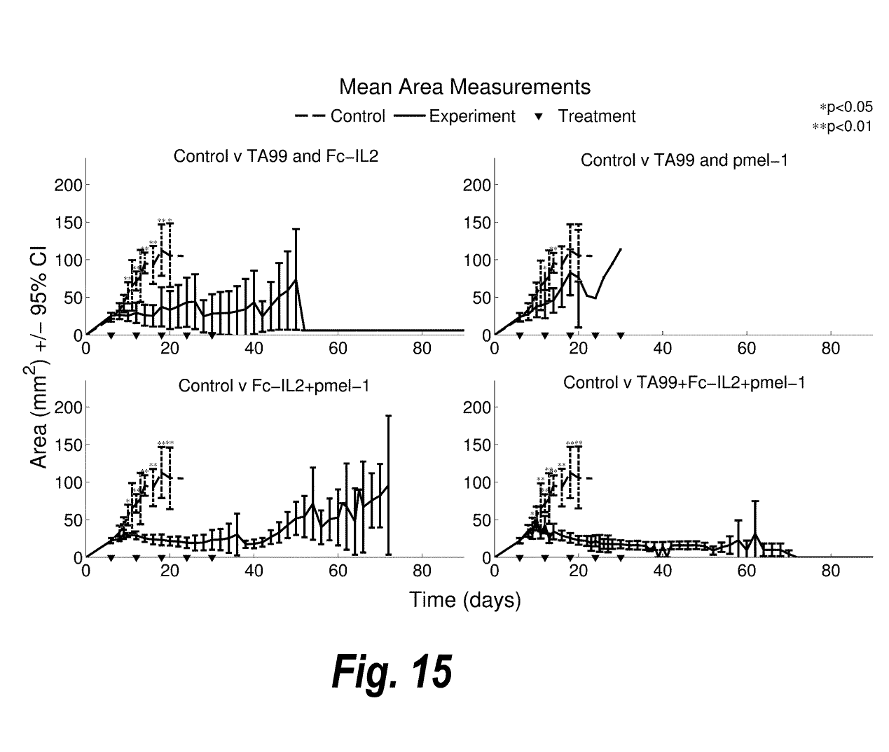

FIG. 14 presents the results of the foregoing experiment. Growth curves show the tumor area (mm²) at the respective number of days following injection with B16F10 cells. As shown therein, the combination of Fc-IL-2 and ACT led to a significant delay in tumor growth. The addition of the TA99 antibody to the treatment regimen leads to complete cures. (In cured mice, tumor areas never reach 0 due to residual pigmentation left over from the tumors. These tumor “scars” do not grow out, and can still be measured.) As monotherapies, these agents had only a modest effect on the growth of tumors. Antibody therapy (TA99) paired with ACT (pmel-1) had little increased benefit over ACT alone. Pairing ACT (pmel-1) with Fc-IL-2 significantly increased survival of treated mice. These results indicate that Fc-IL-2 significantly enhances the success of ACT therapy, and stimulates enhanced proliferation and survival of the transferred cells. The survival benefit of ACT in combination with Fc-IL-2 was significant enough to justify its use as a combination therapy in the absence of appropriate therapeutic antibodies. The combination of ACT (pmel-1), Fc-IL-2, and antibody therapy (TA99) was the most effective, leading to complete cures in 5/5 mice. After 60 days and signs of complete tumor remission, two mice from this group were re-challenged with B 16F10 cells, which failed to form any visible tumors. Such rejection of secondary tumor challenge indicates the establishment of immune memory, and increased persistence of transferred cells. FIG. 15 depicts the average tumor area and confidence intervals from the data shown in FIG. 14.

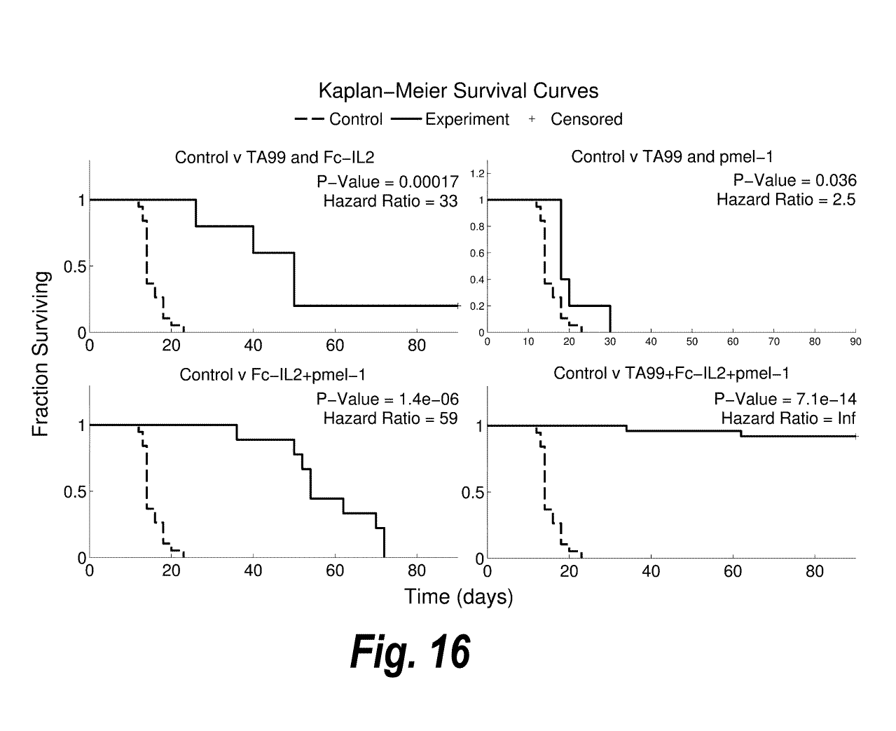

The fraction of mice surviving at each day following tumor challenge is presented in FIG. 16. The combination of ACT with Fc-IL-2 significantly extends the survival of treated mice. Addition of therapeutic antibody TA99 to the treatment regimen results in complete cures in 100% of treated mice.

### Example 10

### Fc-IL-2 Enhances Proliferation and Persistence of Transferred Cells

Pmel-1 mice have been crossed with mice expressing luciferase to create the strain pmel-1-luc. T cells from these mice produce bioluminescence when exposed to the D-luciferin molecule, allowing for the determination of in vivo location using imaging equipment.

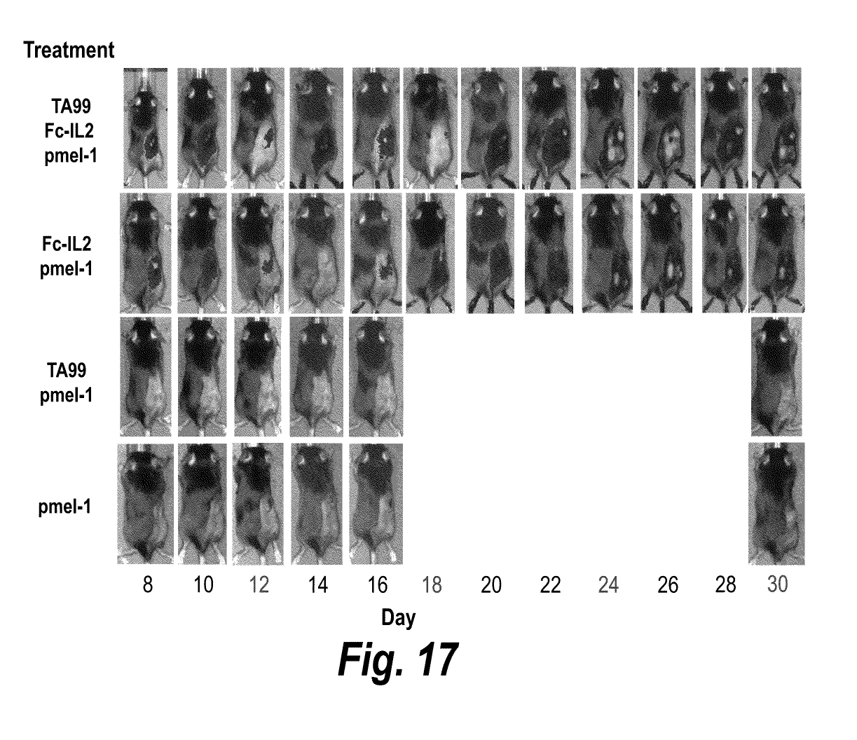

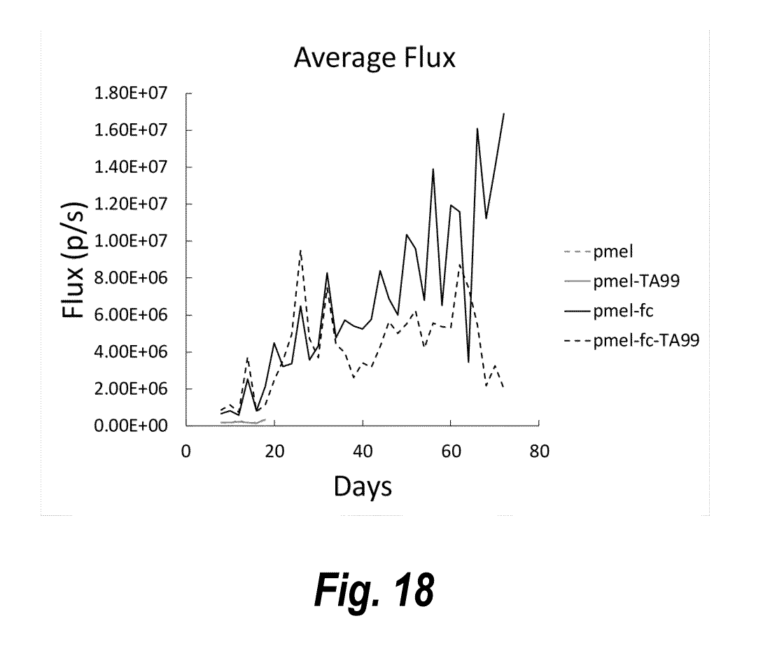

Mice were treated as described in Example 9 above. Briefly, on day O, C57BL/6 host mice were subcutaneously injected with 1e6 B16F10 melanoma cells. On day 5, mice were preconditioned with 5 Gy of total body irradiation. On day 6, mice were administered 1e7 pmel-1 cells obtained from pmel-1-luc mice. Mice were also administered Fc-IL-2, TA99, or a combination of Fc-IL-2 and TA99, as shown in FIG. 17. Fc-IL-2 (25 μg) and TA99 (100 μg) were administered by orbital injection on days 12, 18, 24, and 30. Bioluminescence indicative of transferred cells was imaged at various time points, as shown in FIG. 17. Transferred cells are shown in blue. As indicated in FIGS. 17 and 18, large quantities of transferred cells are visible in mice receiving TA99, Fc-IL-2, and pmel-1, and in mice receiving Fc-IL-2 and pmel-1. A significant amount of luminescence indicative of transferred cells is apparent at all time points examined, but is particularly prominent in the days following Fc-IL-2 administration. In contrast, very little luminescence is visible in mice receiving TA99 and pmel-1, or pmel-1 alone (in the absence of Fc-IL-2). As shown in FIG. 18, after treatment with TA99, Fc-IL-2, and pmel-1), mice show a decline in signal as the tumors regress and are eventually eliminated. This is not seen in the pmel-1 and Fc-IL-2 mice, as their signals strengthen with increasing tumor burden (FIG. 18). These results collectively show enhanced proliferation and survival of transferred cells only when coupled with Fc-IL-2 treatment. An increase in bioluminescence can be seen in the measurements taken following Fc-IL-2 injection, suggesting a direct link between Fc-IL-2 and T cell survival during ACT.

### Example 11

### Antigen Response for ACT Combination Therapy

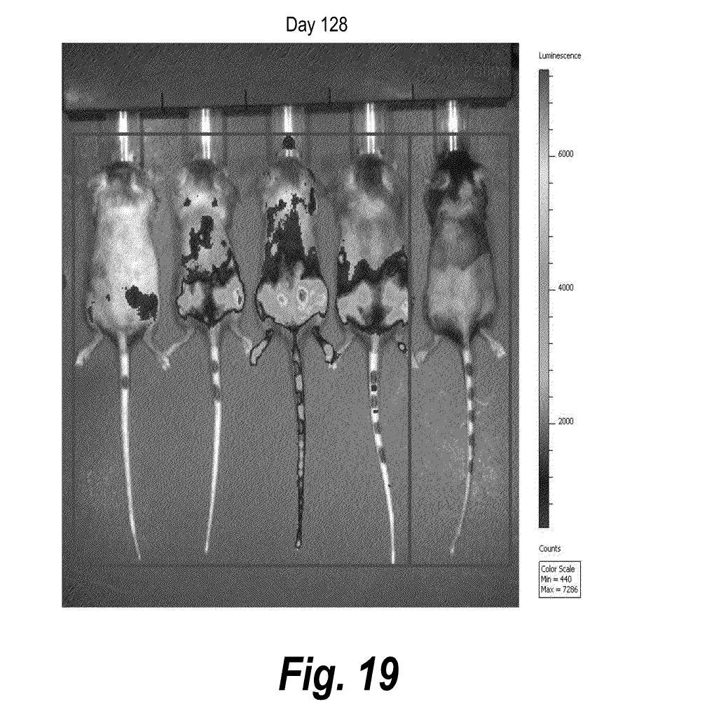

Mice undergoing ACT combination therapy (i.e., TA99, Fc-IL-2, and pmel-1), as described in Example 10, were followed for 128 days. The four surviving ACT combination treated mice and the single surviving combination treated mouse (as a negative control) were analyzed for the long-term persistence of the adoptively transferred cells. As shown in FIG. 19, the transferred pmel-1 cells persisted in mice and is a likely source of the immunological memory that the mice have against B16F10 tumors, as demonstrated by their ability to reject secondary tumors. That is, mice challenged after 60-90 days with an injection of B16-F10 cells, but given no additional therapy, do not form tumors. This is indicative of immunological memory in that the existing immune cells have recognized and eliminated a previously encountered antigen (in this case, the tumor cells) upon re-exposure.

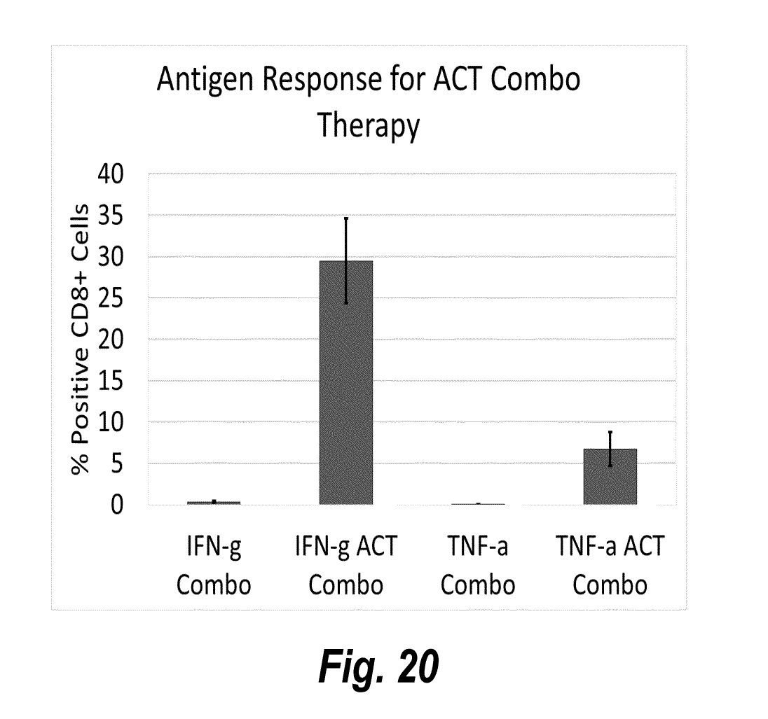

These mice were further assessed for the ability to react to an antigen (hgp-100 peptide). Intracellular cytokine staining was performed to measure antigen reactive CD8 T cells expressing IFN-γ and TNF-α. Blood samples were drawn from mice and red blood cells were lysed with ammonium-chloride-potassium buffer. The peripheral blood mononuclear cells were then resuspended in media containing hgp 100 peptide (GenScript). Brefeldin A was added after 2 hours. Four hours later, cells were harvested, fixed, permeabilized and stained for CD8, IFN-γ, and TNF-α. Cells were analyzed on a flow cytometry and gated for CD8 positive cells. The percentage of IFN-γ and TNF-α positive cells was then measured. As shown in FIG. 20, approximately 30% of the circulating T cells were antigen reactive (CD8+) after 128 days, indicating lasting tumor regression.

### Example 12

### CART Combination Therapy

Chimeric antigen receptors (CAR) are used to direct autologous tumor infiltrating lymphocytes to a specific cell target to minimize tumor burden. CD 19 is expressed by most B-cell leukemias and lymphomas and has been used in clinical trials as an effective target for CAR monotherapy. To assess whether a combination of CAR therapy with extended-PK-IL-2 and, optionally, a therapeutic antibody is more effective in treating leukemias and lymphomas the following experiment is conducted.

Peripheral blood mononuclear cells are removed from a patient and T cells are isolated by negative selection. A construct containing a single chain variable fragment (scFV) against CD19 is transfected into T cells using a lentiviral vector. The construct contains the FMC63 scFV and CD8a-CD28 transmembrane domains fused to the 4-1BB costimulatory domain and CD3z activation domain, ensuring full activation upon antigen binding (Porter et al., N Engl J Med 2011; 365:725-33; Milone et al., Molecular Therapy 2009; 17:1453-64). CAR transfected T cells are expanded in culture for 10-14 days. Prior to injection back into the patient, chemotherapy treatments are used to improve the efficacy of the engineered T cells. The autologous CAR T cells are administered to the patient with extended-PK-IL-2 and, optionally, a therapeutic antibody to increase proliferation and survival of the transferred cells and reduce tumor burden in the patient.

TABLE 3
Sequences

| SEQ | | |
|---|---|---|
| ID NO | DESCRIPTION | SEQUENCE |
| 1 | Mouse IL-2 (nucleic acid | GCACCCACTTCAAGCTCCACTTCAAGCTCTACAGCGGAAGCACAGCAGCAGCAGCAGCAGCAG |
| | sequence) | CAGCAGCAGCAGCAGCACCTGGAGCAGCTGTTGATGGACCTACAGGAGCTCCTGAGCAGGAT |
| | | GGAGAATTACAGGAACCTGAAACTCCCCAGGATGCTCACCTTCAAATTTTACTTGCCCAAGCA |
| | | GGCCACAGAATTGAAAGATCTTCAGTGCCTAGAAGATGAACTTGGACCTCTGCGGCATGTTC |
| | | TGGATTTGACTCAAAGCAAAAGCTTTCAATTGGAAGATGCTGAGAATTTCATCAGCAATATC |
| | | AGAGTAACTGTTGTAAAACTAAAGGGCTCTGACAACACATTTGAGTGCCAATTCGATGATGA |
| | | GTCAGCAACTGTGGTGGACTTTCTGAGGAGATGGATAGCCTTCTGTCAAAGCATCATCTCAA |
| | | CAAGCCCTCAA |
| 2 | Mouse IL-2 (amino acid | APTSSSTSSSTAEAQQQQQQQQQQQQHLEQLLMDLQELLSRMENYRNLKLPRMLTFKFYLPK |
| | sequence) | QATELKDLQCLEDELGPLRHVLDLTQSKSFQLEDAENFISNIRVTVVKLKGSDNTFECQFDDES |
| | | ATVVDFLRRWIAFCQSIISTSPQ |
| 3 | QQ6210 (nucleic acid | GCACCCACTTCAAGCTCCACTTCAAGCTCTACAGCGGAAGCACAACAGCAGCAGCAGCAGCAG |
| | sequence) | CAGCAGCAGCAGCAGCACCTGGAGCAGCTGTTGATGGACCTACAGGAACTCCTGAGTAGGAT |
| | | GGAGGATCACAGGAACCTGAGACTCCCCAGGATGCTCACCTTCAAATTTTACTTGCCCGAGCA |
| | | GGCCACAGAATTGGAAGATCTTCAGTGCCTAGAAGATGAACTTGAACCACTGCGGCAAGTTC |
| | | TGGATTTGACTCAAAGCAAAAGCTTTCAATTGGAAGATGCTGAGAATTTCATCAGCAATATC |
| | | AGAGTAACTGTTGTAAAACTAAAGGGCTCTGACAACACATTTGAGTGCCAATTCGACGATGA |
| | | GCCAGCAACTGTGGTGGACTTTCTGAGGAGATGGATAGCCTTCTGTCAAAGCATCATCTCAAC |
| | | AAGCCCTCAA |
| 4 | QQ6210 (amino acid | APTSSSTSSSTAEAQQQQQQQQQQQQHLEQLLMDLQELLSRMEDHRNLRLPRMLTFKFYLPE |
| | sequence) | QATELEDLQCLEDELEPLRQVLDLTQSKSFQLEDAENFISNIRVTVVKLKGSDNTFECQFDDEP |
| | | ATVVDFLRRWIAFCQSIISTSPQ |
| 5 | E76A (nucleic acid | GCACCCACTTCAAGCTCCACTTCAAGCTCTACAGCGGAAGCACAGCAGCAGCAGCAGCAGCAG |
| | sequence) | CAGCAGCAGCAGCAGCACCTGGAGCAGCTGTTGATGGACCTACAGGAGCTCCTGAGCAGGAT |
| | | GGAGAATTACAGGAACCTGAAACTCCCCAGGATGCTCACCTTCAAATTTTACTTGCCCAAGCA |
| | | GGCCACAGAATTGAAAGATCTTCAGTGCCTAGAAGATGCTCTTGGACCTCTGCGGCATGTTCT |
| | | GGATTTGACTCAAAGCAAAAGCTTTCAATTGGAAGATGCTGAGAATTTCATCAGCAATATCA |
| | | GAGTAACTGTTGTAAAACTAAAGGGCTCTGACAACACATTTGAGTGCCAATTCGATGATGAG |
| | | TCAGCAACTGTGGTGGACTTTCTGAGGAGATGGATAGCCTTCTGTCAAAGCATCATCTCAAC |
| | | AAGCCCTCAA |
| 6 | E76A (amino acid | APTSSSTSSSTAEAQQQQQQQQQQQQHLEQLLMDLQELLSRMENYRNLKLPRMLTFKFYLPK |
| | sequence) | QATELKDLQCLEDALGPLRHVLDLTQSKSFQLEDAENFISNIRVTVVKLKGSDNTFECQFDDES |
| | | ATVVDFLRRWIAFCQSIISTSPQ |
| 7 | E76G (nucleic acid | GCACCCACTTCAAGCTCCACTTCAAGCTCTACAGCGGAAGCACAGCAGCAGCAGCAGCAGCAG |
| | sequence) | CAGCAGCAGCAGCAGCACCTGGAGCAGCTGTTGATGGACCTACAGGAGCTCCTGAGCAGGAT |
| | | GGAGAATTACAGGAACCTGAAACTCCCCAGGATGCTCACCTTCAAATTTTACTTGCCCAAGCA |
| | | GGCCACAGAATTGAAAGATCTTCAGTGCCTAGAAGATGGTCTTGGACCTCTGCGGCATGTTCT |
| | | GGATTTGACTCAAAGCAAAAGCTTTCAATTGGAAGATGCTGAGAATTTCATCAGCAATATCA |
| | | GAGTAACTGTTGTAAAACTAAAGGGCTCTGACAACACATTTGAGTGCCAATTCGATGATGAG |
| | | TCAGCAACTGTGGTGGACTTTCTGAGGAGATGGATAGCCTTCTGTCAAAGCATCATCTCAAC |
| | | AAGCCCTCAA |
| 8 | E76G (amino acid | APTSSSTSSSTAEAQQQQQQQQQQQQHLEQLLMDLQELLSRMENYRNLKLPRMLTFKFYLPK |
| | sequence) | QATELKDLQCLEDGLGPLRHVLDLTQSKSFQLEDAENFISNIRVTVVKLKGSDNTFECQFDDES |
| | | ATVVDFLRRWIAFCQSIISTSPQ |
| 9 | D265A Fc/Flag (nucleic | ATGAGGGTCCCCGCTCAGCTCCTGGGGCTCCTGCTGCTCTGGCTCCCAGGTGCACGATGTGAG |
| | acid sequence) | CCCAGAGTGCCCATAACACAGAACCCCTGTCCTCCACTCAAAGAGTGTCCCCCATGCGCAGCT |
| | (C-terminal flag tag | CCAGACCTCTTGGGTGGACCATCCGTCTTCATCTTCCCTCCAAAGATCAAGGATGTACTCATG |
| | is underlined) | ATCTCCCTGAGCCCCATGGTCACATGTGTGGTGGTGGCCGTGAGCGAGGATGACCCAGACGTC |
| | | CAGATCAGCTGGTTTGTGAACAACGTGGAAGTACACACAGCTCAGACACAAACCCATAGAGA |
| | | GGATTACAACAGTACTCTCCGGGTGGTCAGTGCCCTCCCCATCCAGCACCAGGACTGGATGAG |
| | | TGGCAAGGAGTTCAAATGCAAGGTCAACAACAGAGCCCTCCCATCCCCCATCGAGAAAACCAT |
| | | CTCAAAACCCAGAGGGCCAGTAAGAGCTCCACAGGTATATGTCTTGCCTCCACCAGCAGAAGA |
| | | GATGACTAAGAAAGAGTTCAGTCTGACCTGCATGATCACAGGCTTCTTACCTGCCGAAATTGC |
| | | TGTGGACTGGACCAGCAATGGGCGTACAGAGCAAAACTACAAGAACACCGCAACAGTCCTGG |
| | | ACTCTGATGGTTCTTACTTCATGTACAGCAAGCTCAGAGTACAAAAGAGCACTTGGGAAAGA |
| | | GGAAGTCTTTTCGCCTGCTCAGTGGTCCACGAGGGTCTGCACAATCACCTTACGACTAAGACC |
| | | ATCTCCCGGTCTCTGGGTAAAGGTGGCGGATCTGACTACAAGGACGACGATGACAAGTGATA |
| | | A |
| 10 | D265A Fc/Flag (amino | MRVPAQLLGLLLLWLPGARCEPRVPITQNPCPPLKECPPCAAPDLLGGPSVFIFPPKIKDVLMIS |
| | acid sequence) | LSPMVTCVVVAVSEDDPDVQISWFVNNVEVHTAQTQTHREDYNSTLRVVSALPIQHQDWMS |
| | (C-terminal flag tag | GKEFKCKVNNRALPSPIEKTISKPRGPVRAPQVYVLPPPAEEMTKKEFSLTCMITGFLPAEIAV |
| | is underlined) | DWTSNGRTEQNYKNTATVLDSDGSYFMYSKLRVQKSTWERGSLFACSVVHEGLHNHLTTKTI |
| | | SRSLGKGGGSDYKDDDDK |
| 11 | D265A Fc/wt mIL-2 | ATGAGGGTCCCCGCTCAGCTCCTGGGGCTCCTGCTGCTCTGGCTCCCAGGTGCACGATGTGAG |
| | (nucleic acid sequence) | CCCAGAGTGCCCATAACACAGAACCCCTGTCCTCCACTCAAAGAGTGTCCCCCATGCGCAGCT |
| | (C-terminal 6× his tag | CCAGACCTCTTGGGTGGACCATCCGTCTTCATCTTCCCTCCAAAGATCAAGGATGTACTCATG |
| | is underlined) | ATCTCCCTGAGCCCCATGGTCACATGTGTGGTGGTGGCCGTGAGCGAGGATGACCCAGACGTC |
| | | CAGATCAGCTGGTTTGTGAACAACGTGGAAGTACACACAGCTCAGACACAAACCCATAGAGA |
| | | GGATTACAACAGTACTCTCCGGGTGGTCAGTGCCCTCCCCATCCAGCACCAGGACTGGATGAG |
| | | TGGCAAGGAGTTCAAATGCAAGGTCAACAACAGAGCCCTCCCATCCCCCATCGAGAAAACCAT |
| | | CTCAAAACCCAGAGGGCCAGTAAGAGCTCCACAGGTATATGTCTTGCCTCCACCAGCAGAAGA |
| | | GATGACTAAGAAAGAGTTCAGTCTGACCTGCATGATCACAGGCTTCTTACCTGCCGAAATTGC |
| | | TGTGGACTGGACCAGCAATGGGCGTACAGAGCAAAACTACAAGAACACCGCAACAGTCCTGG |
| | | ACTCTGATGGTTCTTACTTCATGTACAGCAAGCTCAGAGTACAAAAGAGCACTTGGGAAAGA |
| | | GGAAGTCTTTTCGCCTGCTCAGTGGTCCACGAGGGTCTGCACAATCACCTTACGACTAAGACC |
| | | ATCTCCCGGTCTCTGGGTAAAGGAGGGGGCTCCGCACCCACTTCAAGCTCCACTTCAAGCTCT |
| | | ACAGCGGAAGCACAGCAGCAGCAGCAGCAGCAGCAGCAGCAGCAGCAGCACCTGGAGCAGCT |
| | | GTTGATGGACCTACAGGAGCTCCTGAGCAGGATGGAGAATTACAGGAACCTGAAACTCCCCA |
| | | GGATGCTCACCTTCAAATTTTACTTGCCCAAGCAGGCCACAGAATTGAAAGATCTTCAGTGCC |
| | | TAGAAGATGAACTTGGACCTCTGCGGCATGTTCTGGATTTGACTCAAAGCAAAAGCTTTCAA |
| | | TTGGAAGATGCTGAGAATTTCATCAGCAATATCAGAGTAACTGTTGTAAAACTAAAGGGCTC |
| | | TGACAACACATTTGAGTGCCAATTCGATGATGAGTCAGCAACTGTGGTGGACTTTCTGAGGA |
| | | GATGGATAGCCTTCTGTCAAAGCATCATCTCAACAAGCCCTCAACACCATCACCACCATCACT |
| | | GATAA |
| 12 | D265A Fc/wt mIL-2 | MRVPAQLLGLLLLWLPGARCEPRVPITQNPCPPLKECPPCAAPDLLGGPSVFIFPPKIKDVLMIS |
| | (amino acid sequence) | LSPMVTCVVVAVSEDDPDVQISWFVNNVEVHTAQTQTHREDYNSTLRVVSALPIQHQDWMS |
| | (C-terminal 6× his tag | GKEFKCKVNNRALPSPIEKTISKPRGPVRAPQVYVLPPPAEEMTKKEFSLTCMITGFLPAEIAV |
| | is underlined) | DWTSNGRTEQNYKNTATVLDSDGSYFMYSKLRVQKSTWERGSLFACSVVHEGLHNHLTTKTI |
| | | SRSLGKGGGSAPTSSSTSSSTAEAQQQQQQQQQQQQHLEQLLMDLQELLSRMENYRNLKLPR |
| | | MLTFKFYLPKQATELKDLQCLEDELGPLRHVLDLTQSKSFQLEDAENFISNIRVTVVKLKGSDN |
| | | TFECQFDDESATWDFLRRWIAFCQSIISTSPQHHHHHH** |
| 13 | D265A Fc/QQ6210 | ATGAGGGTCCCCGCTCAGCTCCTGGGGCTCCTGCTGCTCTGGCTCCCAGGTGCACGATGTGAG |
| | (nucleic acid sequence) | CCCAGAGTGCCCATAACACAGAACCCCTGTCCTCCACTCAAAGAGTGTCCCCCATGCGCAGCT |
| | (C-terminal 6× his tag | CCAGACCTCTTGGGTGGACCATCCGTCTTCATCTTCCCTCCAAAGATCAAGGATGTACTCATG |
| | is underlined) | ATCTCCCTGAGCCCCATGGTCACATGTGTGGTGGTGGCCGTGAGCGAGGATGACCCAGACGTC |
| | | CAGATCAGCTGGTTTGTGAACAACGTGGAAGTACACACAGCTCAGACACAAACCCATAGAGA |
| | | GGATTACAACAGTACTCTCCGGGTGGTCAGTGCCCTCCCCATCCAGCACCAGGACTGGATGAG |
| | | TGGCAAGGAGTTCAAATGCAAGGTCAACAACAGAGCCCTCCCATCCCCCATCGAGAAAACCAT |
| | | CTCAAAACCCAGAGGGCCAGTAAGAGCTCCACAGGTATATGTCTTGCCTCCACCAGCAGAAGA |
| | | GATGACTAAGAAAGAGTTCAGTCTGACCTGCATGATCACAGGCTTCTTACCTGCCGAAATTGC |
| | | TGTGGACTGGACCAGCAATGGGCGTACAGAGCAAAACTACAAGAACACCGCAACAGTCCTGG |
| | | ACTCTGATGGTTCTTACTTCATGTACAGCAAGCTCAGAGTACAAAAGAGCACTTGGGAAAGA |
| | | GGAAGTCTTTTCGCCTGCTCAGTGGTCCACGAGGGTCTGCACAATCACCTTACGACTAAGACC |
| | | ATCTCCCGGTCTCTGGGTAAAGGAGGGGGCTCCGCACCCACTTCAAGCTCCACTTCAAGCTCT |
| | | ACAGCGGAAGCACAACAGCAGCAGCAGCAGCAGCAGCAGCAGCAGCAGCACCTGGAGCAGCT |
| | | GTTGATGGACCTACAGGAACTCCTGAGTAGGATGGAGGATCACAGGAACCTGAGACTCCCCA |
| | | GGATGCTCACCTTCAAATTTTACTTGCCCGAGCAGGCCACAGAATTGGAAGATCTTCAGTGCC |
| | | TAGAAGATGAACTTGAACCACTGCGGCAAGTTCTGGATTTGACTCAAAGCAAAAGCTTTCAA |
| | | TTGGAAGATGCTGAGAATTTCATCAGCAATATCAGAGTAACTGTTGTAAAACTAAAGGGCTC |
| | | TGACAACACATTTGAGTGCCAATTCGACGATGAGCCAGCAACTGTGGTGGACTTTCTGAGGA |
| | | GATGGATAGCCTTCTGTCAAAGCATCATCTCAACAAGCCCTCAACACCATCACCACCATCACT |
| | | GATAA |
| 14 | D265A Fc /QQ6210 | MRVPAQLLGLLLLWLPGARCEPRVPITQNPCPPLKECPPCAAPDLLGGPSVFIFPPKIKDVLMIS |
| | (amino acid sequence) | LSPMVTCVVVAVSEDDPDVQISWFVNNVEVHTAQTQTHREDYNSTLRVVSALPIQHQDWMS |
| | (C-terminal 6× his tag | GKEFKCKVNNRALPSPIEKTISKPRGPVRAPQVYVLPPPAEEMTKKEFSLTCMITGFLPAEIAV |
| | is underlined) | DWTSNGRTEQNYKNTATVLDSDGSYFMYSKLRVQKSTWERGSLFACSVVHEGLHNHLTTKTI |
| | | SRSLGKGGGSAPTSSSTSSSTAEAQQQQQQQQQQQQHLEQLLMDLQELLSRMEDHRNLRLPR |
| | | MLTFKFYLPEQATELEDLQCLEDELEPLRQVLDLTQSKSFQLEDAENFISNIRVTVVKLKGSDN |
| | | TFECQFDDEPATWDFLRRWIAFCQSIISTSPQHHHHHH |
| 15 | D265A Fc/E76A (nucleic | ATGAGGGTCCCCGCTCAGCTCCTGGGGCTCCTGCTGCTCTGGCTCCCAGGTGCACGATGTGAG |
| | acid sequence) | CCCAGAGTGCCCATAACACAGAACCCCTGTCCTCCACTCAAAGAGTGTCCCCCATGCGCAGCT |
| | (C-terminal 6× his tag | CCAGACCTCTTGGGTGGACCATCCGTCTTCATCTTCCCTCCAAAGATCAAGGATGTACTCATG |
| | is underlined) | ATCTCCCTGAGCCCCATGGTCACATGTGTGGTGGTGGCCGTGAGCGAGGATGACCCAGACGTC |
| | | CAGATCAGCTGGTTTGTGAACAACGTGGAAGTACACACAGCTCAGACACAAACCCATAGAGA |
| | | GGATTACAACAGTACTCTCCGGGTGGTCAGTGCCCTCCCCATCCAGCACCAGGACTGGATGAG |
| | | TGGCAAGGAGTTCAAATGCAAGGTCAACAACAGAGCCCTCCCATCCCCCATCGAGAAAACCAT |
| | | CTCAAAACCCAGAGGGCCAGTAAGAGCTCCACAGGTATATGTCTTGCCTCCACCAGCAGAAGA |
| | | GATGACTAAGAAAGAGTTCAGTCTGACCTGCATGATCACAGGCTTCTTACCTGCCGAAATTGC |
| | | TGTGGACTGGACCAGCAATGGGCGTACAGAGCAAAACTACAAGAACACCGCAACAGTCCTGG |
| | | ACTCTGATGGTTCTTACTTCATGTACAGCAAGCTCAGAGTACAAAAGAGCACTTGGGAAAGA |
| | | GGAAGTCTTTTCGCCTGCTCAGTGGTCCACGAGGGTCTGCACAATCACCTTACGACTAAGACC |
| | | ATCTCCCGGTCTCTGGGTAAAGGAGGGGGCTCCGCACCCACTTCAAGCTCCACTTCAAGCTCT |
| | | ACAGCGGAAGCACAGCAGCAGCAGCAGCAGCAGCAGCAGCAGCAGCAGCACCTGGAGCAGCT |
| | | GTTGATGGACCTACAGGAGCTCCTGAGCAGGATGGAGAATTACAGGAACCTGAAACTCCCCA |
| | | GGATGCTCACCTTCAAATTTTACTTGCCCAAGCAGGCCACAGAATTGAAAGATCTTCAGTGCC |
| | | TAGAAGATGCTCTTGGACCTCTGCGGCATGTTCTGGATTTGACTCAAAGCAAAAGCTTTCAA |
| | | TTGGAAGATGCTGAGAATTTCATCAGCAATATCAGAGTAACTGTTGTAAAACTAAAGGGCTC |
| | | TGACAACACATTTGAGTGCCAATTCGATGATGAGTCAGCAACTGTGGTGGACTTTCTGAGGA |
| | | GATGGATAGCCTTCTGTCAAAGCATCATCTCAACAAGCCCTCAACACCATCACCACCATCACT |
| | | GATAA |
| 16 | D265A Fc/E76A (amino | MRVPAQLLGLLLLWLPGARCEPRVPITQNPCPPLKECPPCAAPDLLGGPSVFIFPPKIKDVLMIS |
| | acid sequence) | LSPMVTCVVVAVSEDDPDVQISWFVNNVEVHTAQTQTHREDYNSTLRVVSALPIQHQDWMS |
| | (C-terminal 6× his tag | GKEFKCKVNNRALPSPIEKTISKPRGPVRAPQVYVLPPPAEEMTKKEFSLTCMITGFLPAEIAV |
| | is underlined) | DWTSNGRTEQNYKNTATVLDSDGSYFMYSKLRVQKSTWERGSLFACSVVHEGLHNHLTTKTI |
| | | SRSLGKGGGSAPTSSSTSSSTAEAQQQQQQQQQQQQHLEQLLMDLQELLSRMENYRNLKLPR |
| | | MLTFKFYLPKQATELKDLQCLEDALGPLRHVLDLTQSKSFQLEDAENFISNIRVTVVKLKGSDN |
| | | TFECQFDDESATWDFLRRWIAFCQSIISTSPQHHHHHH |
| 17 | D265A Fc/E76G (nucleic | ATGAGGGTCCCCGCTCAGCTCCTGGGGCTCCTGCTGCTCTGGCTCCCAGGTGCACGATGTGAG |
| | acid sequence) | CCCAGAGTGCCCATAACACAGAACCCCTGTCCTCCACTCAAAGAGTGTCCCCCATGCGCAGCT |
| | (C-terminal 6× his tag | CCAGACCTCTTGGGTGGACCATCCGTCTTCATCTTCCCTCCAAAGATCAAGGATGTACTCATG |
| | is underlined) | ATCTCCCTGAGCCCCATGGTCACATGTGTGGTGGTGGCCGTGAGCGAGGATGACCCAGACGTC |
| | | CAGATCAGCTGGTTTGTGAACAACGTGGAAGTACACACAGCTCAGACACAAACCCATAGAGA |
| | | GGATTACAACAGTACTCTCCGGGTGGTCAGTGCCCTCCCCATCCAGCACCAGGACTGGATGAG |
| | | TGGCAAGGAGTTCAAATGCAAGGTCAACAACAGAGCCCTCCCATCCCCCATCGAGAAAACCAT |
| | | CTCAAAACCCAGAGGGCCAGTAAGAGCTCCACAGGTATATGTCTTGCCTCCACCAGCAGAAGA |
| | | GATGACTAAGAAAGAGTTCAGTCTGACCTGCATGATCACAGGCTTCTTACCTGCCGAAATTGC |
| | | TGTGGACTGGACCAGCAATGGGCGTACAGAGCAAAACTACAAGAACACCGCAACAGTCCTGG |
| | | ACTCTGATGGTTCTTACTTCATGTACAGCAAGCTCAGAGTACAAAAGAGCACTTGGGAAAGA |
| | | GGAAGTCTTTTCGCCTGCTCAGTGGTCCACGAGGGTCTGCACAATCACCTTACGACTAAGACC |
| | | ATCTCCCGGTCTCTGGGTAAAGGAGGGGGCTCCGCACCCACTTCAAGCTCCACTTCAAGCTCT |
| | | ACAGCGGAAGCACAGCAGCAGCAGCAGCAGCAGCAGCAGCAGCAGCAGCACCTGGAGCAGCT |
| | | GTTGATGGACCTACAGGAGCTCCTGAGCAGGATGGAGAATTACAGGAACCTGAAACTCCCCA |
| | | GGATGCTCACCTTCAAATTTTACTTGCCCAAGCAGGCCACAGAATTGAAAGATCTTCAGTGCC |
| | | TAGAAGATGGTCTTGGACCTCTGCGGCATGTTCTGGATTTGACTCAAAGCAAAAGCTTTCAA |
| | | TTGGAAGATGCTGAGAATTTCATCAGCAATATCAGAGTAACTGTTGTAAAACTAAAGGGCTC |
| | | TGACAACACATTTGAGTGCCAATTCGATGATGAGTCAGCAACTGTGGTGGACTTTCTGAGGA |
| | | GATGGATAGCCTTCTGTCAAAGCATCATCTCAACAAGCCCTCAACACCATCACCACCATCACT |
| | | GATAA |
| 18 | D265A Fc/E76G (amino | MRVPAQLLGLLLLWLPGARCEPRVPITQNPCPPLKECPPCAAPDLLGGPSVFIFPPKIKDVLMIS |
| | acid sequence) | LSPMVTCVVVAVSEDDPDVQISWFVNNVEVHTAQTQTHREDYNSTLRVVSALPIQHQDWMS |
| | (C-terminal 6× his tag | GKEFKCKVNNRALPSPIEKTISKPRGPVRAPQVYVLPPPAEEMTKKEFSLTCMITGFLPAEIAV |
| | is underlined) | DWTSNGRTEQNYKNTATVLDSDGSYFMYSKLRVQKSTWERGSLFACSVVHEGLHNHLTTKTI |
| | | SRSLGKGGGSAPTSSSTSSSTAEAQQQQQQQQQQQQHLEQLLMDLQELLSRMENYRNLKLPR |
| | | MLTFKFYLPKQATELKDLQCLEDGLGPLRHVLDLTQSKSFQLEDAENFISNIRVTVVKLKGSDN |
| | | TFECQFDDESATWDFLRRWIAFCQSIISTSPQHHHHHH |
| 19 | mIL-2 QQ 6.2-4 (nucleic | GCACCCACCTCAAGCTCCACTTCAAGCTCTACAGCGGAAGCACAACAGCAGCAGCAGCAGCAG |
| | acid sequence) | CAGCAGCAGCAGCAGCACCTGGAGCAGCTGTTGATGGACCTACAGGAGCTCCTGAGCAGGAT |
| | | GGAGGATTCCAGGAACCTGAGACTCCCCAGGATGCTCACCTTCAAATTTTACTTGCCCAAGCA |
| | | GGCCACAGAATTGGAAGATCTTCAGTGCCTAGAAGATGAACTTGAACCTCTGCGGCAAGTTC |
| | | TGGATTTGACTCAAAGCAAAAGCTTTCAATTGGAAGATGCTGAGAATTTCATCAGCAATATC |
| | | AGAGTAACTGTTGTAAAACTAAAGGGCTCTGACAACACATTTGAGTGCCAATTCGATGATGA |
| | | GCCAGCAACTGTGGTGGGCTTTCTGAGGAGATGGATAGCCTTCTGTCAAAGCATCATCTCAAC |
| | | GAGCCCTCAA |
| 20 | mIL-2 QQ 6.2-4 (amino | APTSSSTSSSTAEAQQQQQQQQQQQQHLEQLLMDLQELLSRMEDSRNLRLPRMLTFKFYLPK |
| | acid sequence) | QATELEDLQCLEDELEPLRQVLDLTQSKSFQLEDAENFISNIRVTVVKLKGSDNTFECQFDDEP |
| | | ATVVGFLRRWIAFCQSIISTSPQ |
| 21 | mIL-2 QQ 6.2-8 (nucleic | GCACCCACCTCAAGCTCCACTTCAAGCTCTACAGCGGAAGCACAACAGCAGCAGCAGCAGCAG |
| | acid sequence) | CAGCACCTGGAGCAGCTGTTGATGGACCTACAGGAGCTCCTGAGTAGGATGGAGGATCACAG |
| | | GAACCTGAGACTCCCCAGGATGCTCACCTTCAAATTTTACTTGCCCAAGCAGGCCACAGAATT |
| | | GGAAGATCTTCAGTGCCTAGAAGATGAACTTGAACCTCTGCGGCAAGTTCTGGATTTGACTC |
| | | AAAGCAAAAGCTTTCAATTGGAAGATGCTGAGAATTTCATCAGCAATATCAGAGTAACTGTT |
| | | GTAAAACTAAAGGGCTCTGACAACACATTTGAGTGCCAATTCGATGATGAGCCAGCAACTGT |
| | | GGTGGACTTTCTGAGGAGATGGATAGCCTTCTGTCAAAGCATCATCTCAACAAGCCCTCGA |
| 22 | mIL-2 QQ 6.2-8 (amino | APTSSSTSSSTAEAQQQQQQQQHLEQLLMDLQELLSRMEDHRNLRLPRMLTFKFYLPKQATE |
| | acid sequence) | LEDLQCLEDELEPLRQVLDLTQSKSFQLEDAENFISNIRVTVVKLKGSDNTFECQFDDEPATVV |
| | | DFLRRWIAFCQSIISTSPR |
| 23 | mIL-2 QQ 6.2-10 (nucleic | GCACCCACCTCAAGCTCCACTTCAAGCTCTACAGCGGAAGCACAACAGCAGCAGCAGCAGCAG |
| | acid sequence) | CAGCAGCAGCAGCAGCACCTGGAGCAGCTGTTGATGGACCTACAGGAACTCCTGAGTAGGAT |
| | | GGAGGATCACAGGAACCTGAGACTCCCCAGGATGCTCACCTTCAAATTTTACTTGCCCGAGCA |
| | | GGCCACAGAATTGGAAGATCTTCAGTGCCTAGAAGATGAACTTGAACCACTGCGGCAAGTTC |
| | | TGGATTTGACTCAAAGCAAAAGCTTTCAATTGGAAGATGCTGAGAATTTCATCAGCAATATC |
| | | AGAGTAACTGTTGTAAAACTAAAGGGCTCTGACAACACATTTGAGTGCCAATTCGACGATGA |
| | | GCCAGCAACTGTGGTGGACTTTCTGAGGAGATGGATAGCCTTCTGTCAAAGCATCATCTCAAC |
| | | AAGCCCTCAG |
| 24 | mIL-2 QQ 6.2-10 (amino | APTSSSTSSSTAEAQQQQQQQQQQQQHLEQLLMDLQELLSRMEDHRNLRLPRMLTFKFYLPE |
| | acid sequence) | QATELEDLQCLEDELEPLRQVLDLTQSKSFQLEDAENFISNIRVTVVKLKGSDNTFECQFDDEP |
| | | ATVVDFLRRWIAFCQSIISTSPQ |
| 25 | mIL-2 QQ 6.2-11 (nucleic | GCACCCACCTCAAGCTCCACTTCAAGCTCTACAGCGGAAGCACAACAGCAGCAGCAGCAGCAG |
| | acid sequence) | CAGCAGCACCTGGAGCAGCTGTTGATGGACCTACAGGAGCTCCTGAGCAGGATGGAGGATTC |
| | | CAGGAACCTGAGACTCCCCAGAATGCTCACCTTCAAATTTTACTTGCCCGAGCAGGCCACAGA |
| | | ATTGAAAGATCTCCAGTGCCTAGAAGATGAACTTGAACCTCTGCGGCAAGTTCTGGATTTGA |
| | | CTCAAAGCAAAAGCTTTCAATTGGAAGATGCTGAGAATTTCATCAGCAATATCAGAGTAACT |
| | | GTTGTAAAACTAAAGGGCTCTGACAACACATTTGAGTGCCAATTCGACGATGAGCCAGCAAC |
| | | TGTGGTGGACTTTCTGAGGAGATGGATAGCCTTCTGTCAAAGCATCATCTCAACAAGCCCTCA |
| | | G |
| 26 | mIL-2 QQ 6.2-11 (amino | APTSSSTSSSTAEAQQQQQQQQQHLEQLLMDLQELLSRMEDSRNLRLPRMLTFKFYLPEQATE |
| | acid sequence) | LKDLQCLEDELEPLRQVLDLTQSKSFQLEDAENFISNIRVTVVKLKGSDNTFECQFDDEPATVV |
| | | DFLRRWIAFCQSIISTSPQ |
| 27 | mIL-2 QQ 6.2-13 (nucleic | GCACCCACCTCAAGCTCCACTTCAAGCTCTACAGCGGAAGCACAACAGCAGCAGCAGCAGCAG |
| | acid sequence) | CAGCAGCAGCAGCAGCACCTGGAGCAGCTGTTGATGGACCTACAGGAGCTCCTGAGTAGGAT |
| | | GGAGGATCACAGGAACCTGAGACTCCCCAGGATGCTCACCTTCAAATTTTACTTGCCCGAGCA |
| | | GGCCACAGAATTGAAAGATCTCCAGTGCCTAGAAGATGAACTTGAACCTCTGCGGCAGGTTC |
| | | TGGATTTGACTCAAAGCAAAAGCTTTCAATTGGAAGATGCTGAGAATTTCATCAGCAATATC |
| | | AGAGTAACTGTTGTAAAACTAAAGGGCTCTGACAACACATTTGAGTGCCAATTCGATGATGA |
| | | GCCAGCAACTGTGGTGGACTTTCTGAGGAGATGGATAGCCTTCTGTCAAAGCATCATCTCAAC |
| | | AAGCCCTCAG |
| 28 | mIL-2 QQ 6.2-13 (amino | APTSSSTSSSTAEAQQQQQQQQQQQQHLEQLLMDLQELLSRMEDHRNLRLPRMLTFKFYLPE |
| | acid sequence) | QATELKDLQCLEDELEPLRQVLDLTQSKSFQLEDAENFISNIRVTVVKLKGSDNTFECQFDDEP |
| | | ATVVDFLRRWIAFCQSIISTSPQ |
| 29 | Full length human IL-2 | ATGTACAGGATGCAACTCCTGTCTTGCATTGCACTAAGTCTTGCACTTGTCACAAACAGTGCA |
| | (nucleic acid sequence) | CCTACTTCAAGTTCTACAAAGAAAACACAGCTACAACTGGAGCATTTACTGCTGGATTTACA |
| | | GATGATTTTGAATGGAATTAATAATTACAAGAATCCCAAACTCACCAGGATGCTCACATTTA |
| | | AGTTTTACATGCCCAAGAAGGCCACAGAACTGAAACATCTTCAGTGTCTAGAAGAAGAACTC |
| | | AAACCTCTGGAGGAAGTGCTAAATTTAGCTCAAAGCAAAAACTTTCACTTAAGACCCAGGGA |
| | | CTTAATCAGCAATATCAACGTAATAGTTCTGGAACTAAAGGGATCTGAAACAACATTCATGT |
| | | GTAATATGCTGATGAGACAGCAACCATTGTAGAATTTCTGAACAGATGGATTACCTTTTGTC |
| | | AAAGCATCATCTCAACACTGACTTGA |
| 30 | Full length human IL-2 | MYRMQLLSCIALSLALVTNSAPTSSSTKKTQLQLEHLLLDLQMILNGINNYKNPKLTR |
| | (amino acid sequence) | MLTEKEYMPKKATELKIALQCLEEELKPLEEVLNLAQSKNEHLRPRDLISNINVIVLEL |
| | | KGSETTFMCEYADETATIVEFLNRWITFCQSIISTLT |
| 31 | Human IL-2 without | GCACCTACTTCAAGTTCTACAAAGAAAACACAGCTACAACTGGAGCATTTACTGCTGGATTT |
| | signal peptide | ACAGATGATTTTGAATGGAATTAATAATTACAAGAATCCCAAACTCACCAGGATGCTCACAT |
| | (nucleic acid | TTAAGTTTTACATGCCCAAGAAGGCCACAGAACTGAAACATCTTCAGTGTCTAGAAGAAGAA |
| | sequence) | CTCAAACCTCTGGAGGAAGTGCTAAATTTAGCTCAAAGCAAAAACTTTCACTTAAGACCCAG |
| | | GGACTTAATCAGCAATATCAACGTAATAGTTCTGGAACTAAAGGGATCTGAAACAACATTCA |
| | | TGTGTAATATGCTGATGAGACAGCAACCATTGTAGAATTTCTGAACAGATGGATTACCTTTT |
| | | GTCAAAGCATCATCTCAACACTGACTTGA |
| 32 | Human IL-2 without | APTSSSTKKTQLQLEHLLLDLQMILNGINNYKNPKLTRMLTFKFYMPKKATELKHLQCLEEEL |
| | signal peptide (amino | KPLEEVLNLAQSKNFHLRPRDLISNINVIVLELKGSETTFMCEYADETATIVEFLNRWITFCQSII |
| | acid sequence) | STLT |
| 33 | Human IgG1 constant | ASTKGPSVFPLAPSSKSTSGGTAALGCLVKDYFPEPVTVSWNSGALTSGVHTFPAVLQSSGLYSL |
| | region (amino acid | SSVVTVPSSSLGTQTYICNVNHKPSNTKVDKKVEPKSCDKTHTCPPCPAPELLGGPSVFLFPPK |
| | sequence) | PKDTLMISRTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRDELTKNQVSLTCLVKG |
| | | FYPSDIAVEWESNGQPENNYKTTPPVLDSDGSFFLYSKLTVDKSRWQQGNVFSCSVMHEALH |
| | | NHYTQKSLSLSPGK |
| 34 | Human IgG1 Fc domain | EPKSCDKTHTCPPCPAPELLGGPSVFLFPPKPKDTLMISRTPEVTCVVVDVSHEDPEVKFNWY |
| | (amino acid sequence) | VDGVEVHNAKTKPREEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 35 | Human serum albumin | MKWVTFISLLFLFSSAYSRGVFRRDAHKSEVAHRFKDLGEENFKALVLIAFAQYLQQCPFEDH |
| | (amino acid sequence) | VKLVNEVTEFAKTCVADESAENCDKSLHTLFGDKLCTVATLRETYGEMADCCAKQEPERNEC |
| | | FLQHKDDNPNLPRLVRPEVDVMCTAFHDNEETFLKKYLYEIARRHPYFYAPELLFFAKRYKAA |
| | | FTECCQAADKAACLLPKLDELRDEGKASSAKQRLKCASLQKFGERAFKAWAVARLSQRFPKA |
| | | EFAEVSKLVTDLTKVHTECCHGDLLECADDRADLAKYICENQDSISSKLKECCEKPLLEKSHCIA |
| | | EVENDEMPADLPSLAADFVESKDVCKNYAEAKDVFLGMFLYEYARRHPDYSVVLLLRLAKTY |
| | | ETTLEKCCAAADPHECYAKVFDEFKPLVEEPQNLIKQNCELFEQLGEYKFQNALLVRYTKKVP |
| | | QVSTPTLVEVSRNLGKVGSKCCKHPEAKRMPCAEDYLSVVLNQLCVLHEKTPVSDRVTKCCTE |
| | | SLVNRRPCFSALEVDETYVPKEFNAETFTFHADICTLSEKERQIKKQTALVELVKHKPKATKEQ |
| | | LKAVMDDFAAFVEKCCKADDKETCFAEEGKKLVAASQAALGL |
| 36 | Mature HSA (amino acid | DAHKSEVAHRFKDLGEENFKALVLIAFAQYLQQCPFEDHVKLVNEVTEFAKTCVADESAENCD |
| | sequence) | KSLHTLFGDKLCTVATLRETYGEMADCCAKQEPERNECFLQHKDDNPNLPRLVRPEVDVMCT |
| | | AFHDNEETFLKKYLYEIARRHPYFYAPELLFFAKRYKAAFTECCQAADKAACLLPKLDELRDE |
| | | GKASSAKQRLKCASLQKFGERAFKAWAVARLSQRFPKAEFAEVSKLVTDLTKVHTECCHGDLL |
| | | ECADDRADLAKYICENQDSISSKLKECCEKPLLEKSHCIAEVENDEMPADLPSLAADFVESKDV |
| | | CKNYAEAKDVFLGMFLYEYARRHPDYSWLLLRLAKTYETTLEKCCAAADPHECYAKVFDEFK |
| | | PLVEEPQNLIKQNCELFEQLGEYKFQNALLVRYTKKVPQVSTPTLVEVSRNLGKVGSKCCKHPE |
| | | AKRMPCAEDYLSVVLNQLCVLHEKTPVSDRVTKCCTESLVNRRPCFSALEVDETYVPKEFNAE |
| | | TFTFHADICTLSEKERQIKKQTALVELVKHKPKATKEQLKAVMDDFAAFVEKCCKADDKETCF |
| | | AEEGKKLVAASQAALGL |
| 37 | Mature HSA (nucleic acid | GATGCTCACAAAAGCGAAGTCGCACACAGGTTCAAAGATCTGGGGGAGGAAAACTTTAAGGC |
| | sequence) | TCTGGTGCTGATTGCATTCGCCCAGTACCTGCAGCAGTGCCCCTTTGAGGACCACGTGAAACT |
| | | GGTCAACGAAGTGACTGAGTTCGCCAAGACCTGCGTGGCCGACGAATCTGCTGAGAATTGTG |
| | | ATAAAAGTCTGCATACTCTGTTTGGGGATAAGCTGTGTACAGTGGCCACTCTGCGAGAAACC |
| | | TATGGAGAGATGGCAGACTGCTGTGCCAAACAGGAACCCGAGCGGAACGAATGCTTCCTGCA |
| | | GCATAAGGACGATAACCCCAATCTGCCTCGCCTGGTGCGACCTGAGGTGGACGTCATGTGTAC |
| | | AGCCTTCCACGATAATGAGGAAACTTTTCTGAAGAAATACCTGTACGAAATCGCTCGGAGAC |
| | | ATCCTTACTTTTATGCACCAGAGCTGCTGTTCTTTGCCAAACGCTACAAGGCCGCTTTCACCG |
| | | AGTGCTGTCAGGCAGCCGATAAAGCTGCATGCCTGCTGCCTAAGCTGGACGAACTGAGGGAT |
| | | GAGGGCAAGGCCAGCTCCGCTAAACAGCGCCTGAAGTGTGCTAGCCTGCAGAAATTCGGGGA |
| | | GCGAGCCTTCAAGGCTTGGGCAGTGGCACGGCTGAGTCAGAGATTCCCAAAGGCAGAATTTG |
| | | CCGAGGTCTCAAAACTGGTGACCGACCTGACAAAGGTGCACACCGAATGCTGTCATGGCGACC |
| | | TGCTGGAGTGCGCCGACGATCGAGCTGATCTGGCAAAGTATATTTGTGAGAACCAGGACTCC |
| | | ATCTCTAGTAAGCTGAAAGAATGCTGTGAGAAACCACTGCTGGAAAAGTCTCACTGCATTGC |
| | | CGAAGTGGAGAACGACGAGATGCCAGCTGATCTGCCCTCACTGGCCGCTGACTTCGTCGAAAG |
| | | CAAAGATGTGTGTAAGAATTACGCTGAGGCAAAGGATGTGTTCCTGGGAATGTTTCTGTACG |
| | | AGTATGCCAGGCGCCACCCAGACTACTCCGTGGTCCTGCTGCTGAGGCTGGCTAAAACATATG |
| | | AAACCACACTGGAGAAGTGCTGTGCAGCCGCTGATCCCCATGAATGCTATGCCAAAGTCTTCG |
| | | ACGAGTTTAAGCCCCTGGTGGAGGAACCTCAGAACCTGATCAAACAGAATTGTGAACTGTTT |
| | | GAGCAGCTGGGCGAGTACAAGTTCCAGAACGCCCTGCTGGTGCGCTATACCAAGAAAGTCCCA |
| | | CAGGTGTCCACACCCACTCTGGTGGAGGTGAGCCGGAATCTGGGCAAAGTGGGGAGTAAATG |
| | | CTGTAAGCACCCTGAAGCCAAGAGGATGCCATGCGCTGAGGATTACCTGAGTGTGGTCCTGA |
| | | ATCAGCTGTGTGTCCTGCATGAAAAAACACCTGTCAGCGACCGGGTGACAAAGTGCTGTACT |
| | | GAGTCACTGGTGAACCGACGGCCCTGCTTTAGCGCCCTGGAAGTCGATGAGACTTATGTGCCT |
| | | AAAGAGTTCAACGCTGAGACCTTCACATTTCACGCAGACATTTGTACCCTGAGCGAAAAGGA |
| | | GAGACAGATCAAGAAACAGACAGCCCTGGTCGAACTGGTGAAGCATAAACCCAAGGCCACAA |
| | | AAGAGCAGCTGAAGGCTGTCATGGACGATTTCGCAGCCTTTGTGGAAAAATGCTGTAAGGCA |
| | | GACGATAAGGAGACTTGCTTTGCCGAGGAAGGAAAGAAACTGGTGGCTGCATCCCAGGCAGC |
| | | TCTGGGACTG |
| 38 | hFc/hIL-2 fusion | EPKSCDKTHTCPPCPAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGKGGGSAPTSSSTKKTQLQLE |
| | | HLLLDLQMILNGINNYKNPKLTRMLTFKFYMPKKATELKHLQCLEEELKPLEEVLNLAQSKNF |
| | | HLRPRDLISNINVIVLELKGSETTFMCEYADETATIVEFLNRWITFCQSIISTLT |
| 39 | hIL-2/hFc | APTSSSTKKTQLQLEHLLLDLQMILNGINNYKNPKLTRMLTFKFYMPKKATELKHLQCLEEEL |
| | | KPLEEVLNLAQSKNEHLRPRDLISNINVIVLELKGSETTFMCEYADETATIVEFLNRWITECQSII |
| | | STLTGGGSEPKSCDKTHTCPPCPAPELLGGPSVFLEPPKPKDTLMISRTPEVTCVVVDVSHEDP |
| | | EVKFNWYVDGVEVHNAKTKPREEQYNSTYRWSVLTVLHQDWLNGKEYKCKVSNKALPAPIE |
| | | KTISKAKGQPREPQVYTLPPSRDELTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| 40 | HSA/hIL-2 fusion | DAHKSEVAHRFKDLGEENFKALVLIAFAQYLQQCPFEDHVKLVNEVTEFAKTCVADESAENCD |
| | | KSLHTLFGDKLCTVATLRETYGEMADCCAKQEPERNECFLQHKDDNPNLPRLVRPEVDVMCT |
| | | AFHDNEETFLKKYLYEIARRHPYFYAPELLFFAKRYKAAFTECCQAADKAACLLPKLDELRDE |
| | | GKASSAKQRLKCASLQKFGERAFKAWAVARLSQRFPKAEFAEVSKLVTDLTKVHTECCHGDLL |
| | | ECADDRADLAKYICENQDSISSKLKECCEKPLLEKSHCIAEVENDEMPADLPSLAADEVESKDV |
| | | CKNYAEAKDVFLGMFLYEYARRHPDYSWLLLRLAKTYETTLEKCCAAADPHECYAKVFDEFK |
| | | PLVEEPQNLIKQNCELFEQLGEYKFQNALLVRYTKKVPQVSTPTLVEVSRNLGKVGSKCCKHPE |
| | | AKRMPCAEDYLSWLNQLCVLHEKTPVSDRVTKCCTESLVNRRPCFSALEVDETYVPKEFNAE |
| | | TFTFHADICTLSEKERQIKKQTALVELVKHKPKATKEQLKAVMDDFAAFVEKCCKADDKETCF |
| | | AEEGKKLVAASQAALGLGGGSAPTSSSTKKTQLQLEHLLLDLQMILNGINNYKNPKLTRMLTF |
| | | KEYMPKKATELKHLQCLEEELKPLEEVLNLAQSKNEHLRPRDLISNINVIVLELKGSETTFMCE |
| | | YADETATIVEFLNRWITFCQSIISTLT |
| 41 | hIL-2/HSA fusion | APTSSSTKKTQLQLEHLLLDLQMILNGINNYKNPKLTRMLTFKFYMPKKATELKHLQCLEEEL |
| | | KPLEEVLNLAQSKNEHLRPRDLISNINVIVLELKGSETTFMCEYADETATIVEFLNRWITECQSII |
| | | STLTGGGSDAHKSEVAHRFKDLGEENFKALVLIAFAQYLQQCPFEDHVKLVNEVTEFAKTCVA |
| | | DESAENCDKSLHTLFGDKLCTVATLRETYGEMADCCAKQEPERNECFLQHKDDNPNLPRLVR |
| | | PEVDVMCTAFHDNEETFLKKYLYEIARRHPYFYAPELLFFAKRYKAAFTECCQAADKAACLLP |
| | | KLDELRDEGKASSAKQRLKCASLQKFGERAFKAWAVARLSQRFPKAEFAEVSKLVTDLTKVH |
| | | TECCHGDLLECADDRADLAKYICENQDSISSKLKECCEKPLLEKSHCIAEVENDEMPADLPSLA |
| | | ADEVESKDVCKNYAEAKDVFLGMFLYEYARRHPDYSVVLLLRLAKTYETTLEKCCAAADPHEC |
| | | YAKVFDEFKPLVEEPQNLIKQNCELFEQLGEYKFQNALLVRYTKKVPQVSTPTLVEVSRNLGK |
| | | VGSKCCKHPEAKRMPCAEDYLSVVLNQLCVLHEKTPVSDRVTKCCTESLVNRRPCFSALEVDE |
| | | TYVPKEFNAETFTFHADICTLSEKERQIKKQTALVELVKHKPKATKEQLKAVMDDFAAFVEKC |
| | | CKADDKETCFAEEGKKLVAASQAALGL |

1. A method of prolonging persistence of transferred cells, stimulating the proliferation of transferred cells, or stimulating a T cell-mediated immune response to a target cell population in a cancer subject receiving adoptive cell therapy (ACT), comprising:
administering an extended-pharmacokinetic (PK) interleukin (IL)-2 to a cancer subject receiving ACT, in an amount effective to prolong the persistence of transferred cells in the subject.

2-3. (canceled)

4. A method of treating cancer or promoting tumor regression in a subject, comprising administering to the subject an adoptive cell therapy (ACT), and an extended-pharmacokinetic (PK) interleukin (IL)-2, in an amount effective to treat cancer or promote tumor regression.

5. (canceled)

6. The method of claim 1, further comprising administering a therapeutic antibody or antibody fragment which specifically recognizes a tumor antigen to the subject.

7. (canceled)

8. The method of claim 1, wherein the ACT comprises administration of autologous cells selected from the group consisting of autologous T cells, tumor infiltrating lymphocytes that have been expanded in vitro, CD8+ T cells that have been expanded in vitro in the presence of antigen, CD4+ T cells that have been expanded in vitro in the presence of antigen, and genetically engineered T cells.

9-12. (canceled)

13. The method of claim 8, wherein the genetically engineered T cells have been engineered to express a T cell receptor (TCR) that specifically recognizes a tumor antigen or a chimeric antigen receptor (CAR).

14. (canceled)

15. The method of claim 13, wherein the CAR comprises an antigen binding domain, a costimulatory domain, and a CD3 zeta signaling domain.

16. The method of claim 15, wherein the antigen binding domain is an antibody or antibody fragment that specifically binds to a tumor antigen.

17. The method of claim 16, wherein the antibody fragment is a Fab or an scFv.

18. The method of claim 15, wherein the costimulatory domain comprises the intracellular domain of a costimulatory molecule selected from the group consisting of 4-1BB, CD27, CD28, OX40, CD30, CD40, PD-1, ICOS, lymphocyte function-associated antigen-1 (LFA-1), CD2, CD7, LIGHT, NKG2C, B7-H3, a CD83 ligand, and combinations thereof.

19. (canceled)

20. The method of claim 6, wherein the tumor antigen is an antigen associated with a cancer selected from the group consisting of a hematological tumor, a carcinoma, a blastoma, and a sarcoma.

21. The method of claim 20, wherein the tumor antigen is associated with melanoma or acute myelogenous leukemia.

22. The method of claim 20, wherein the tumor antigen is selected from the group consisting of MART-1, gp100, p53, NY-ESO-1, TRP-1, TRP-2, tyrosinase, CD19, and TRP-1.

23. The method of claim 1, wherein the transferred cells persist for 50% longer in the subject relative to a subject receiving ACT monotherapy.

24. A method of prolonging persistence of transferred cells in a cancer subject receiving adoptive cell therapy (ACT), comprising:
administering an extended-pharmacokinetic (PK) interleukin (IL)-2 to a cancer subject receiving ACT, wherein ACT comprises administration of autologous T cells genetically engineered to express a chimeric antigen receptor (CAR); and
administering a therapeutic antibody to the subject, wherein the therapeutic antibody and the CAR recognize the same tumor antigen;
such that the persistence of transferred cells in the subject is prolonged.

25. The method of claim 1, wherein the extended-PK IL-2 comprises a fusion protein.

26. The method of claim 25, wherein the fusion protein comprises an IL-2 moiety and a moiety selected from the group consisting of an immunoglobulin fragment, human serum albumin, and Fn3.

27. The method of claim 1, wherein the extended-PK IL-2 comprises an IL-2 moiety conjugated to a non-protein polymer.

28. The method of claim 27, wherein the non-protein polymer is polyethylene glycol.

29. The method of claim 26, wherein the fusion protein comprises an IL-2 moiety and an Fc domain.

30. The method of claim 29, wherein the Fc domain is mutated to reduce binding to Fcγ receptors, complement proteins, or both.

31. The method of claim 30, wherein the fusion protein comprises a monomer of one IL-2 moiety linked to an Fc domain as a heterodimer or a dimer of two IL-2 moieties linked to an Fc domain as a heterodimer.

32. (canceled)

33. The method of claim 1, wherein the IL-2 is mutated such that it has higher affinity for the IL-2R alpha receptor compared to unmodified IL-2.
